(19) United States
(12) Patent Application Publication
(10) Pub. No.: US 2025/0176876 A1
(43) Pub. Date: Jun. 5, 2025

(54) ENDOSCOPE SYSTEM AND METHOD FOR OPERATING THE SAME

(71) Applicant: FUJIFILM Corporation, Tokyo (JP)

(72) Inventor: Takahiro OKAMOTO, Kanagawa (JP)

(73) Assignee: FUJIFILM Corporation, Tokyo (JP)

(21) Appl. No.: 19/048,953

(22) Filed: Feb. 9, 2025

Related U.S. Application Data
(63) Continuation of application No. PCT/JP2023/021964, filed on Jun. 13, 2023.

(30) Foreign Application Priority Data
Aug. 12, 2022 (JP) 2022-129056

(51) Int. Cl. A61B 5/1459; A61B 1/00; A61B 5/00; A61B 5/1455

(52) U.S. Cl. CPC A61B 5/1459; A61B 1/00009; A61B 5/14552; A61B 5/743; A61B 5/7485

(57) ABSTRACT

An endoscope system includes an endoscope that generates an image signal, and a processor. The processor is configured to set a plurality of regions of interest in an endoscopic image, store the positions of the regions of interest as region position information, calculate a biological index value based on the image signal in the regions of interest, calculate, for each the regions of interest, a region index value that is a statistical value of the biological index value, generate an index value display table in which a plurality of the region index values are collected, and perform control to display a display image that displays the endoscopic image, the index value display table, and a plurality of pieces of the region position information.

### CROSS-REFERENCE TO RELATED APPLICATIONS

This application is a Continuation of PCT International Application No. PCT/JP2023/021964 filed on 13 Jun. 2023, which claims priority under 35 U.S.C § 119 (a) to Japanese Patent Application No. 2022-129056 filed on 12 Aug. 2022. The above application is hereby expressly incorporated by reference, in its entirety, into the present application.

### BACKGROUND OF THE INVENTION

### 1. Field of the Invention

The disclosure relates to an endoscope system for performing control to display an index value indicating a state of a living body, and a method for operating the endoscope system.

### 2. Description of the Related Art

One approach for supporting surgical treatment is a method of calculating and displaying an index value indicating a state of a living body on the basis of an endoscopic image captured using a rigid endoscope. For example, JP2012-235926A (corresponding to US2012/0289801A1) discloses a medical apparatus system including image acquiring means for acquiring a subject image including at least two kinds of spectral information relating to wavelengths of light at predetermined time intervals, lock-on setting means for setting a lock-on area that follows the movement of a region of interest of a subject in the region of interest on the subject image, monitoring image generating means for generating a monitoring image to be used for monitoring a change in oxygen saturation over time in the lock-on area on the basis of an image of a lock-on area portion of the subject image, and display means for displaying the monitoring image. The configuration described above ensures the monitoring of a change in oxygen saturation of the subject over time even if the imaging area of the subject changes due to the movement or the like of a tip portion of a laparoscope. In JP2012-235926A, oxygen saturation is calculated as an index value indicating a state of a living body.

### SUMMARY OF THE INVENTION

In the support of surgical treatment using an index value indicating a state of a living body based on an endoscopic image, the monitoring of the index value indicating the state of the living body only for one local region included in the endoscopic image may be insufficient in a situation in which a spatial change in the index value indicating the state of the living body is desired to be known, particularly, in a situation in which the range of excision of a lesion over a wide range is desired to be determined. Accordingly, there is a demand for a technique that enables visual recognition of a spatial change in an index value indicating a state of a living body.

An object of the disclosure is to provide an endoscope system that allows a user to visually recognize a spatial change in an index value indicating a state of a living body, and a method for operating the endoscope system.

An endoscope system according to an exemplary embodiment of the invention includes an endoscope and a processor. The endoscope captures an image of a photographic subject to generate an image signal. The processor is configured to acquire the image signal; generate an endoscopic image based on the image signal; set a plurality of regions of interest at different positions in the endoscopic image; store each of the positions of the plurality of regions of interest in the endoscopic image as region position information; calculate a biological index value indicating a state of the photographic subject, based on the image signal in the regions of interest; calculate, for each of the regions of interest, a region index value that is a statistical value of the biological index value, based on the biological index value in each of the regions of interest; generate an index value display table that collectively displays a plurality of the region index values; generate a display image that displays the endoscopic image, the index value display table, and a plurality of pieces of the region position information; and perform control to display the display image.

The biological index value may be an oxygen saturation and/or a hemoglobin index.

The index value display table may display the plurality of the region index values in a graph format.

The processor may be configured to associate the region position information and the region index value with each other to store the region index value as a specific region index value; and hold the specific region index value and display the specific region index value in the index value display table.

The processor may be configured to calculate the biological index value based on the image signal that is latest in the regions of interest; calculate, for each of the regions of interest, the region index value based on the biological index value that is latest; and update the region index value displayed in the index value display table.

The processor may be configured to associate the region position information and each of the regions of interest with each other to store the position of the region of interest in the endoscopic image as a lock-on area; and calculate the biological index value based on the image signal in the lock-on area.

The processor may be configured to associate the region index value calculated based on the image signal in the lock-on area with the lock-on area to store the region index value as a specific lock-on area index value; and perform control to display the specific lock-on area index value on the display image.

When the lock-on area may be at a position out of a field of view, the position out of the field of view being a position not included in the endoscopic image, the processor is configured to set, as an out-of-field-of-view lock-on area index value, the specific lock-on area index value stored immediately before the lock-on area is located at the position out of the field of view, and generate the index value display table that displays the out-of-field-of-view lock-on area index value.

The processor may be configured to set at least one lock-on area in the endoscopic image as an additional region of interest, each of the at least one lock-on area being the lock-on area; calculate the biological index value based on the image signal in the additional region of interest; calculate an additional region index value as the region index value, the additional region index value being a statistical value of the biological index value in the additional region of interest; generate an extension index value display table that collectively displays the additional region index value and the out-of-field-of-view lock-on area index value; and perform control to display the extension index value display table on the display image.

The processor may be configured to perform control to display a plurality of pieces of the region position information in a superimposed manner on the endoscopic image; and perform control to display an index value link line on the display image, the index value link line connecting the region position information displayed in a superimposed manner on the endoscopic image and the additional region index value other than the out-of-field-of-view lock-on area index value, the additional region index value other than the out-of-field-of-view lock-on area index value being displayed in the extension index value display table and corresponding to the region position information.

The processor may be configured to perform control to change a display size of the extension index value display table displayed on the display image.

The processor may be configured to perform control to display a plurality of pieces of the region position information in a superimposed manner on the endoscopic image; and perform control to display an index value link line on the display image, the index value link line connecting the region position information displayed in a superimposed manner on the endoscopic image and the region index value displayed in the index value display table and corresponding to the region position information.

The processor may be configured to set at least one lock-on area in the endoscopic image as an additional region of interest, each of the at least one lock-on area being the lock-on area; calculate the biological index value based on the image signal in the additional region of interest; calculate an additional region index value as the region index value, the additional region index value being a statistical value of the biological index value in the additional region of interest; generate the index value display table that displays the additional region index value; and perform control to display, on the display image, the index value display table that displays the additional region index value.

The endoscope system may further include a region-of-interest setting switch, and the processor is configured to set the plurality of regions of interest in accordance with a pressing of the region-of-interest setting switch; and calculate the region index value in the set regions of interest in accordance with a further pressing of the region-of-interest setting switch.

A method for operating an endoscope system according to an exemplary embodiment of the invention includes the steps of acquiring an image signal generated by an endoscope capturing an image of a photographic subject; generating an endoscopic image based on the image signal; setting a plurality of regions of interest at different positions in the endoscopic image; storing each of the positions of the plurality of regions of interest in the endoscopic image as region position information; calculating a biological index value indicating a state of the photographic subject, based on the image signal in the regions of interest; calculating, for each of the regions of interest, a region index value that is a statistical value of the biological index value, based on the biological index value in each of the regions of interest; generating an index value display table that collectively displays a plurality of the region index values; generating a display image that displays the endoscopic image, the index value display table, and a plurality of pieces of the region position information; and performing control to display the display image.

The exemplary embodiments of the invention allow a user to visually recognize a spatial change in an index value indicating a state of a living body.

### BRIEF DESCRIPTION OF THE DRAWINGS

FIG. 1 is a schematic diagram of an endoscope system;

FIG. 2 is a schematic diagram of an endoscope system according to a first embodiment;

FIG. 3 is a schematic diagram of an endoscope system including an endoscope that is a laparoscope;

FIG. 4 is a block diagram illustrating functions of the endoscope system according to the first embodiment;

FIG. 5 is a block diagram illustrating functions of a light source unit;

FIG. 6 is an explanatory diagram illustrating an example presentation on a display in a normal mode;

FIG. 7 is an explanatory diagram illustrating an example presentation on the display in an oxygen saturation mode;

FIG. 8 is an image diagram illustrating an example of a notification image for prompting switching to a correction mode;

FIG. 9 is an explanatory diagram illustrating an example presentation on the display in the correction mode;

FIG. 10 is a graph illustrating the spectrum of white light;

FIG. 11 is a graph illustrating the spectrum of first illumination light;

FIG. 12 is a graph illustrating the spectrum of second illumination light;

FIG. 13 is a graph illustrating the spectrum of third illumination light;

FIG. 14 is an explanatory diagram illustrating an example of a normal-mode light emission pattern;

FIG. 15 is an explanatory diagram illustrating an example of an oxygen-saturation-mode light emission pattern;

FIG. 16 is an explanatory diagram illustrating an example of a correction-mode light emission pattern;

FIG. 17 is a graph illustrating transmission bands of color filters of an imaging sensor according to the first embodiment;

FIG. 18 is a table illustrating illumination light emitted in the normal mode according to the first embodiment and acquired image signals;

FIG. 19 is a table illustrating illumination light emitted in the oxygen saturation mode according to the first embodiment and acquired image signals;

FIG. 20 is a table illustrating illumination light emitted in the correction mode according to the first embodiment and acquired image signals;

FIG. 21 is a graph illustrating hemoglobin reflection spectra in a case where a specific pigment is not included in a biological tissue;

FIG. 22A is a graph illustrating reflection spectra of deoxyhemoglobin in the presence of a yellow pigment;

FIG. 22B is a graph illustrating an absorption spectrum of the yellow pigment;

FIG. 23 is a table illustrating the oxygen saturation dependence, blood concentration dependence, and brightness dependence of a B1 image signal, a G2 image signal, and an R2 image signal;

FIG. 24 is a graph illustrating an oxygen saturation calculation table;

FIG. 25 is a table illustrating the oxygen saturation dependence, blood concentration dependence, and brightness dependence of an X-component value (signal ratio ln(R2/G2)) and a Y-component value (signal ratio ln(B1/G2));

FIG. 26 is a table illustrating the oxygen saturation dependence, blood concentration dependence, brightness dependence, and yellow pigment dependence of the B1 image signal, the G2 image signal, and the R2 image signal;

FIG. 27 is a graph illustrating that the apparent oxygen saturation is calculated to be high due to the presence of the yellow pigment;

FIG. 28 is a table illustrating the oxygen saturation dependence, blood concentration dependence, brightness dependence, and yellow pigment dependence of the B1 image signal, the G2 image signal, the R2 image signal, a B3 image signal, a G3 image signal, and a B2 image signal;

FIG. 29A is a graph illustrating a corrected oxygen saturation calculation table in a three-dimensional coordinate system;

FIG. 29B is a graph illustrating a corrected oxygen saturation calculation table obtained by representing that of FIG. 29A in a two-dimensional coordinate system;

FIG. 30 is a table illustrating the oxygen saturation dependence, blood concentration dependence, brightness dependence, and yellow pigment dependence of the X-component value (signal ratio ln(R2/G2)), the Y-component value (signal ratio ln(B1/G2)), and a Z-component value (signal ratio ln(B3/G3));

FIG. 31 is a block diagram illustrating functions of an extension processor device;

FIG. 32 is an explanatory diagram illustrating a method for calculating an oxygen saturation;

FIG. 33A is a graph illustrating a corrected oxygen saturation calculation table in a two-dimensional coordinate system;

FIG. 33B is a graph illustrating an example of an oxygen saturation calculation table of an area AR2;

FIG. 34 is an explanatory diagram illustrating a method for calculating a corrected oxygen saturation;

FIG. 35 is an image diagram illustrating an example of a correction image;

FIG. 36 is a graph illustrating a first reliability calculation table;

FIG. 37 is a graph illustrating a second reliability calculation table;

FIG. 38 is a graph illustrating a third reliability calculation table;

FIG. 39 is an image diagram illustrating an example of a correction image whose saturation is to be changed according to the reliability;

FIG. 40 is an image diagram illustrating an example of a correction image in which a specific region is surrounded by a frame according to the reliability;

FIG. 41 is an image diagram illustrating an example of a correction image in which it is indicated that correction processing can be appropriately performed;

FIG. 42 is an image diagram illustrating an example of a correction image in which a warning is displayed;

FIG. 43A is an image diagram illustrating an example of an oxygen saturation image;

FIG. 43B is an image diagram illustrating an example of a region-of-interest image in specific example (1);

FIG. 44 is an explanatory diagram illustrating an example presentation on the display in a case where the region-of-interest image in the specific example (1) is displayed;

FIG. 45 is a graph illustrating a combination index calculation table;

FIG. 46 is an image diagram illustrating an example of a biological index value selection screen;

FIG. 47 is an explanatory diagram illustrating an example of an index value display table in a graph format;

FIG. 48 is an explanatory diagram illustrating an example of an index value display table in a table format;

FIG. 49 is an image diagram illustrating an example of a display image in the specific example (1);

FIG. 50 is an explanatory diagram illustrating an example presentation on the display in a case where the display image in the specific example (1) is displayed;

FIG. 51 is an explanatory diagram illustrating an example presentation on the display in a case where a display image in specific example (2) is displayed;

FIG. 52 is an explanatory diagram illustrating an example presentation on the display in a case where a region-of-interest image in specific example (3) is displayed;

FIG. 53 is an explanatory diagram illustrating an example presentation on the display in a case where a display image in specific example (3) is displayed;

FIG. 54 is an image diagram illustrating an example of a display image in a case where region index values related to a plurality of types of biological index values are calculated;

FIG. 55 is a flowchart illustrating a process flow for displaying a display image according to the first embodiment;

FIG. 56 is an image diagram illustrating an example of a region-of-interest image for displaying a display region of interest;

FIG. 57 is an image diagram illustrating an example of a display image in a case where display-region position information is displayed;

FIG. 58 is a block diagram illustrating functions of an extension processor device provided with a region index value storage unit;

FIG. 59 is an image diagram illustrating an example of a display image in a case where a region index value is updated;

FIG. 60 is an explanatory diagram illustrating an example of region index values calculated in chronological order in a case where illumination light is emitted in the oxygen-saturation-mode light emission pattern;

FIG. 61A is an image diagram illustrating an example of a region-of-interest image;

FIG. 61B is an image diagram illustrating an example of a region-of-interest image in which regions of interest in FIG. 61A follow the movement of the endoscope;

FIG. 62A is an image diagram illustrating an example of a region-of-interest image for displaying a display region of interest;

FIG. 62B is an image diagram illustrating an example of a region-of-interest image for displaying a display region of interest when regions of interest in FIG. 62A follow the movement of the endoscope;

FIG. 63A is an image diagram illustrating an example of a display image at a time point before an out-of-field-of-view lock-on area index value is displayed;

FIG. 63B is an image diagram illustrating an example of a display image in a case where the out-of-field-of-view lock-on area index value at a time point after that of FIG. 63A is displayed;

FIG. 64 is an image diagram illustrating an example of a display image in a case where an additional region of interest is set when the out-of-field-of-view lock-on area index value is displayed;

FIG. 65 is a block diagram illustrating functions of an index value link line generation unit;

FIG. 66 is an image diagram illustrating an example of a display image in a case where the out-of-field-of-view lock-on area index value and index value link lines are displayed;

FIG. 67 is an image diagram illustrating an example of a display image in a case where the display size of the index value display table is changed when the out-of-field-of-view lock-on area index value is displayed;

FIG. 68 is an image diagram illustrating an example of a display image in a case where the index value link lines are displayed when the out-of-field-of-view lock-on area index value and the additional region of interest are not displayed;

FIG. 69 is an image diagram illustrating an example of a display image after the endoscope is moved when the out-of-field-of-view lock-on area index value and the additional region of interest are not displayed;

FIG. 70 is an image diagram illustrating an example of a display image in a case where the endoscope is moved and the display size of the index value display table is changed when the out-of-field-of-view lock-on area index value and the additional region of interest are not displayed;

FIG. 71 is an image diagram illustrating an example of a display image in a case where the index value link lines and display-region position information are displayed when the out-of-field-of-view lock-on area index value and the additional region of interest are not displayed;

FIG. 72 is an image diagram illustrating an example of a display image in a case where the index value link lines and display-region position information are displayed after the endoscope is moved when the out-of-field-of-view lock-on area index value and the additional region of interest are not displayed;

FIG. 73 is an image diagram illustrating an example of a display image in which display-region position information is displayed in a case where the endoscope is moved and the display size of the index value display table is changed when the out-of-field-of-view lock-on area index value and the additional region of interest are not displayed;

FIG. 74 is an image diagram illustrating an example of a display image in a case where the out-of-field-of-view lock-on area index value is not displayed;

FIG. 75 is an image diagram illustrating an example of a display image in which additional regions of interest are set when the out-of-field-of-view lock-on area index value is not displayed;

FIG. 76 is an image diagram illustrating an example of a display image in which additional region index values are displayed when no out-of-field-of-view lock-on area index value is displayed;

FIG. 77 is an image diagram illustrating an example of a display image in which display-region position information is displayed when the out-of-field-of-view lock-on area index value is not displayed;

FIG. 78 is an image diagram illustrating an example of a display image in which display-region position information is displayed in a case where the additional regions of interest are set when the out-of-field-of-view lock-on area index value is not displayed;

FIG. 79 is an image diagram illustrating an example of a display image in which display-region position information is displayed in a case where the additional region index values are displayed when the out-of-field-of-view lock-on area index value is not displayed;

FIG. 80 is a block diagram illustrating functions of an endoscope system according to a second embodiment;

FIG. 81 is a plan view of a rotary filter;

FIG. 82 is an explanatory diagram illustrating a difference value AZ to be used for calculation value correction processing;

FIG. 83 is a schematic diagram of an endoscope system according to a third embodiment;

FIG. 84 is a graph illustrating the spectrum of mixed light including first blue light, second blue light, green light, and red light;

FIG. 85 is an explanatory diagram illustrating functions of a camera head according to the third embodiment;

FIG. 86 is a graph illustrating the spectrum of light incident on an imaging sensor 511;

FIG. 87 is a graph illustrating the spectrum of light incident on an imaging sensor 512;

FIG. 88 is a graph illustrating the spectrum of light incident on an imaging sensor 513;

FIG. 89 is a graph illustrating the spectrum of light incident on an imaging sensor 514;

FIG. 90 is a graph illustrating reflection spectra of deoxyhemoglobin in the presence of a yellow pigment, in which a wavelength range Rk is displayed;

FIG. 91 is a table illustrating the oxygen saturation dependence, blood concentration dependence, brightness dependence, and yellow pigment dependence of the G2 image signal, the G3 image signal, the R2 image signal, and an Rk image signal;

FIG. 92 is an explanatory diagram illustrating functions of a camera head according to a fourth embodiment;

FIG. 93A is a graph illustrating the spectrum of fourth illumination light;

FIG. 93B is a graph illustrating the relationship between the reflectance and transmittance of light incident on a dichroic mirror according to the fourth embodiment and the wavelength of the light;

FIG. 93C is a graph illustrating the relationship between the sensitivity of an imaging sensor 611 and the wavelength of light;

FIG. 94A is a graph illustrating the spectrum of the fourth illumination light;

FIG. 94B is a graph illustrating the relationship between the reflectance and transmittance of light incident on a dichroic mirror according to the fourth embodiment and the wavelength of the light;

FIG. 94C is a graph illustrating the relationship between the sensitivity of an imaging sensor 612 and the wavelength of light;

FIG. 95 is an explanatory diagram illustrating a light emission pattern in the oxygen saturation mode according to the fourth embodiment;

FIG. 96 is an explanatory diagram illustrating a light emission pattern in the correction mode according to the fourth embodiment;

FIG. 97A is a graph illustrating the spectrum of third illumination light;

FIG. 97B is a graph illustrating the relationship between the reflectance and transmittance of light incident on a dichroic mirror according to the fourth embodiment and the wavelength of the light;

FIG. 97C is a graph illustrating the relationship between the sensitivity of the imaging sensor 611 and the wavelength of light;

FIG. 98A is a graph illustrating the spectrum of the third illumination light;

FIG. 98B is a graph illustrating the relationship between the reflectance and transmittance of light incident on a dichroic mirror according to the fourth embodiment and the wavelength of the light;

FIG. 98C is a graph illustrating the relationship between the sensitivity of the imaging sensor 612 and the wavelength of light;

FIG. 99 is a block diagram illustrating functions of an endoscope system according to the fourth embodiment;

FIG. 100A is an image diagram illustrating a correction region;

FIG. 100B is an enlarged view of the correction region illustrated in FIG. 100A;

FIG. 101 is a block diagram illustrating functions of a reliability calculation unit, a correction determination unit, and an extension display control unit according to the fourth embodiment;

FIG. 102 is a graph illustrating a first reliability calculation table, with the horizontal axis representing the pixel value of the G2 image signal;

FIG. 103 is a graph illustrating a third reliability calculation table, with the vertical axis representing the signal ratio ln(B1/G2);

FIG. 104 is an explanatory diagram illustrating the relationship among a light emission pattern in the correction mode according to the fourth embodiment, an endoscopic image to be generated, and image sets;

FIG. 105 is an explanatory diagram illustrating corresponding correction regions of a white-light-equivalent image, a first blue light image, and a third illumination light image;

FIG. 106 is an explanatory diagram illustrating a method for calculating a correlation coefficient; and

FIG. 107 is an image diagram illustrating an example presentation on the display for displaying a warning according to the fourth embodiment.

### DESCRIPTION OF THE PREFERRED EMBODIMENTS

### First Embodiment

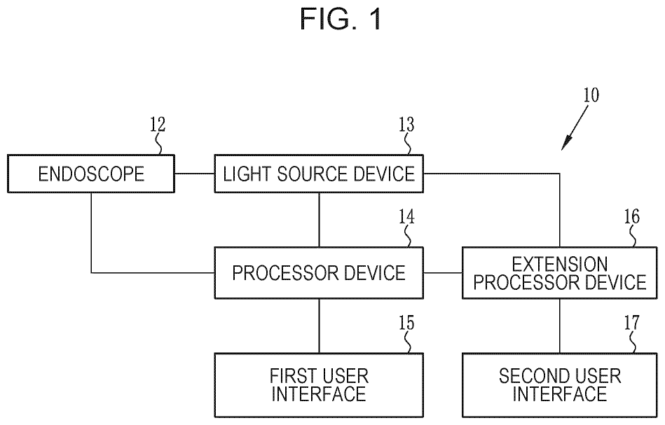

As illustrated in FIG. 1, an endoscope system 10 includes an endoscope 12, a light source device 13, a processor device 14, a first user interface 15, an extension processor device 16, and a second user interface 17. The endoscope 12 is optically or electrically connected to the light source device 13 and is electrically connected to the processor device 14. The first user interface 15 is electrically connected to the processor device 14. The extension processor device 16 is electrically connected to the light source device 13, the processor device 14, and the second user interface 17. The connections described above are not limited to wired connections, and may be wireless connections. The connections may be established via a network.

In the first embodiment, the endoscope system 10 is suitable for use as a rigid endoscope, particularly, a laparoscope. In the rigid endoscope, the endoscope 12 is inserted into a body cavity of a subject to perform surgical treatment, and an image of an organ in the body cavity is captured from the serosa side. The endoscope 12 may be a flexible endoscope that is inserted from the nose, mouth, or anus of the subject. In this specification, the subject means a target into which the endoscope 12 is to be inserted. The photographic subject means an observation target included in the angle of view of the endoscope 12 and appearing in the endoscopic image.

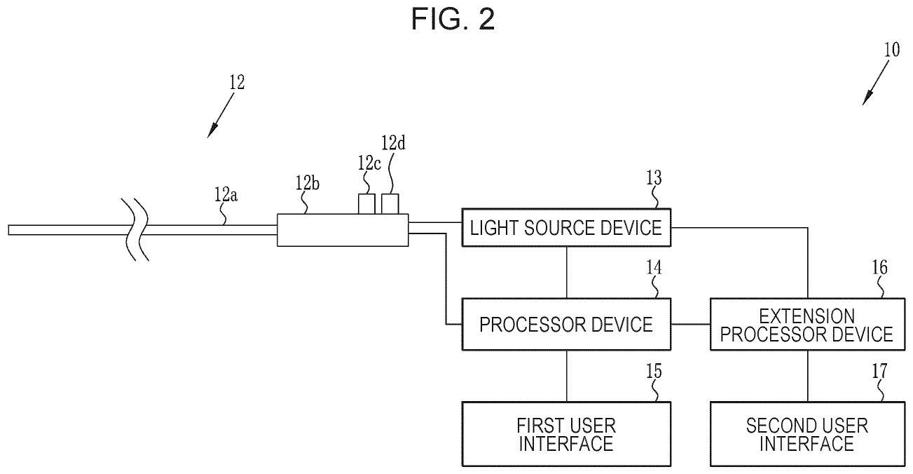

When the endoscope 12 is a laparoscope, as illustrated in FIG. 2, the endoscope 12 includes an insertion section 12a to be inserted into the abdominal cavity of the subject, and an operation section 12b provided in a proximal end portion of the insertion section 12a. A portion near a distal end (hereinafter referred to as a tip portion) of the insertion section 12a has incorporated therein an optical system and an imaging sensor. The optical system includes an illumination optical system described below for irradiating the photographic subject with illumination light, and an imaging optical system described below for capturing an image of the photographic subject. The imaging sensor forms an image of reflected light from the observation target, which results from light incident through the imaging optical system, on an image forming surface to generate an image signal. The generated image signal is output to the processor device 14.

The operation section 12b is provided with a mode switching switch 12c and a region-of-interest setting switch 12d. The mode switching switch 12c is used for a mode switching operation described below. The region-of-interest setting switch 12d is used to input a region-of-interest setting instruction described below and to input an instruction to calculate a biological index value in a region of interest. The region-of-interest setting switch 12d may be operated to perform mode switching without using the mode switching switch 12c, which will be described in detail below.

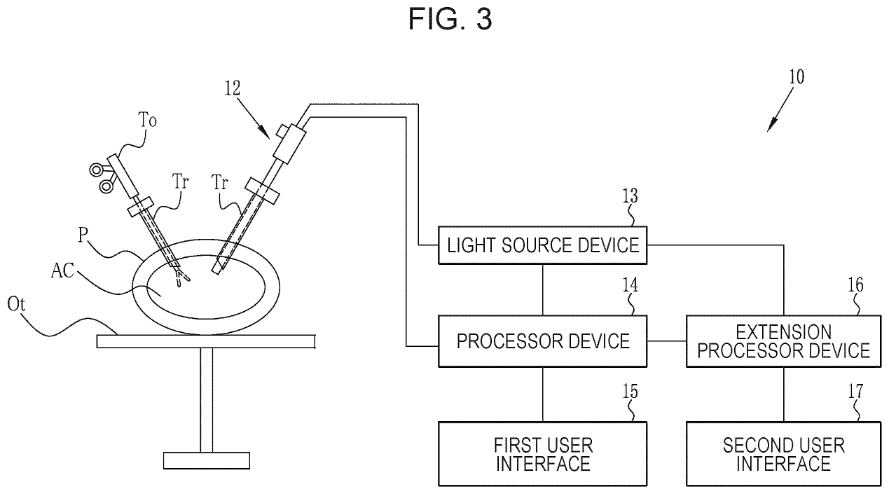

In surgical treatment using a laparoscope, as illustrated in FIG. 3, the endoscope 12 is inserted into an abdominal cavity AC of a subject P who is in a supine position (lying on their back with their face up) on an operating table Ot through a trocar Tr. The abdominal cavity AC of the subject P is inflated with carbon dioxide gas, which is insufflated into the abdominal cavity AC by a pneumoperitoneum apparatus, to secure an observation field and an operation field. Further, a treatment tool To, such as a grasping forceps for expanding the observation field and the operation field or an electric scalpel for removing a part of an organ having a lesion portion, is inserted through a trocar Tr different from the trocar Tr through which the endoscope 12 is inserted.

The light source device 13 generates illumination light. The processor device 14 performs system control of the endoscope system 10 and further performs image processing on image signals transmitted from the endoscope 12 to generate an endoscopic image. In this specification, the “endoscopic image” includes a white-light image, a white-light-equivalent image, an oxygen saturation image, a region-of-interest image, a display image, a correction image, a notification image, a third illumination light image, and a first blue light image.

The first user interface 15 and the second user interface 17 are input devices such as a keyboard, a mouse, a microphone, a foot switch, a touch pad, a tablet, and a touch pen that receive an input operation from a user and transmit an input signal to the processor device 14 or the extension processor device 16. The first user interface 15 and the second user interface 17 are also output devices such as a display, a head-mounted display, and a speaker that receive an output signal from the processor device 14 or the extension processor device 16 and output an endoscopic image, a sound, and the like. Hereinafter, the first user interface 15 and the second user interface 17 are collectively referred to as user interfaces, and the first user interface 15 or the second user interface 17 is referred to as a user interface, unless otherwise specified.

The mode switching switch 12c and the region-of-interest setting switch 12d of the endoscope 12 may be provided in the user interface instead of the endoscope 12.

The endoscope system 10 has three modes, namely, a normal mode, an oxygen saturation mode, and a correction mode. The three modes are switched by the operation of the mode switching switch 12c or the operation of the region-of-interest setting switch 12d by the user.

In the normal mode, a white-light image with a natural color tone generated by imaging the photographic subject using white light as illumination light is displayed on a display serving as the user interface. In the oxygen saturation mode, the oxygen saturation of the photographic subject is calculated, and an oxygen saturation image that is an image of the calculated oxygen saturation is displayed on the display. In the oxygen saturation mode, furthermore, a white-light-equivalent image having fewer short-wavelength components than the white-light image is displayed on the display. In the correction mode, correction processing related to the calculation of the oxygen saturation is performed in consideration of the influence of a specific pigment described below.

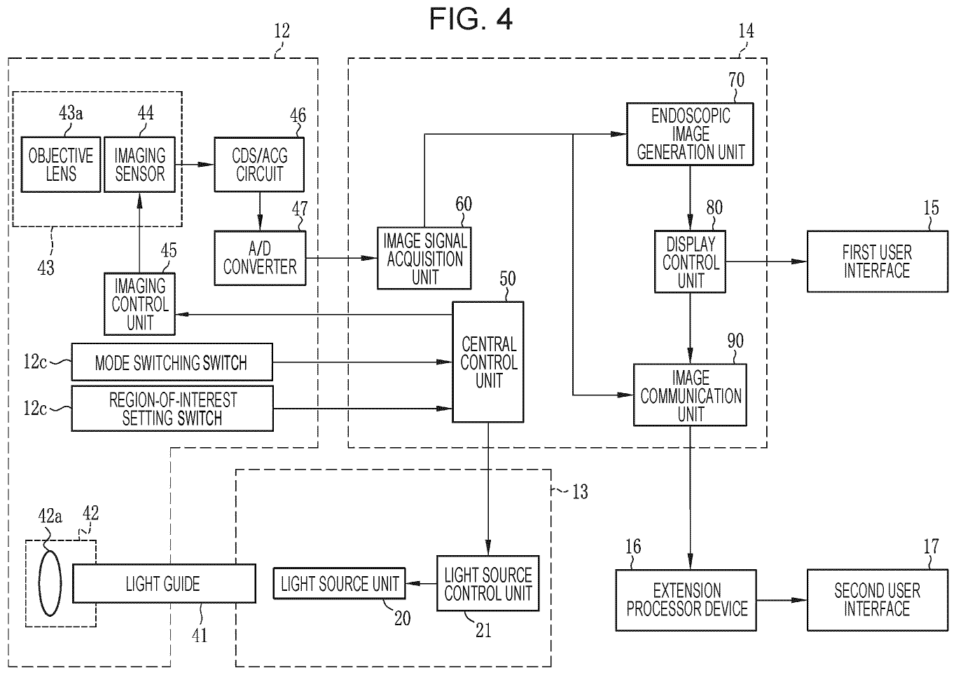

As illustrated in FIG. 4, the light source device 13 has a light source unit 20 and a light source control unit 21 that controls the light source unit 20. The light source unit 20 is constituted by, for example, semiconductor light sources such as light emitting diodes (LEDs) of a plurality of colors, a laser light source, a combination of a laser diode and a fluorescent body, a xenon lamp, a halogen light source, or the like. The light source unit 20 has, for example, a plurality of light sources and turns on or off each of the light sources. When each light source is turned on, the amount of light to be emitted from the light source is controlled by the light source control unit 21. As a result, the light source unit 20 emits illumination light for illuminating the observation target.

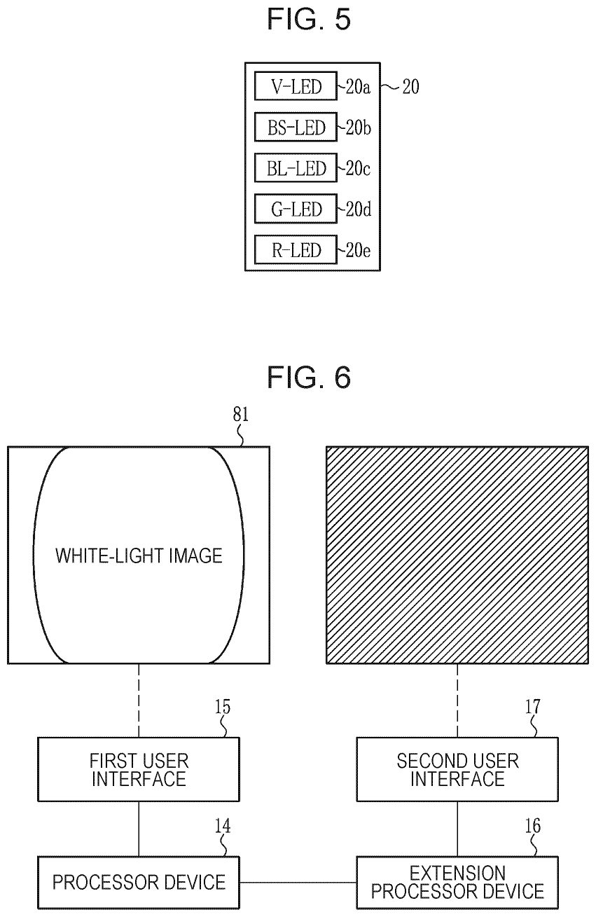

In the first embodiment, as illustrated in FIG. 5, the light source unit 20 has, for example, LEDs of five colors, namely, a violet light emitting diode (V-LED) 20a, a blue short-wavelength light emitting diode (BS-LED) 20b, a blue long-wavelength light emitting diode (BL-LED) 20c, a green light emitting diode (G-LED) 20d, and a red light emitting diode (R-LED) 20e. The combination of the LEDs of the respective colors is not limited to this.

The V-LED 20a emits violet light V having a center wavelength of 410 nm±10 nm. The BS-LED 20b emits second blue light BS having a center wavelength of 450 nm±10 nm. The BL-LED 20c emits first blue light BL having a center wavelength of 470 nm±10 nm. The G-LED 20d emits green light G in the green band. The green light G may have a center wavelength of 540 nm. The R-LED 20e emits red light R in the red band. The red light R may have a center wavelength of 620 nm. The center wavelength and the peak wavelength of each of the LEDs 20a to 20e may be the same or different.

The light source control unit 21 independently inputs a control signal to the LEDs 20a to 20e to independently control the turn-on and turn-off of the LEDs 20a to 20e, the amounts of light to be emitted when the LEDs 20a to 20e are turned on, and the like. The turn-on or turn-off control by the light source control unit 21 is different depending on each mode, and details thereof will be described below.

The illumination light emitted from the light source unit 20 is incident on a light guide 41 through an optical path coupling unit (not illustrated) constituted by a mirror, a lens, and the like. The light guide 41 may be incorporated in the endoscope 12 and a universal cord (a cord that connects the endoscope 12, the light source device 13, and the processor device 14). The light guide 41 propagates the light from the optical path coupling unit to the tip portion of the endoscope 12.

An illumination optical system 42 and an imaging optical system 43 are provided in the tip portion of the endoscope 12. The illumination optical system 42 has an illumination lens 42a, and the illumination light propagated by the light guide 41 is applied to the photographic subject through the illumination lens 42a. When the endoscope 12 is a flexible endoscope and the light source unit 20 is incorporated in the tip portion of the endoscope 12, the illumination light is emitted through the illumination lens 42a of the illumination optical system 42 without the intervention of the light guide 41.

The imaging optical system 43 has an objective lens 43a and an imaging sensor 44. Reflected light from the photographic subject irradiated with the illumination light is incident on the imaging sensor 44 through the objective lens 43a. As a result, an image of the photographic subject is formed on the imaging sensor 44.

The imaging sensor 44 is a color imaging sensor or a monochrome imaging sensor that images the reflected light from the photographic subject. When the imaging sensor 44 is a color imaging sensor, each pixel of the imaging sensor 44 is provided with any one of a B pixel (blue pixel) having a B (blue) color filter, a G pixel (green pixel) having a G (green) color filter, and an R pixel (red pixel) having an R (red) color filter. The wavelength ranges and transmittances of light transmitted through the B color filter, the G color filter, and the R color filter will be described below. In the first embodiment, the imaging sensor 44 may be a color imaging sensor of a Bayer array of B pixels, G pixels, and R pixels, the numbers of which are in the ratio of 1:2:1.

As the imaging sensor 44, a charge coupled device (CCD) imaging sensor, a complementary metal-oxide semiconductor (CMOS) imaging sensor, or the like is applicable. A complementary color imaging sensor including complementary color filters of C (cyan), M (magenta), Y (yellow), and G (green) may be used instead of a color imaging sensor including blue pixels, green pixels, and red pixels. When the complementary color imaging sensor is used, image signals of four CMYG colors are output. In this case, image signals of the four CMYG colors are converted into image signals of three RGB colors by complementary-color-to-primary-color conversion. As a result, image signals of the respective RGB colors similar to those of a color imaging sensor including blue pixels, green pixels, and red pixels, which will be described below, can be obtained. Details of the control of turning on and off the illumination light in each mode and the image signals output from the imaging sensor 44 in each mode will be described below.

Driving of the imaging sensor 44 is controlled by an imaging control unit 45. The control of the imaging sensor 44 by the imaging control unit 45 in each mode will be described below. A correlated double sampling/automatic gain control (CDS/AGC) circuit 46 performs correlated double sampling (CDS) and automatic gain control (AGC) on an analog image signal obtained from the imaging sensor 44. The image signal having passed through the CDS/AGC circuit 46 is converted into a digital image signal by an analog/digital (A/D) converter 47. The image signal after the A/D conversion is input to the processor device 14.

The processor device 14 has a central control unit 50, an image signal acquisition unit 60, an endoscopic image generation unit 70, a display control unit 80, and an image communication unit 90. In the processor device 14, programs related to various types of processing are incorporated in a program memory (not illustrated). The central control unit 50, which is constituted by a processor, executes a program in the program memory to implement the functions of the image signal acquisition unit 60, the endoscopic image generation unit 70, the display control unit 80, and the image communication unit 90.

The image signal acquisition unit 60 acquires an A/D converted image signal from the endoscope 12, and transmits the image signal to the endoscopic image generation unit 70 and/or the image communication unit 90.

The endoscopic image generation unit 70 generates an endoscopic image based on image signals. Specifically, the endoscopic image generation unit 70 performs image processing, which is color conversion processing such as demosaicing, 3×3 matrix processing, gradation transformation processing, or three-dimensional look up table (LUT) processing, and/or structure enhancement processing such as color enhancement processing or spatial frequency enhancement processing, on the image signals of the respective colors to generate an endoscopic image. The demosaicing is processing to generate a signal of a missing color for each pixel. Through the demosaicing, all the pixels have signals of the respective RGB colors. The demosaicing is also performed in the extension processor device 16 described below.

The endoscopic image generation unit 70 performs image processing according to a mode to generate an endoscopic image. In the normal mode, the endoscopic image generation unit 70 performs image processing for the normal mode to generate a white-light image. In the oxygen saturation mode, the endoscopic image generation unit 70 generates a white-light-equivalent image.

In the oxygen saturation mode, the image signal acquisition unit 60 transmits the image signals to the extension processor device 16 via the image communication unit 90. Also in the correction mode, as in the oxygen saturation mode, the endoscopic image generation unit 70 generates a white-light-equivalent image, and the image signals of the respective colors are transmitted to the extension processor device 16 via the image communication unit 90.

The display control unit 80 and an extension display control unit 200 of the extension processor device 16 perform control related to output to the user interface. The display control unit 80 performs display control to display the endoscopic image generated by the endoscopic image generation unit 70 on the display serving as the user interface. The display control unit 80 may perform display control to display an endoscopic image generated by the extension processor device 16 on the display serving as the user interface.

The extension processor device 16 receives the image signals from the processor device 14 and performs various types of image processing. In the oxygen saturation mode, the extension processor device 16 calculates the oxygen saturation and generates an oxygen saturation image that is an image of the calculated oxygen saturation. In the extension processor device 16, the extension display control unit 200 described below performs display control to display an endoscopic image generated by the extension processor device 16 on the display serving as the user interface. In the correction mode, the extension processor device 16 performs correction processing related to the calculation of the oxygen saturation. Details of the processing performed by the extension processor device 16 on the image signals in the oxygen saturation mode and the correction mode will be described below. In the oxygen saturation mode and the correction mode, the extension processor device 16 performs demosaicing on the image signals received from the processor device 14, and then performs calculation of reliability, correction processing, calculation of a biological index value including the oxygen saturation, generation of an oxygen saturation image, generation of a display image, and the like, which will be described below.

In the normal mode, as illustrated in FIG. 6, the display control unit 80 of the processor device 14 displays a white-light image 81 on a display serving as the first user interface 15. In contrast, nothing is displayed on a display serving as the second user interface 17. In the example illustrated in FIG. 6, no display on the display serving as the second user interface 17 is indicated by hatching.

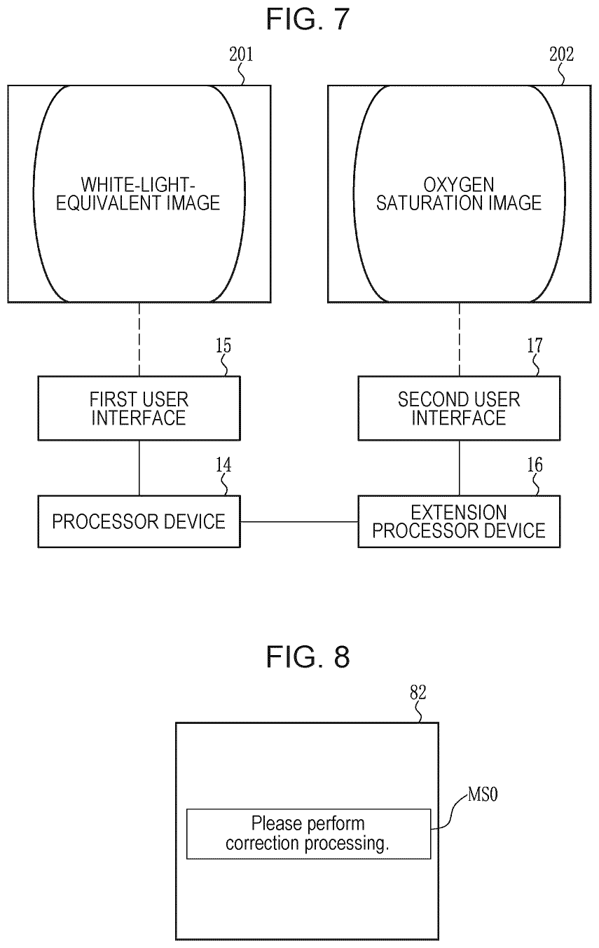

In the oxygen saturation mode, as illustrated in FIG. 7, the display control unit 80 of the processor device 14 displays a white-light-equivalent image 201 on the display serving as the first user interface 15. The extension display control unit 200 of the extension processor device 16 displays an oxygen saturation image 202 on the display serving as the second user interface 17. The extension display control unit 200 of the extension processor device 16 displays a display image or a region-of-interest image described below on the display serving as the second user interface 17. The display control by the extension display control unit 200 will be described in detail below.

When the mode is switched to the oxygen saturation mode, the display control unit 80 of the processor device 14 may display a notification image 82 as illustrated in FIG. 8 for displaying a message MS0, such as “Please perform correction processing”, on the display serving as the user interface and prompt the user to switch the mode to the correction mode. In the oxygen saturation mode, it is preferable to display the oxygen saturation image on the display after the correction processing is performed in the correction mode. Even if the correction processing is not performed, the extension display control unit 200 described below may perform control to display the oxygen saturation image.

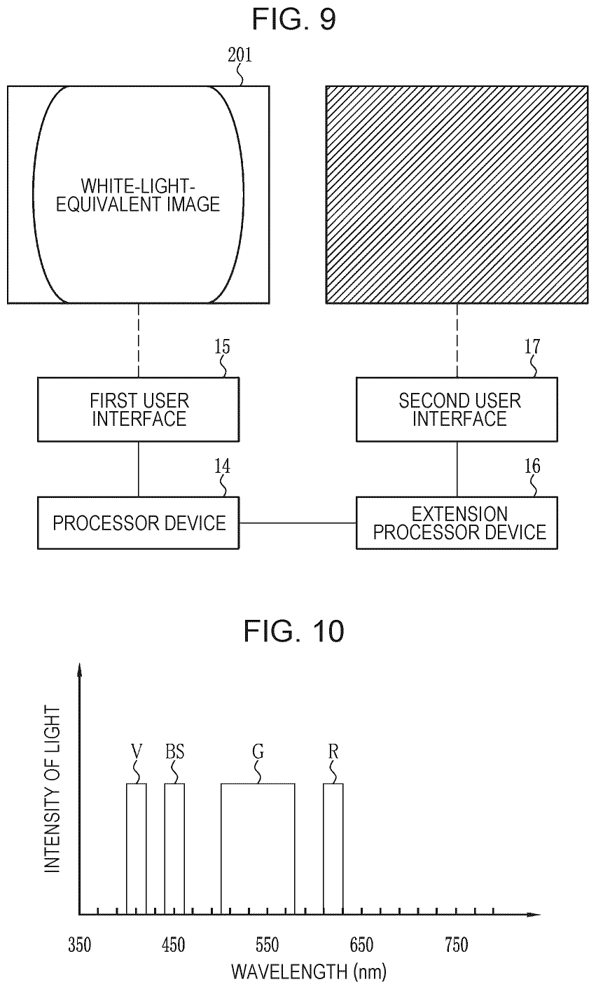

In the correction mode, as illustrated in FIG. 9, the display control unit 80 of the processor device 14 displays the white-light-equivalent image 201 on the display serving as the first user interface 15. As illustrated in FIG. 9, the display serving as the second user interface 17 displays nothing (indicated by hatching), or displays the notification image 82, a display image described below, an oxygen saturation image obtained by reflecting a corrected oxygen saturation described below in a base image described below, a correction image, or a warning display image. The image display control for the second user interface 17 is controlled by the extension display control unit 200 of the extension processor device 16.

When the mode is switched to the oxygen saturation mode without being set to the correction mode, the oxygen saturation image is not displayed, and the notification image 82 that displays the message MS0 is displayed on the display to prompt the user to switch the mode to the correction mode. When the correction processing is completed in the correction mode, the mode is switched to the oxygen saturation mode automatically or in response to a mode switching operation by the user. Alternatively, when the correction processing is completed in the correction mode, the notification image 82 that displays a message “The correction processing is completed” may be displayed to prompt the user to switch from the correction mode to the oxygen saturation mode.

The turn-on or turn-off control in each mode will be described hereinafter. First, the illumination light emitted in each mode will be described. In the normal mode according to the first embodiment, white light is emitted from the light source unit 20. The white light is broadband illumination light including the violet light V, the second blue light BS, the green light G, and the red light R emitted from the V-LED 20a, the BS-LED 20b, the G-LED 20d, and the R-LED 20e that are simultaneously turned on and having respective wavelength ranges as illustrated in FIG. 10.

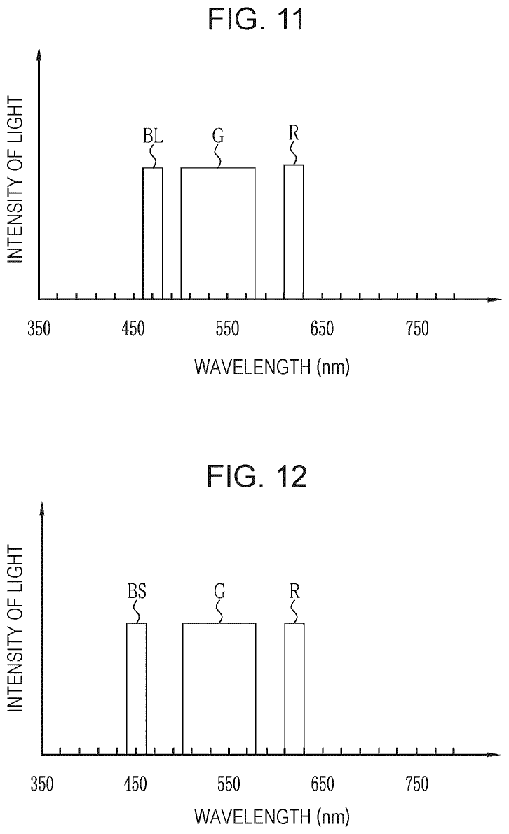

In the oxygen saturation mode according to the first embodiment, calculation illumination light (hereinafter referred to as first illumination light) and white-equivalent light (hereinafter referred to as second illumination light) are emitted from the light source unit 20. The first illumination light is broadband illumination light including the first blue light BL, the green light G, and the red light R emitted from the BL-LED 20c, the G-LED 20d, and the R-LED 20e that are simultaneously turned on and having respective wavelength ranges as illustrated in FIG. 11. The wavelength range of the first illumination light is different from that of the white light.

The second illumination light is illumination light including the second blue light BS, the green light G, and the red light R emitted from the BS-LED 20b, the G-LED 20d, and the R-LED 20e that are simultaneously turned on and having respective wavelength ranges as illustrated in FIG. 12.

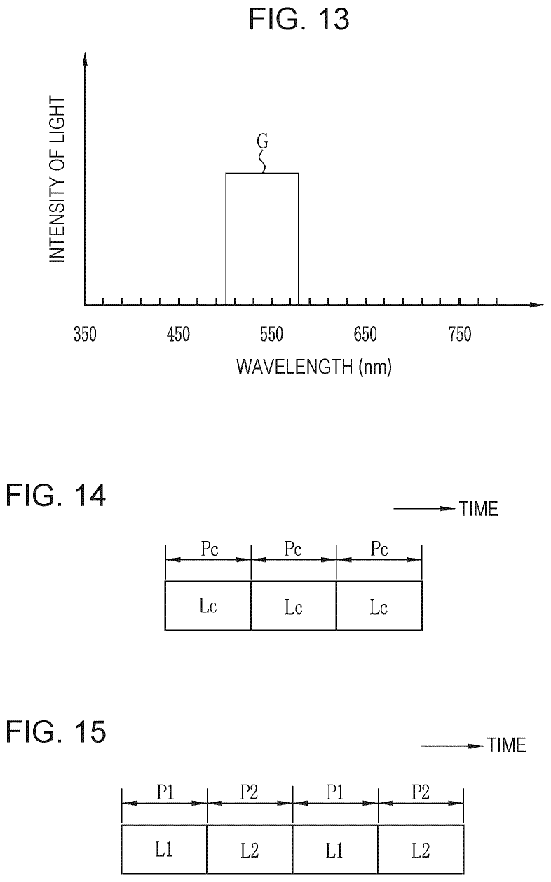

In the correction mode according to the first embodiment, the first illumination light, the second illumination light, and correction illumination light (hereinafter referred to as third illumination light) are emitted from the light source unit 20. The third illumination light is illumination light formed of the green light G emitted from the G-LED 20d that is turned on and having a wavelength range as illustrated in FIG. 13. In this specification, the term “illumination light” is used as a term meaning any one of, or collectively, the white light, the first illumination light, the second illumination light, the third illumination light, mixed light, fourth illumination light, the violet light V, the first blue light BL, the second blue light BS, the green light G, and the red light R, or as a term meaning light emitted from the light source device 13. In FIGS. 10, 11, 12, and 13, the individual intensities of the illumination light are set to a constant value for simplicity. The individual intensities of the illumination light are not limited to a constant value. The third illumination light is not limited to the light of a single color as illustrated in FIG. 13, and may be illumination light using light of a plurality of colors.

A light emission pattern in each mode will be described hereinafter. In the normal mode according to the first embodiment, each light source is controlled to be turned on or off in accordance with a normal-mode light emission pattern. The normal-mode light emission pattern is a light emission pattern as illustrated in FIG. 14 in which a pattern of emitting white light Lc over one white-light illumination period Pc is repeated. The illumination period means a certain period of time during which illumination light is turned on. One illumination period is provided for each frame. A frame is a unit of time that includes at least a time period from a timing at which illumination light is emitted to the end of reading of image signals by the imaging sensor 44.

In the oxygen saturation mode according to the first embodiment, each light source is controlled to be turned on or off in accordance with an oxygen-saturation-mode light emission pattern. The oxygen-saturation-mode light emission pattern is a light emission pattern as illustrated in FIG. 15 in which a light emission pattern of emitting first illumination light L1 over one first illumination period P1 and emitting second illumination light L2 over one second illumination period P2 is repeated.

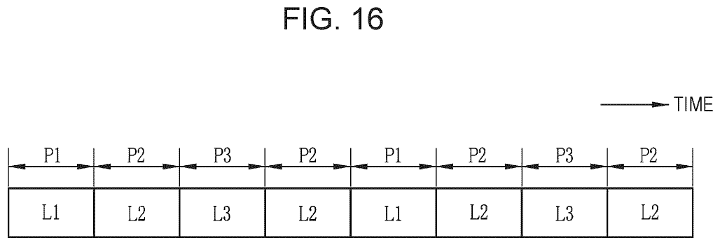

In the correction mode according to the first embodiment, each light source is controlled to be turned on or off in accordance with a correction-mode light emission pattern. The correction-mode light emission pattern is a light emission pattern as illustrated in FIG. 16 in which a light emission pattern of emitting the first illumination light L1 over one first illumination period P1, emitting the second illumination light L2 over one second illumination period P2, emitting third illumination light L3 over one third illumination period P3, and emitting the second illumination light L2 over one second illumination period P2 is repeated. That is, the correction-mode light emission pattern is a light emission pattern in which illumination light is emitted in the order of the first illumination light L1, the second illumination light L2, the third illumination light L3, the second illumination light L2, and so on.

The image signals output from the imaging sensor 44 in each mode will be described hereinafter. First, the wavelength ranges and transmittances of light transmitted through the B color filter, the G color filter, and the R color filter that the imaging sensor 44 has will be described.

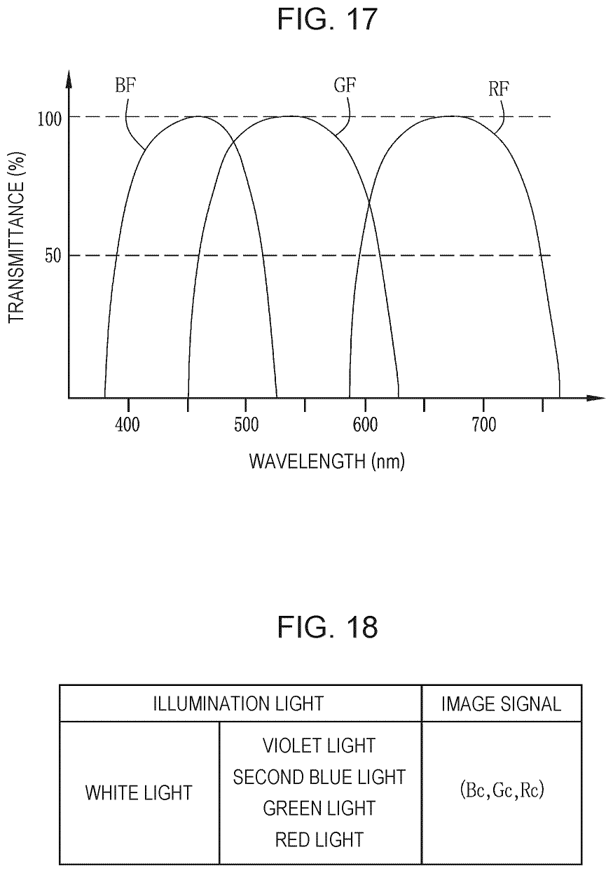

As illustrated in FIG. 17, the B pixels of the imaging sensor 44 are provided with a B color filter BF that mainly transmits light in the blue band, namely, light in a wavelength range of 380 to 560 nm (blue transmission band). A peak wavelength at which the transmittance is maximum appears around 460 to 470 nm. The G pixels of the imaging sensor 44 are provided with a G color filter GF that mainly transmits light in the green band, namely, light in a wavelength range of 450 to 630 nm (green transmission band). The R pixels of the imaging sensor 44 are provided with an R color filter RF that mainly transmits light in the red band, namely, light in the range of 580 to 760 nm (red transmission band).

The image signals output in each mode will be described hereinafter. In the normal mode according to the first embodiment, the imaging control unit 45 controls the imaging sensor 44 such that reflected light from the photographic subject illuminated with the white light is captured for each frame. With this control, in the normal mode according to the first embodiment, as illustrated in FIG. 18, a Bc image signal, a Gc image signal, and an Rc image signal are output from the B pixels, the G pixels, and the R pixels of the imaging sensor 44, respectively, for each frame including the white-light illumination period Pc over which the white light including the violet light V, the second blue light BS, the green light G, and the red light R is emitted.

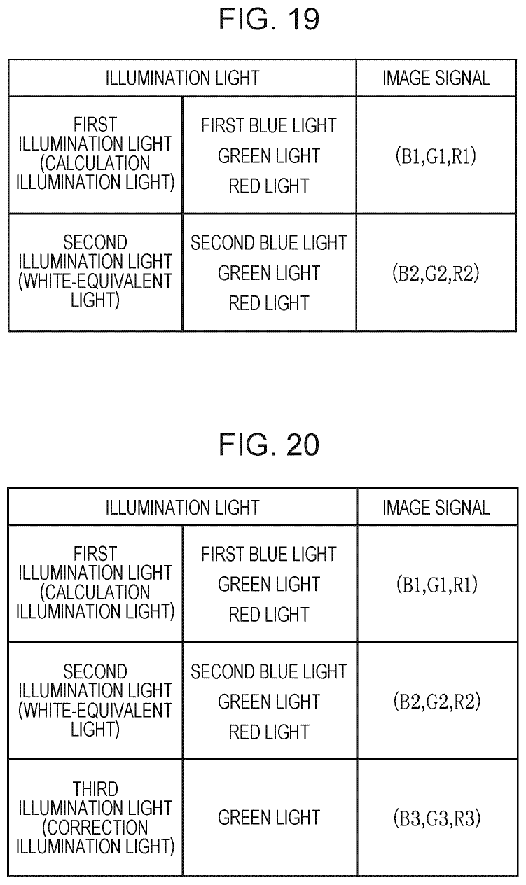

In the oxygen saturation mode according to the first embodiment, the imaging control unit 45 controls the imaging sensor 44 such that reflected light from the photographic subject illuminated with the first illumination light or the second illumination light is captured for each frame. With this control, in the oxygen saturation mode according to the first embodiment, as illustrated in FIG. 19, a B1 image signal, a G1 image signal, and an R1 image signal are output from the B pixels, the G pixels, and the R pixels of the imaging sensor 44, respectively, for each frame including the first illumination period P1 over which the first illumination light including the first blue light BL, the green light G, and the red light R is emitted. Further, a B2 image signal, a G2 image signal, and an R2 image signal are output from the B pixels, the G pixels, and the R pixels of the imaging sensor 44, respectively, for each frame including the second illumination period P2 over which the second illumination light including the second blue light BS, the green light G, and the red light R is emitted.

In the correction mode according to the first embodiment, the imaging control unit 45 controls the imaging sensor 44 such that reflected light from the photographic subject illuminated with the first illumination light, the second illumination light, or the third illumination light is captured for each frame. With this control, in the correction mode according to the first embodiment, as illustrated in FIG. 20, a B1 image signal, a G1 image signal, and an R1 image signal are output from the B pixels, the G pixels, and the R pixels of the imaging sensor 44, respectively, for each frame including the first illumination period P1 over which the first illumination light including the first blue light BL, the green light G, and the red light R is emitted. Further, a B2 image signal, a G2 image signal, and an R2 image signal are output from the B pixels, the G pixels, and the R pixels of the imaging sensor 44, respectively, for each frame including the second illumination period P2 over which the second illumination light including the second blue light BS, the green light G, and the red light R is emitted. Further, a B3 image signal, a G3 image signal, and an R3 image signal are output from the B pixels, the G pixels, and the R pixels of the imaging sensor 44, respectively, for each frame including the third illumination period P3 over which the third illumination light including the green light G is emitted. FIGS. 18, 19, and 20 illustrate the relationship between illumination light emitted over one illumination period and image signals output in a frame including one illumination period.

The characteristics of the image signals output in each mode, an oxygen saturation calculation table, and the correction processing will be described hereinafter. In the oxygen saturation mode, the B1 image signal, the G2 image signal, and the R2 image signal among the image signals output in the oxygen saturation mode are used to calculate the oxygen saturation.

In the correction mode, in consideration of the presence of a specific pigment that affects the calculation accuracy of the oxygen saturation, the B1 image signal, the G2 image signal, the R2 image signal, the B3 image signal, and the G3 image signal among the image signals output in the correction mode are used to correct the oxygen saturation calculation table.

In the oxygen saturation mode and the correction mode, the hemoglobin reflection spectrum indicating the relationship between the wavelength range of the first illumination light emitted toward a biological tissue as the observation target and the intensity of reflected light from deoxyhemoglobin (Hb) and oxyhemoglobin (HbO₂) in the biological tissue, the reflected light resulting from the reflected light obtained by illuminating the biological tissue using the first illumination light, changes depending on the blood concentration. The blood concentration means a hemoglobin concentration (amount of hemoglobin) included in a biological tissue. Deoxyhemoglobin (Hb) is hemoglobin that is not bound to oxygen (O₂). Oxyhemoglobin (HbO₂) is hemoglobin bound to oxygen (O₂).

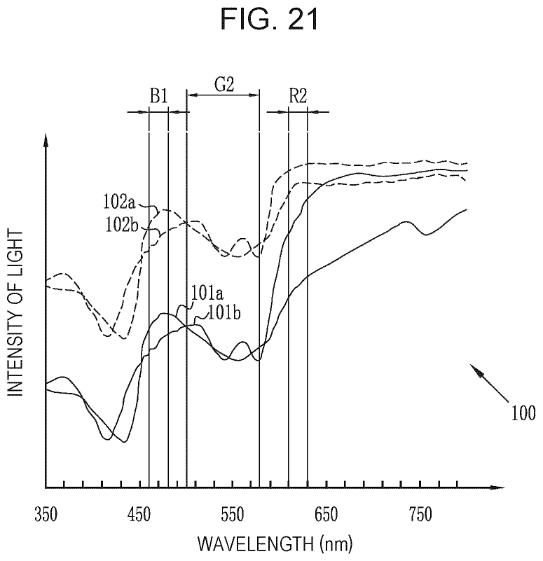

Hemoglobin reflection spectra 100 in a case where the specific pigment is not included in the biological tissue are represented by curves illustrated in FIG. 21. Of the curves illustrated in FIG. 21, curves 101a and 101b indicated by solid lines represent reflection spectra of hemoglobin when the blood concentration is high. The curve 101a represents a reflection spectrum of deoxyhemoglobin (Hb) when the blood concentration is high, and the curve 101b represents a reflection spectrum of oxyhemoglobin (HbO₂) when the blood concentration is high.

Of the curves illustrated in FIG. 21, curves 102a and 102b indicated by broken lines represent reflection spectra of hemoglobin when the blood concentration is low. The curve 102a represents a reflection spectrum of deoxyhemoglobin (Hb) when the blood concentration is low, and the curve 102b represents a reflection spectrum of oxyhemoglobin (HbO₂) when the blood concentration is low.

The B1 image signal is an image signal output from each of the B pixels in response to the imaging sensor 44 capturing the light transmitted through the B color filter BF and resulting from the reflected light reflected from the photographic subject illuminated with the first illumination light including the first blue light BL having a center wavelength of 470 nm±10 nm. Accordingly, based on the relationship between the wavelength range of the first blue light BL (see FIG. 11) and the transmission band of the B color filter BF of the imaging sensor 44 (see FIG. 17), the B1 image signal includes information on a wavelength range B1 illustrated in FIG. 21. The wavelength range B1 is a wavelength range (460 nm to 480 nm) in which the difference in reflection spectrum between deoxyhemoglobin (Hb) and oxyhemoglobin (HbO₂) is large, as indicated by the curves 101a and 101b (when the blood concentration is high) or the curves 102a and 102b (when the blood concentration is low) in FIG. 21.

The G2 image signal is an image signal output from each of the G pixels in response to the imaging sensor 44 capturing the light transmitted through the G color filter GF and resulting from the reflected light reflected from the photographic subject illuminated with the second illumination light including the green light G. Accordingly, based on the relationship between the wavelength range of the green light G (see FIG. 12) and the transmission band of the G color filter GF of the imaging sensor 44 (see FIG. 17), the G2 image signal includes information on a wavelength range G2 illustrated in FIG. 21.

The R2 image signal is an image signal output from each of the R pixels in response to the imaging sensor 44 capturing the light transmitted through the R color filter RF and resulting from the reflected light reflected from the photographic subject illuminated with the second illumination light including the red light R. Accordingly, based on the relationship between the wavelength range of the red light R (see FIG. 12) and the transmission band of the R color filter RF of the imaging sensor 44 (see FIG. 17), the R2 image signal includes information on the wavelength range R2 illustrated in FIG. 21.

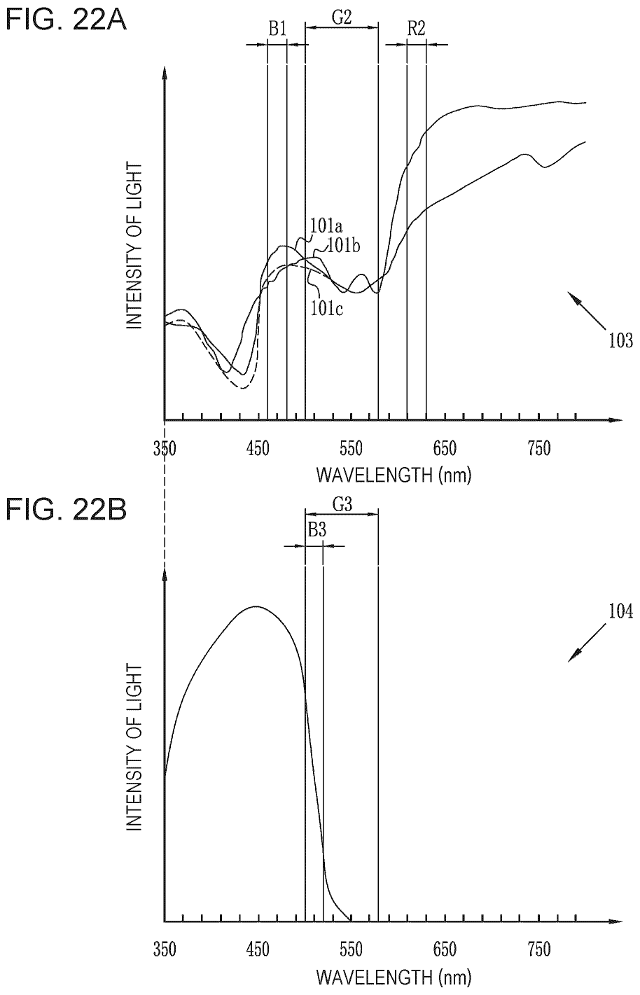

The observation target may include a specific pigment that is a pigment other than deoxyhemoglobin (Hb) or oxyhemoglobin (HbO₂) and that affects the calculation of the oxygen saturation. The specific pigment is, for example, a yellow pigment. When the specific pigment is included in the biological tissue, the absorption spectrum of the specific pigment is further considered, and thus the reflection spectrum of hemoglobin is partially different from that when the specific pigment is not included in the biological tissue (see FIG. 21). An example of hemoglobin reflection spectra 103 when the specific pigment is included in the biological tissue is illustrated in FIG. 22A.

FIG. 22A illustrates the reflection spectrum (the curve 101a) of deoxyhemoglobin (Hb) when the blood concentration is high and the reflection spectrum (the curve 101b) of oxyhemoglobin (HbO₂) when the blood concentration is high among the curves illustrated in FIG. 21. The wavelength range B1, the wavelength range G2, and the wavelength range R2 illustrated in FIG. 21 are also illustrated.

Referring to an absorption spectrum 104 of the yellow pigment illustrated in FIG. 22B, the absorption spectrum of the yellow pigment is large in the wavelength range B1 illustrated in FIG. 22A, which is a range in which the difference in reflection spectrum between deoxyhemoglobin (Hb) and oxyhemoglobin (HbO₂) is large. Accordingly, when the observation target is illuminated with the first illumination light including the first blue light BL having a center wavelength of 470 nm±10 nm, a part of the first illumination light (particularly, the first blue light BL) is absorbed by the yellow pigment.

Thus, in the presence of the yellow pigment in the biological tissue as the observation target, as indicated by a curve 101c in FIG. 22A, the reflection spectrum of deoxyhemoglobin (Hb) is lower than the reflection spectrum (the curve 101a) of deoxyhemoglobin (Hb) in the absence of the specific pigment, particularly, in the range of the wavelength range B1. As a result, the signal value of the B1 image signal decreases in the presence of the yellow pigment. A wavelength range B3 and a wavelength range G3 illustrated in FIG. 22B are ranges in which the influence of the yellow pigment on the hemoglobin reflection spectrum is smaller than that in the wavelength range B1. The wavelength range B3 and the wavelength range G3 will be described in detail below.

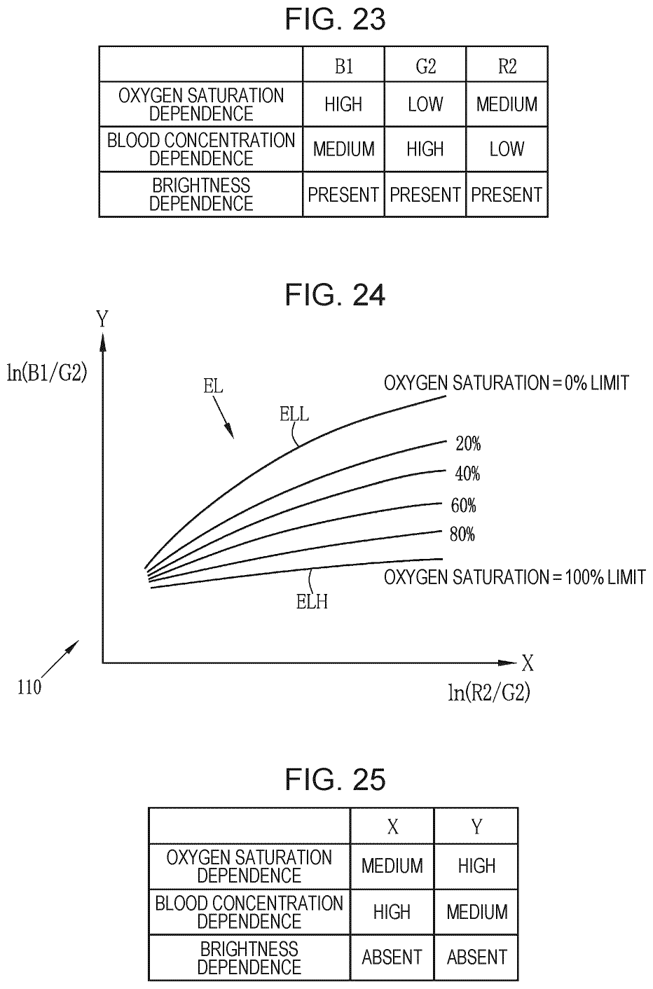

In the oxygen saturation mode and the correction mode, as illustrated in FIG. 23, the B1 image signal, the G2 image signal, and the R2 image signal, which are image signals output from the imaging sensor 44, have dependence on the oxygen saturation, the blood concentration, and the brightness. The dependence on the oxygen saturation (oxygen saturation dependence) is the degree of change in signal value (or signal ratio described below) according to the level of the oxygen saturation. In FIG. 23, the oxygen saturation dependence is qualitatively expressed as “high”, “medium”, and “low”.

The dependence on the blood concentration (blood concentration dependence) is the degree of change in signal value (or signal ratio described below) according to the level of the blood concentration. In FIG. 23, the blood concentration dependence is qualitatively expressed as “high”, “medium”, and “low”. The dependence on the brightness (brightness dependence) means whether a signal value (or signal ratio described below) changes according to the level of the brightness. In FIG. 23, the brightness dependence is represented as “present” when brightness dependence is found, and represented as “absent” when no brightness dependence is found.

As illustrated in FIG. 23, for the B1 image signal (indicated by “B1” in FIG. 23), the oxygen saturation dependence is “high”, the blood concentration dependence is “medium”, and the brightness dependence is “present”. Also, for the G2 image signal (indicated by “G2” in FIG. 23), the oxygen saturation dependence is “low”, the blood concentration dependence is “high”, and the brightness dependence is “present”. For the R2 image signal (indicated by “R2” in FIG. 23), the oxygen saturation dependence is “medium”, the blood concentration dependence is “low”, and the brightness dependence is “present”.

As described above, all of the B1 image signal, the G2 image signal, and the R2 image signal have brightness dependence. For this reason, an oxygen saturation calculation table 110 for calculating the oxygen saturation is created based on the relationship between the signal ratio ln(B1/G2) obtained by normalizing the B1 image signal by the G2 image signal and the signal ratio ln(R2/G2) obtained by normalizing the R2 image signal by the G2 image signal, with the G2 image signal being used as a normalization signal. The term “In” for the signal ratio ln(B1/G2) is a natural logarithm (the same applies to the signal ratio ln(R2/G2)).

The oxygen saturation calculation table 110 is created in advance by using the correlation between the signal ratio of image signals acquired experimentally and the oxygen saturation, and is stored in the extension processor device 16. The image signals used to generate the oxygen saturation calculation table 110 are obtained by preparing a plurality of phantoms each simulating a living body having a certain level of oxygen saturation in accordance with a plurality of levels of oxygen saturation and capturing an image of each phantom. The correlation between the signal ratio of the image signals and the oxygen saturation may be obtained in advance by simulation.

In a representation using a two-dimensional coordinate system in which the signal ratio ln(R2/G2) is on the X-axis and the signal ratio ln(B1/G2) is on the Y-axis, the correlation between the signal ratios ln(B1/G2) and ln(R2/G2) and the oxygen saturation is represented by contour lines EL on the oxygen saturation calculation table 110 as illustrated in FIG. 24. The contour lines EL are each a line drawn by connecting points (X1, Y1)=(ln(R2/G2), ln(B1/G2)) having the same oxygen saturation in the relationship between the X-component value (the signal ratio ln(R2/G2)) and the Y-component value (the signal ratio ln(B1/G2)). A contour line ELH is a contour line indicating an oxygen saturation of “100%”. A contour line ELL is a contour line indicating an oxygen saturation of “0%”. On the oxygen saturation calculation table 110 illustrated in FIG. 24, contour lines are distributed such that the oxygen saturation gradually decreases from the contour line ELH toward the contour line ELL (in FIG. 24, contour lines having oxygen saturations of “80%”, “60%”, “40%”, and “20%” are drawn).

As illustrated in FIG. 25, the X-component value (the signal ratio ln(R2/G2)) and the Y-component value (the signal ratio ln(B1/G2)) have oxygen saturation dependence and blood concentration dependence. With respect to the brightness dependence, as illustrated in FIG. 25, the X-component value and the Y-component value are normalized by the G2 image signal, and thus the brightness dependence is “absent”. For the X-component value, the oxygen saturation dependence is “medium” and the blood concentration dependence is “high”. For the Y-component value, in contrast, the oxygen saturation dependence is “high” and the blood concentration dependence is “medium”.

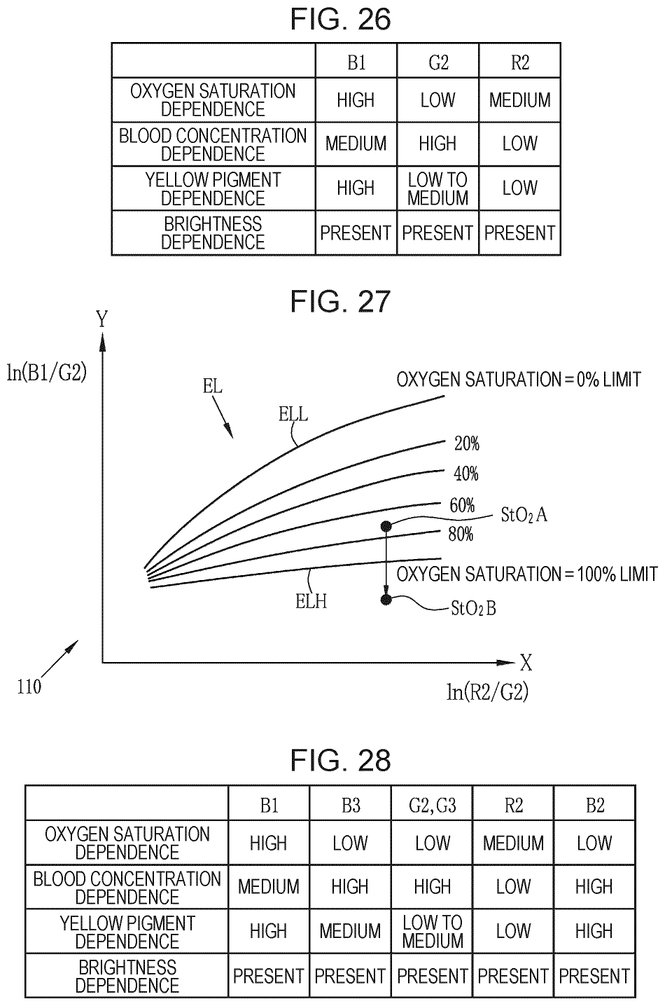

As illustrated in FIG. 26, the B1 image signal, the G2 image signal, and the R2 image signal have dependence on the yellow pigment (yellow pigment dependence) in addition to oxygen saturation dependence, blood concentration dependence, and brightness dependence. The yellow pigment dependence is the degree of change in signal value (or signal ratio) according to the level of the yellow pigment concentration. In FIG. 26, the yellow pigment dependence is qualitatively expressed as “high”, “medium”, and “low”.

FIG. 26 illustrates the yellow pigment dependence of the B1 image signal, the G2 image signal, and the R2 image signal, in addition to the oxygen saturation dependence, the blood concentration dependence, and the brightness dependence of the B1 image signal, the G2 image signal, and the R2 image signal illustrated in FIG. 23. As illustrated in FIG. 26, the yellow pigment dependence of the B1 image signal (indicated by “B1” in FIG. 26) is “high”. This is because, as illustrated in FIG. 22A, in the presence of the yellow pigment, when the reflection spectrum of deoxyhemoglobin (Hb) in the range of the wavelength range B1 becomes small, the signal value of the B1 image signal decreases. The yellow pigment dependence of the G2 image signal (indicated by “G2” in FIG. 26) is “low to medium”. The yellow pigment dependence of the R2 image signal (indicated by “R2” in FIG. 26) is “low”.

When the signal value of the B1 image signal decreases due to the presence of the yellow pigment, the Y-component value (the signal ratio ln(B1/G2)) also decreases in the calculation of the oxygen saturation using the oxygen saturation calculation table 110 as illustrated in FIG. 24. Accordingly, as illustrated in FIG. 27, in the oxygen saturation calculation table 110, due to the presence of the yellow pigment, an oxygen saturation StO₂B in the presence of the yellow pigment is calculated to be apparently higher than an oxygen saturation StO₂A in the absence of the yellow pigment.

For the reasons described above, when the yellow pigment is present, it is preferable to perform correction processing for calculating a more appropriate oxygen saturation in accordance with the concentration of the yellow pigment. In the correction mode, accordingly, the B3 image signal and the G3 image signal are further acquired by capturing reflected light obtained by illuminating the photographic subject with the third illumination light.

The B3 image signal is an image signal output from each of the B pixels in response to the imaging sensor 44 capturing the light transmitted through the B color filter BF and resulting from the reflected light obtained by illuminating the photographic subject with the third illumination light. Accordingly, based on the relationship between the wavelength range of the green light G (see FIG. 13) and the transmission band of the B color filter BF of the imaging sensor 44 (see FIG. 17), the B3 image signal includes information on the wavelength range B3 illustrated in FIG. 22B.

The G3 image signal is an image signal output from each of the G pixels in response to the imaging sensor 44 capturing the light transmitted through the G color filter GF and resulting from the reflected light obtained by illuminating the photographic subject with the third illumination light composed of the green light G. Accordingly, based on the relationship between the wavelength range of the green light G (see FIG. 13) and the transmission band of the G color filter GF of the imaging sensor 44 (see FIG. 17), the G3 image signal includes information on the wavelength range G3 illustrated in FIG. 22B.

As illustrated in FIG. 28, like the B1 image signal, the G2 image signal, and the R2 image signal, the B3 image signal and the G3 image signal have yellow pigment dependence in addition to oxygen saturation dependence, blood concentration dependence, and brightness dependence. As illustrated in FIG. 28, for the B3 image signal (indicated by “B3” in FIG. 28), the oxygen saturation dependence is “low”, the blood concentration dependence is “high”, the yellow pigment dependence is “medium”, and the brightness dependence is “present”. For the G3 image signal (indicated by “G3” in FIG. 28), as for the G2 image signal, the oxygen saturation dependence is “low”, the blood concentration dependence is “high”, the yellow pigment dependence is “low to medium”, and the brightness dependence is “present”.

As illustrated in FIG. 28, since the yellow pigment dependence of the B2 image signal is also “high”, the B2 image signal may be used instead of the B3 image signal in the correction processing. As illustrated in FIG. 28, for the B2 image signal, the oxygen saturation dependence is “low”, the blood concentration dependence is “high”, and the brightness dependence is “present”.

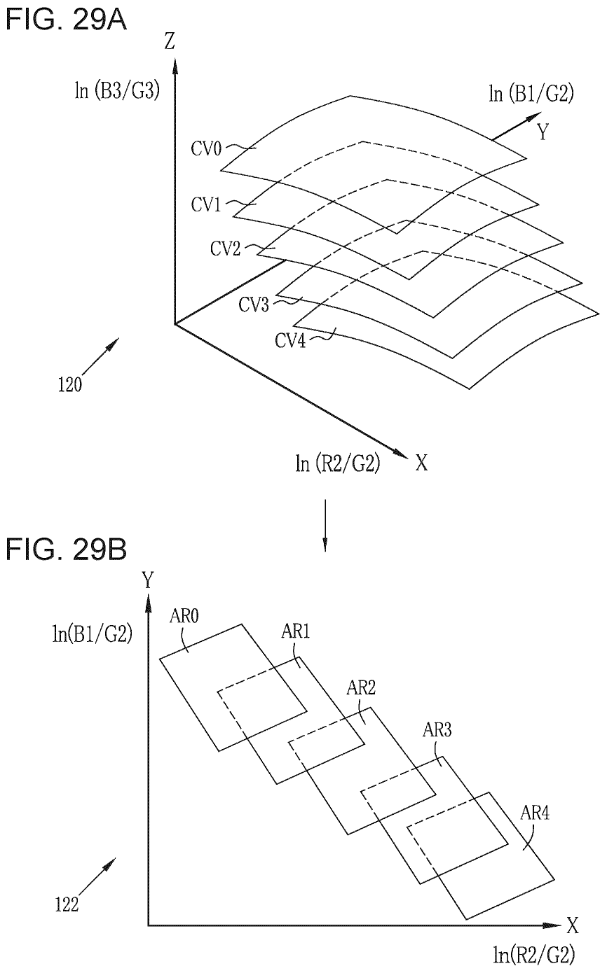

A corrected oxygen saturation calculation table 120 as illustrated in FIG. 29A is used for the calculation of the oxygen saturation in consideration of the concentration of the specific pigment. The corrected oxygen saturation calculation table 120 is a table representing the correlation among the signal ratio ln(B1/G2), the signal ratio ln(R2/G2), the signal ratio ln(B3/G3), and the oxygen saturation corresponding to the concentration of the specific pigment. The signal ratio ln(B3/G3) is a signal ratio obtained by normalizing the B3 image signal by the G3 image signal. Like the oxygen saturation calculation table 110, the corrected oxygen saturation calculation table 120 is created in advance and stored in the extension processor device 16.

In the corrected oxygen saturation calculation table 120, as illustrated in FIG. 29A, curved surfaces CV0 to CV4 representing levels of oxygen saturation are distributed in the Z-axis direction on a three-dimensional coordinate system in which the signal ratio ln(R2/G2) is on the X-axis, the signal ratio ln(B1/G2) is on the Y-axis, and the signal ratio ln(B3/G3) is on the Z-axis in accordance with the concentration of the yellow pigment (hereinafter referred to as a first pigment value). The curved surface CV0 represents the level of oxygen saturation in a case where the first pigment value is “0” (a case where the yellow pigment is not present or a case where the yellow pigment is present but does not affect the calculation of the oxygen saturation due to the amount thereof being very small). The curved surfaces CV1 to CV4 represent the levels of oxygen saturation in a case where the first pigment values are “1” to “4”, respectively. The larger the first pigment value, the higher the concentration of the yellow pigment. As indicated by the curved surfaces CV0 to CV4, as the first pigment value increases, the value of the Z-axis changes in a decreasing direction.

As illustrated in FIG. 29A, when the levels of oxygen saturation (the curved surfaces CV0 to CV4) corresponding to the respective first pigment values, which are expressed in a three-dimensional coordinate system in which the signal ratio ln(R2/G2) is on the X-axis, the signal ratio ln(B1/G2) is on the Y-axis, and the signal ratio ln(B3/G3) is on the Z-axis, are expressed in a two-dimensional coordinate system 122 in which the signal ratio ln(R2/G2) is on the X-axis and the signal ratio ln(B1/G2) is on the Y-axis, as illustrated in FIG. 29B, areas AR0 to AR4 representing the levels of oxygen saturation are distributed at different positions according to the respective first pigment values. The areas AR0 to AR4 represent the distributions of oxygen saturation in a case where the first pigment values are “0” to “4”, respectively. The contour lines EL (see FIG. 24) representing the levels of oxygen saturation for the areas AR0 to AR4 are determined. As a result, the oxygen saturation corresponding to the concentration of the yellow pigment can be obtained. As indicated by the areas AR0 to AR4, as the first pigment value increases, the value of the X-axis increases and the value of the Y-axis decreases.

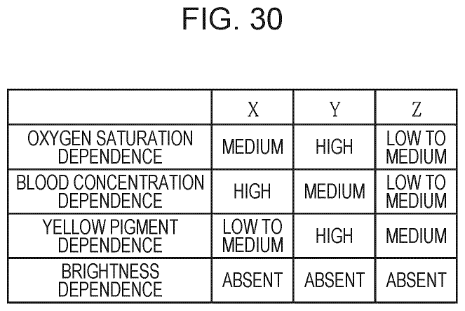

As illustrated in FIG. 30, the X-component value (the signal ratio ln(R2/G2)), the Y-component value (the signal ratio ln(B1/G2)), and the Z-component value (the signal ratio ln(B3/G3)) have oxygen saturation dependence, blood concentration dependence, and yellow pigment dependence. The yellow pigment dependence of the X-component value is “low to medium”, the yellow pigment dependence of the Y-component value is “high”, and the yellow pigment dependence of the Z-component value is “medium”. For the Z-component value, the oxygen saturation dependence is “low to medium” and the blood concentration dependence is “low to medium”. Since the X-component value and the Y-component value are normalized by the G2 image signal and the Z-component value is normalized by the G3 image signal, the brightness dependence is “absent”.

That is, the “correction processing” in the correction mode according to the first embodiment is a process of further acquiring, in addition to the image signals acquired in the oxygen saturation mode, image signals having yellow pigment dependence and having oxygen saturation dependence and blood concentration dependence that are different from each other, referring to the corrected oxygen saturation calculation table 120 represented by the three-dimensional coordinate system, and selecting an oxygen saturation calculation table corresponding to a specific pigment concentration. Switching to the oxygen saturation mode again after the correction processing is completed allows a more accurate oxygen saturation to be calculated using the oxygen saturation calculation table corresponding to the specific pigment concentration of the tissue during observation.

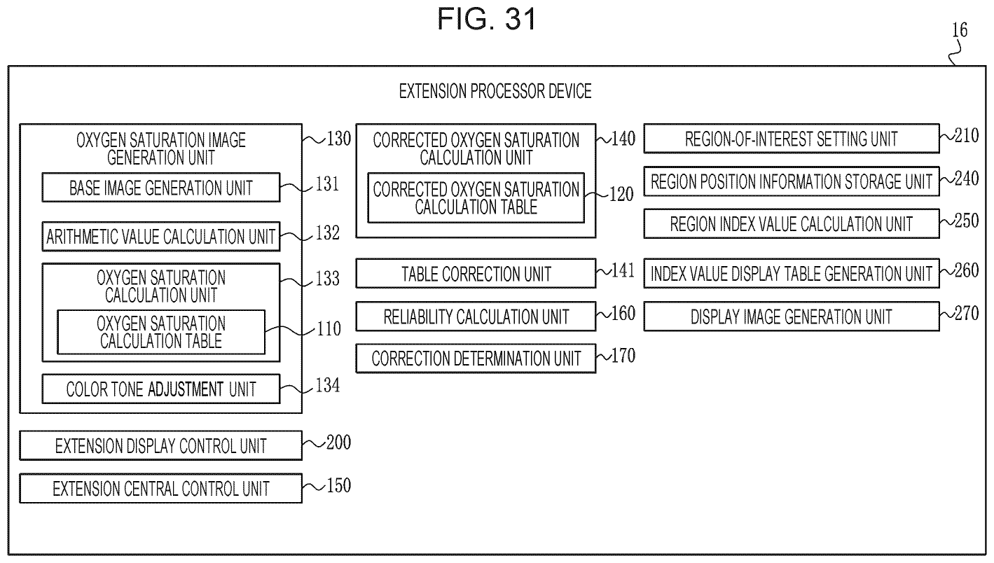

As illustrated in FIG. 31, the extension processor device 16 includes an oxygen saturation image generation unit 130, a corrected oxygen saturation calculation unit 140, a table correction unit 141, an extension central control unit 150, a reliability calculation unit 160, a correction determination unit 170, an extension display control unit 200, a region-of-interest setting unit 210, a region position information storage unit 240, a region index value calculation unit 250, an index value display table generation unit 260, and a display image generation unit 270. In the extension processor device 16, programs related to various types of processing are incorporated in a program memory (not illustrated). The extension central control unit 150, which is constituted by a processor, executes a program in the program memory, thereby implementing functions of the oxygen saturation image generation unit 130, the corrected oxygen saturation calculation unit 140, the table correction unit 141, the reliability calculation unit 160, the correction determination unit 170, the extension display control unit 200, the region-of-interest setting unit 210, the region position information storage unit 240, the region index value calculation unit 250, the index value display table generation unit 260, and the display image generation unit 270.

The oxygen saturation image generation unit 130 has a base image generation unit 131, an arithmetic value calculation unit 132, an oxygen saturation calculation unit 133, and a color tone adjustment unit 134. The base image generation unit 131 generates a base image on the basis of the image signals transmitted from the image communication unit 90 of the processor device 14. The base image may be an image from which morphological information such as the shape of the observation target can be grasped. For example, the base image is a white-light-equivalent image generated using the B2 image signal, the G2 image signal, and the R2 image signal. The base image may be a narrowband light image that is obtained by illuminating the photographic subject with narrowband light and in which a blood vessel, a gland duct structure, and the like are highlighted.

The arithmetic value calculation unit 132 calculates arithmetic values by arithmetic processing based on the image signals transmitted from the image communication unit 90. Specifically, the arithmetic value calculation unit 132 calculates a signal ratio B1/G2 between the B1 image signal and the G2 image signal and a signal ratio R2/G2 between the R2 image signal and the G2 image signal as arithmetic values to be used for the calculation of the oxygen saturation. In the correction mode, the arithmetic value calculation unit 132 calculates a signal ratio B3/G3 between the B3 image signal and the G3 image signal. The signal ratio B1/G2, the signal ratio R2/G2, and the signal ratio B3/G3 may be in logarithmic form (ln). Alternatively, color difference signals Cr and Cb, a saturation S, a hue H, or the like converted and calculated using the B1 image signal, the G2 image signal, the R2 image signal, the B3 image signal, or the G3 image signal may be used as the arithmetic values.

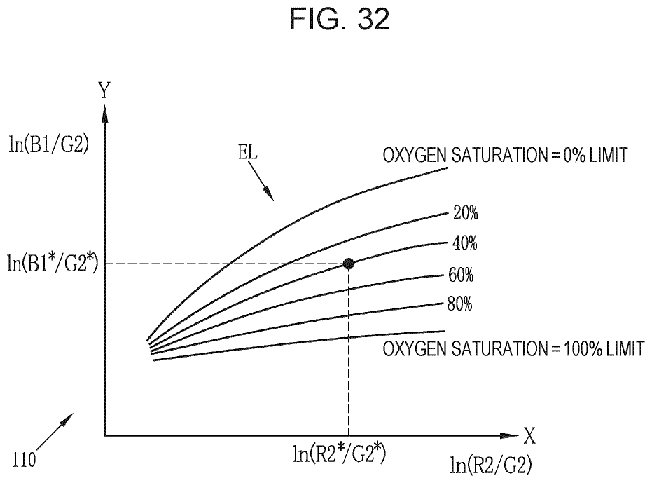

The oxygen saturation calculation unit 133 refers to the oxygen saturation calculation table 110 (see FIG. 24) by using the arithmetic values to calculate the oxygen saturation. The oxygen saturation calculation unit 133 refers to the oxygen saturation calculation table 110 and calculates, for each pixel, the oxygen saturation corresponding to the signal ratios B1/G2 and R2/G2. For example, as illustrated in FIG. 32, when a specific pixel has signal ratios ln(B1*/G2*) and ln(R2*/G2*), the oxygen saturation corresponding to the signal ratios ln(B1*/G2*) and ln(R2*/G2*) is “40%”. In this case, the oxygen saturation calculation unit 133 calculates the oxygen saturation of the specific pixel as “40%”. The oxygen saturation is one of the biological index values described below, which are values indicating the state of the observation target as the photographic subject.

The color tone adjustment unit 134 performs color tone adjustment processing using the oxygen saturation calculated by the oxygen saturation calculation unit 133 to generate an oxygen saturation image. As the color tone adjustment processing, for example, pseudo-color processing in which a color corresponding to the oxygen saturation is assigned is performed to generate an oxygen saturation image. The pseudo-color processing does not require the base image.

In another specific example of the color tone adjustment processing, an oxygen saturation image generation threshold value is set in advance, and processing is performed on the base image such that the color tone is maintained for pixels with oxygen saturations greater than or equal to the oxygen saturation image generation threshold value and, for pixels with oxygen saturations less than the oxygen saturation image generation threshold value, the color tone is changed according to the oxygen saturations, to generate an oxygen saturation image. When this color tone adjustment processing is performed, the color tone of a region having a relatively high oxygen saturation (greater than or equal to the oxygen saturation image generation threshold value) is maintained, whereas the color tone of a region having a relatively low oxygen saturation (less than the oxygen saturation image generation threshold value) is changed. Thus, for example, a site with low oxygen saturation can be recognized in a situation in which the morphological information of a site with high oxygen saturation can be observed.

After surgery, usually, tissue union occurs at the incision and suture site, leading to cure. However, incomplete tissue union at the suture site for some reason may cause suture failure in which a part or the whole of the suture site is dissociated again. Suture failure is known to occur in a region of low oxygen saturation or congestion. Accordingly, the display of the oxygen saturation image can support the user in determining an excision site or an anastomosis site at which suture failure is less likely to occur after surgery. Further, an index value display table described below and the oxygen saturation image are displayed side by side to present the real number value of the oxygen saturation to the user in addition to the representation of the oxygen saturation by the color tone, which can support the user in more accurately and easily identifying a region suitable for the incision or anastomosis.

The corrected oxygen saturation calculation unit 140 refers to the corrected oxygen saturation calculation table 120 (see FIG. 29A) by using the arithmetic values to perform correction processing. Among the arithmetic values, the signal ratio B3/G3 may be calculated and logarithmized by the corrected oxygen saturation calculation unit 140.

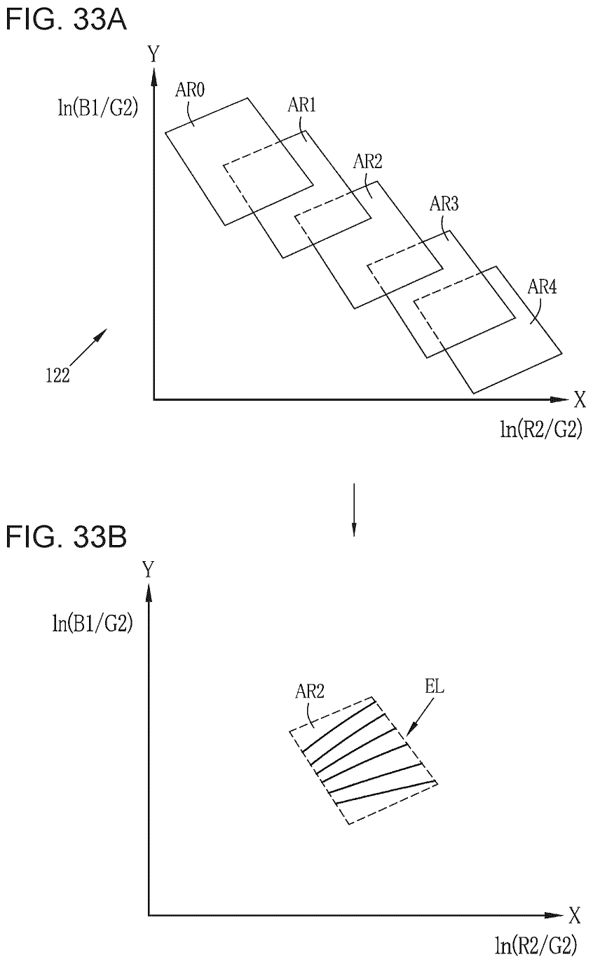

The table correction unit 141 performs, as the correction processing to be performed in the correction mode, table correction processing for setting the oxygen saturation calculation table as an oxygen saturation calculation table selected by referring to the corrected oxygen saturation calculation table 120. In the table correction processing, for example, in a case where the first pigment value is “2”, among the areas AR0 to AR4 (the curved surfaces CV0 to CV4 of the corrected oxygen saturation calculation table 120) determined according to the first pigment values illustrated in FIG. 33A, the oxygen saturation calculation table that is the area AR2 corresponding to the first pigment value “2” in which the contour lines EL as illustrated in FIG. 33B are drawn is selected. The table correction unit 141 corrects the oxygen saturation calculation table 110 such that the oxygen saturation calculation table 110 referred to by the oxygen saturation calculation unit 133 is set as the oxygen saturation calculation table that is the area AR2.

The correction processing may be performed for each pixel of the endoscopic image or may be performed for each pixel of a specific region described below. In addition, in the specific region described below, the X-component value (the signal ratio ln(R2/G2)) the Y-component value (the signal ratio ln(B1/G2)), and the Z-component value (the signal ratio ln(B3/G3)) may be calculated based on a mean signal value that is a mean value of signal values calculated for the respective pixels to perform the correction processing.

In the correction mode, instead of the correction processing using the corrected oxygen saturation calculation table 120, a corrected oxygen saturation calculation table that stores in advance the correlation between the oxygen saturation and values of a three-dimensional coordinate system in which the signal ratio ln(R2/G2) is on the X-axis, the signal ratio ln(B1/G2) is on the Y-axis, and the signal ratio ln(B3/G3) is on the Z-axis, namely, the X-component value (the signal ratio ln(R2/G2)), the Y-component value (the signal ratio ln(B1/G2)), and the Z-component value (the signal ratio ln(B3/G3)), may be used to determine a corrected oxygen saturation in consideration of the influence of the specific pigment. In this corrected oxygen saturation calculation table, a contour line or a space indicating the same oxygen saturation is distributed in three dimensions using the three-dimensional coordinate system.

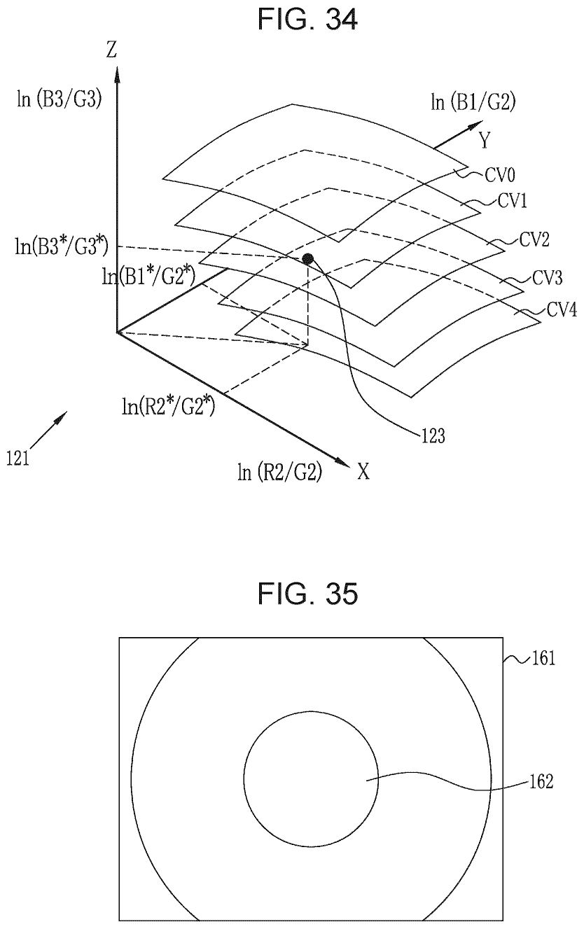

Specifically, for example, in a three-dimensional coordinate system 121 as illustrated in FIG. 34, in a case where the signal ratios of specific pixels calculated based on the B1 image signal, the G2 image signal, the R2 image signal, the B3 image signal, and the G3 image signal are signal ratios ln(R2*/G2*), ln(B1*/G2*), and ln(B3*/G3*), the corrected oxygen saturation at a point 123 corresponding to the signal ratios ln(R2*/G2*), ln(B1*/G2*), and ln(B3*/G3*) is calculated as the oxygen saturation.

Alternatively, some of the image signals obtained in the correction mode may be used to perform the correction processing in the correction mode. Some of the image signals are image signals in the specific region in a correction image described below. In this case, the specific region may be a region less influenced by disturbances that influence the calculation accuracy of the oxygen saturation. The degree of influence of disturbances on the specific region is determined by calculating the reliability of the image signals in the specific region.

Further, preprocessing for the calculation of reliability involves determining whether a pixel included in the specific region is an effective pixel. The determination of whether a pixel is an effective pixel is performed by the extension central control unit 150 of the extension processor device 16 setting a channel lower limit threshold value and a channel upper limit threshold value for each channel (a B channel, a G channel, and an R channel) of each pixel. A pixel value for which all the channels are within ranges greater than or equal to the channel lower limit values and less than the channel upper limit values of the respective colors is determined as an effective pixel, and is set as a target pixel for calculating reliability.

The disturbances refer to factors that may cause a decrease in the calculation accuracy of the oxygen saturation, other than the specific pigment, for the observation target appearing in the endoscopic image captured by the endoscope 12, such as halation, a dark portion, bleeding, fat, and deposits on a mucosal surface. The halation and the dark portion are related to the brightness of the endoscopic image. The halation means that a region of an image becomes bright, resulting in a loss of detail, due to strong light incident on the imaging sensor 44. The dark portion is a dark region of an image caused by illumination light being blocked by a structure in the living body, such as a fold or a flexure of the colon, a treatment tool, or the like, or due to the distal portion of the lumen where illumination light is difficult to reach.

Bleeding includes external bleeding outside the serosa (into the abdominal cavity) or into the gastrointestinal lumen, and internal bleeding within the mucosa. Fat includes fat observed outside the serosa (within the abdominal cavity) such as the greater omentum, the lesser omentum, and the mesentery, and fat observed on the mucosal surface of the gastrointestinal lumen. The deposits on the mucosal surface include endogenously derived deposits such as mucus, blood, and exudate, exogenously derived deposits such as a staining solution and water supplied from a water supply device, and a mixture of endogenously and exogenously derived deposits that is a residual liquid or residue.

In the correction mode, when the correction processing is performed using the image signals in the specific region, first, a correction image 161 as illustrated in FIG. 35 is displayed on the display at the timing of switching to the correction mode. The display of the correction image 161 is controlled by the extension display control unit 200. The correction image 161 displays a specific region 162 in a manner visually recognizable to the user.

The shape of the specific region 162 is not limited to a circular shape as illustrated in FIG. 35. Further, the position of the specific region 162 is not limited to the central portion of the image as illustrated in FIG. 35. For example, a region having a doughnut shape in which the peripheral portion of the correction image 161, which is greatly affected by distortion caused by the curvature of the lens, is not present and in which the central portion of the correction image 161, which is a dark portion at the distal portion of the lumen, is not present may be set as a specific region. The correction image may be a color image (for example, a white-light-equivalent image) generated using the B2 image signal, the G2 image signal, and the R2 image signal. The correction image may be an image generated using another image signal.

In a case where the correction image 161 as illustrated in FIG. 35 is displayed on the display, in response to an input of a reliability calculation instruction, the reliability calculation unit 160 of the extension processor device 16 calculates the reliability for each pixel included in the specific region 162 on the basis of the image signals in the specific region 162. The reliability calculation instruction may be input in accordance with an input instruction through a user interface or may be automatically input at the same timing as the timing of control for the display of the correction image 161.

The calculation of reliability will be described hereinafter. Examples of reliability include (1) reliability regarding the brightness of the endoscopic image, (2) reliability based on the degree of bleeding included in the endoscopic image, and (3) reliability based on the degree of fat included in the endoscopic image.

The calculation of the reliability regarding brightness will be described. In this case, the reliability calculation unit 160 refers to a first reliability calculation table 163 as illustrated in FIG. 36 using the G1 image signal to calculate the reliability. The first reliability calculation table 163 is a table generated in advance and indicating the relationship between the signal value of the G1 image signal and the reliability. The signal value of the G1 image signal is, for example, a brightness value obtained by performing a conversion process using the G1 image signal. In this case, the reliability is calculated as a value from 0 to 1. In the first reliability calculation table 163, as illustrated in FIG. 36, the reliability with respect to a signal value of the G1 image signal outside a certain range Rx is lower than the reliability with respect to a signal value of the G1 image signal within the certain range Rx. Specifically, the case where the signal value of the G1 image signal is outside the certain range Rx is, for example, a case where the brightness value is high because the pixel includes the halation or a case where the brightness value is local minimum because the specific region includes the dark portion. The reliability regarding brightness may be calculated using the G2 image signal (see FIG. 102 described below) instead of the G1 image signal.

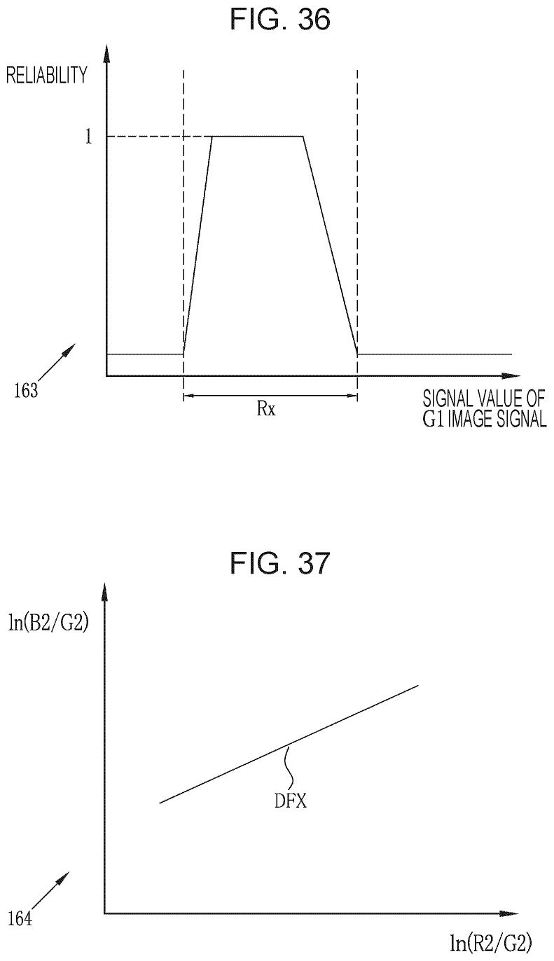

The calculation of the reliability based on the degree of bleeding will be described. In this case, the reliability calculation unit 160 refers to a second reliability calculation table 164 as illustrated in FIG. 37 using the signal ratio ln(R2/G2) and the signal ratio ln(B2/G2) to calculate the reliability. In the second reliability calculation table 164, a definition line DFX is plotted in a two-dimensional coordinate system with the X-axis representing the signal ratio ln(R2/G2) and the Y-axis representing the signal ratio ln(B2/G2). In this case, the reliability is calculated to be lower as the coordinates (X2, Y2)=(ln(R2/G2), ln(B2/G2)) calculated by setting the X-component value as the signal ratio ln(R2/G2) and the Y-component value as the signal ratio ln(B2/G2) are located at lower right in the second reliability calculation table 164. When the coordinates (X2, Y2) are located in an upper left region with respect to the definition line DFX, the reliability based on the degree of bleeding is set to a fixed value indicating high reliability. The signal ratio ln(R2/G2) is a value obtained by normalizing the R2 image signal by the G2 image signal and representing the resulting value in logarithmic form. The signal ratio ln(B2/G2) is a value obtained by normalizing the B2 image signal by the G2 image signal and representing the resulting value in logarithmic form.

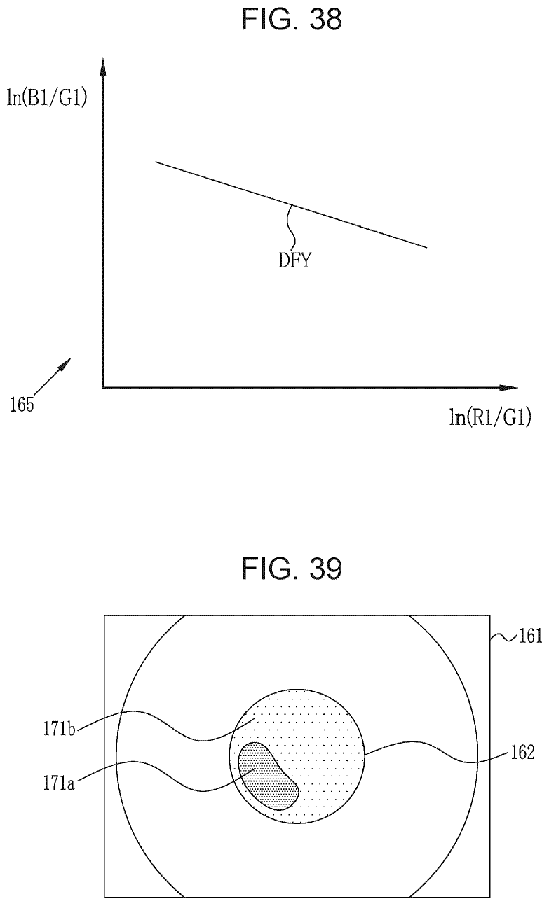

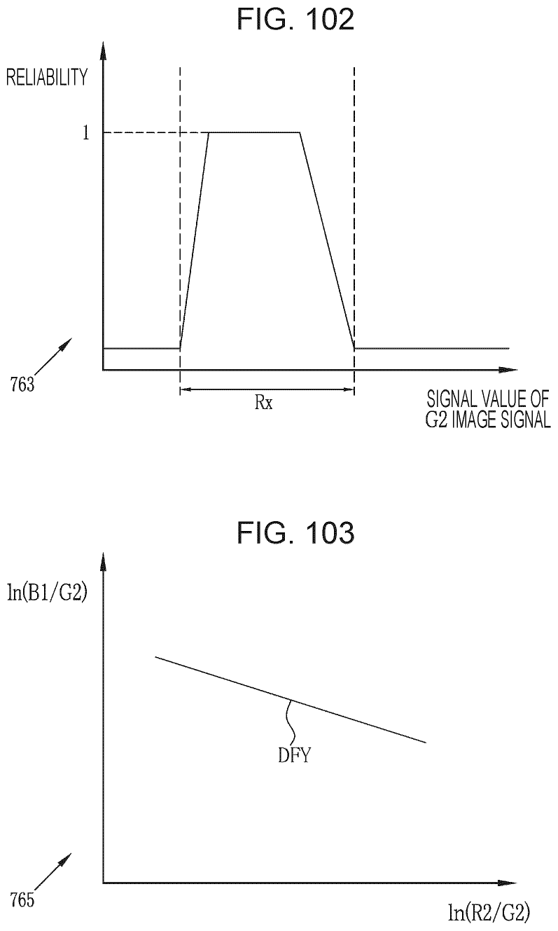

The calculation of the reliability based on the degree of fat will be described. In this case, the reliability calculation unit 160 refers to a third reliability calculation table 165 as illustrated in FIG. 38 using the signal ratio ln(R1/G1) and the signal ratio ln(B1/G1) to calculate the reliability. In the third reliability calculation table 165, a definition line DFY is plotted in a two-dimensional coordinate system with the X-axis representing the signal ratio ln(R1/G1) and the Y-axis representing the signal ratio ln(B1/G1). In this case, the reliability is calculated to be lower as the coordinates (X3, Y3)=(ln(B1/G1), ln(B1/G1)) calculated by setting the X-component value as the signal ratio ln(R1/G1) and the Y-component value as the signal ratio ln(B1/G1) are located at lower left in the third reliability calculation table 165. When the coordinates (X3, Y3) are located in an upper right region with respect to the definition line DFY, the reliability based on the degree of fat is set to a fixed value indicating high reliability. The signal ratio ln(R1/G1) is a value obtained by normalizing the R1 image signal by the G1 image signal and representing the resulting value in logarithmic form. The signal ratio ln(B1/G1) is a value obtained by normalizing the B1 image signal by the G1 image signal and representing the resulting value in logarithmic form. Alternatively, the G2 image signal may be used instead of the G1 image signal and the R2 image signal may be used instead of the R1 image signal to calculate the reliability based on the degree of fat on the basis of the position, in the reliability calculation table, of the coordinates (X3, Y3)=(ln(R2/G2), ln(B1/G2)) calculated by setting the X-component value as the signal value ln(R2/G2) and the Y-component value as the signal ratio ln(B1/G2) (see FIG. 103 described below).

The reliability calculation unit 160 calculates at least one of the reliability regarding brightness (first reliability), the reliability based on the degree of bleeding (second reliability), or the reliability based on the degree of fat (third reliability). The calculated reliability is used for notification for presenting a low-reliability region from falling within the specific region or for weighting processing for the signal values of the image signals used for the correction processing.

In the notification for presenting a low-reliability region from falling within the specific region, the calculated reliability is transmitted to the correction determination unit 170. The correction determination unit 170 uses a preset reliability determination threshold value to make a determination on the reliability calculated for each pixel in the specific region, and outputs a determination result indicating whether each pixel is a high-reliability pixel or a low-reliability pixel.

For example, the correction determination unit 170 determines that a pixel having a reliability greater than or equal to the reliability determination threshold value is a high-reliability pixel, and determines that a pixel having a reliability less than the reliability determination threshold value is a low-reliability pixel. The correction determination unit 170 transmits the determination result obtained by determining the reliability of each pixel to the extension display control unit 200. The extension display control unit 200 performs control to change the display mode of the correction image 161 displayed on the display in accordance with the determination result.

For example, as illustrated in FIG. 39, the extension display control unit 200 sets the saturation of a low-reliability region 171a to be higher than the saturation of a high-reliability region 171b in the specific region 162. The low-reliability region is a set of pixels having low-reliability pixels. The high-reliability region is a set of pixels having high-reliability pixels. The display of the correction image 161 as illustrated in FIG. 39 in which the low-reliability region 171a and the high-reliability region 171b are displayed in different modes can notify the user that a low-reliability region including a relatively large number of disturbances is included in the specific region. The method for displaying the low-reliability region 171a and the high-reliability region 171b in different modes is not limited to changing the saturation for each area. For example, the luminance, the color tone, or the like may be changed.

In the calculation of a plurality of reliabilities among the first reliability, the second reliability, and the third reliability, the lowest one of the first reliability, the second reliability, and the third reliability may be used as the reliability used for the determination on the reliability. Alternatively, a reliability determination threshold value may be set for each reliability. For example, a first reliability determination threshold value for the first reliability, a second reliability determination threshold value for the second reliability, and a third reliability determination threshold value for the third reliability may be set in advance, and if any one of the reliabilities is less than the corresponding reliability determination threshold value, the pixel for which the reliability is calculated may be determined to be a low-reliability pixel.

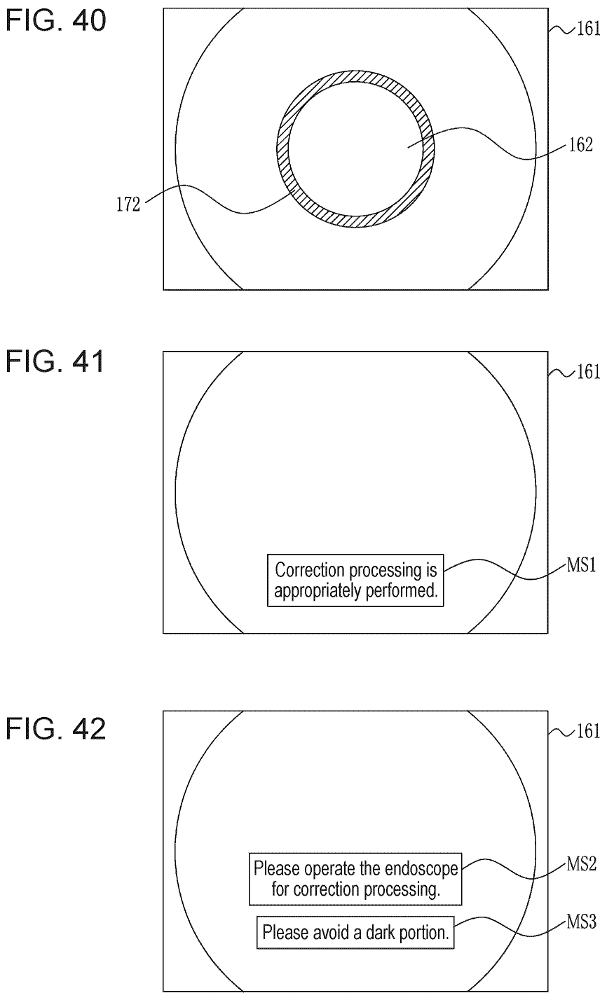

The correction determination unit 170 may further make a determination on the number of high-reliability pixels for the reliability calculated for each pixel. In this case, the extension display control unit 200 changes the display mode of the specific region depending on whether the number of high-reliability pixels in the specific region is greater than or equal to a high-reliability pixel number determination threshold value or less than the high-reliability pixel number determination threshold value. For example, when the number of high-reliability pixels in the specific region is greater than or equal to the high-reliability pixel number determination threshold value, as illustrated in FIG. 40, the correction image 161 is displayed such that the specific region is highlighted by being surrounded by a frame 172 of a first determination result color. The specific region highlighted by being surrounded by the frame of the first determination result color can notify the user that the correction processing can be performed in a state less influenced by disturbances.

When the number of high-reliability pixels in the specific region is less than the high-reliability pixel number determination threshold value, the correction image 161 may be displayed such that the specific region is highlighted by being surrounded by a frame of a second determination result color different from the first determination result color. The specific region highlighted by being surrounded by the frame of the second determination result color can notify the user that the number of pixels less influenced by disturbances is smaller than a certain value.

In response to the correction determination unit 170 making a determination on the number of low-reliability pixels, the extension display control unit 200 may change the display mode of the specific region depending on whether the number of low-reliability pixels in the specific region is greater than or equal to a low-reliability pixel number determination threshold value or less than the low-reliability pixel number determination threshold value. As described above, a reliability pixel number determination threshold value (the high-reliability pixel number determination threshold value or the low-reliability pixel number determination threshold value) is used, and the display mode of the correction image is changed according to the number of pixels having high or low reliability. As a result, the user can be notified of the degree to which disturbances are included in the specific region, and can be prompted to operate the endoscope for appropriately performing the correction processing.

Alternatively, the correction determination unit 170 may determine the reliability for each pixel in the specific region using the reliability determination threshold value and/or the reliability pixel number determination threshold value, and if it is determined that the specific region is less influenced by disturbances, a message indicating that the correction processing can be appropriately performed may be displayed in the correction image 161. For example, as illustrated in FIG. 41, a message MS1 such as “Correction processing is appropriately performed” is displayed in a superimposed manner on the correction image 161.

Alternatively, the correction determination unit 170 may determine the reliability for each pixel in the specific region using the reliability determination threshold value and/or the reliability pixel number determination threshold value, and if the specific region includes a low-reliability region or if the number of low-reliability pixels included in the specific region is greater than or equal to the reliability pixel number determination threshold value, a warning may be displayed. For example, as illustrated in FIG. 42, a message MS2 such as “Please operate the endoscope for correction processing” is displayed in a superimposed manner on the correction image 161. If it is determined that the reliability regarding brightness has a particularly large influence, as illustrated in FIG. 42, a message MS3 such as “Please avoid a dark portion” may be displayed in a superimposed manner on the correction image 161.

As described above, the change of the display mode in the correction image 161 can notify the user that a low-reliability region including a relatively large number of disturbances is included in the specific region or notify the user that the correction processing can be appropriately performed. The notification may be performed via voice instead of or in addition to an image displayed on the display.

Such a notification can prompt the user to operate the endoscope 12 while observing the correction image 161 so that a region less influenced by disturbances falls within the specific region 162. That is, the user can be prompted to operate the endoscope 12 so that a low-reliability region does not fall within the specific region as much as possible and a high-reliability region falls within the specific region as much as possible.

When it is notified that the correction processing can be appropriately performed and an operation of giving an instruction to perform the correction processing is input by a user operation, the correction processing in the correction mode is performed. The reliability for each pixel in the specific region may be determined using the reliability determination threshold value and/or the reliability pixel number determination threshold value, and if it is determined that the specific region 162 is less influenced by disturbances, the correction processing may be automatically executed without the input operation by the user.

Alternatively, the extension processor device 16 may calculate the reliability in the specific region as an internal process without displaying the correction image 161 on the display, determine the reliability for each pixel, and then perform the correction processing using the image signals in the specific region.

When the correction processing is completed in the correction mode, display control is performed to prompt the user to switch to the oxygen saturation mode. Alternatively, the mode may be automatically switched to the oxygen saturation mode without such display being performed.

The correction processing may be performed such that a weighting process is performed on the signal values of the B2 image signal, the G2 image signal, the R2 image signal, the B3 image signal, and/or the G3 image signal using the reliability calculated for each pixel in the specific region to reflect the reliability in the correction processing. In the correction processing, when the X-component value (the signal ratio ln(R2/G2)), the Y-component value (the signal ratio ln(B1/G2)), and the Z-component value (the signal ratio ln(B3/G3)) are to be calculated using the mean values (mean signal values) of the signal values of the B2 image signal, the G2 image signal, the R2 image signal, the B3 image signal, and/or the G3 image signal in the specific region, the X-component value, the Y-component value, and the Z-component value may be calculated using weighted averages obtained by weighting the mean signal values.

Control for displaying a biological index value in a region of interest as a display image will be described hereinafter. The extension processor device 16 has the region-of-interest setting unit 210, the region position information storage unit 240, the region index value calculation unit 250, the index value display table generation unit 260, the display image generation unit 270, and the extension display control unit 200. The extension processor device 16 may further include a region index value storage unit 280 and/or an index value link line generation unit 290 described below.

The region-of-interest setting unit 210 sets a region of interest in the endoscopic image displayed on the first user interface 15 or the second user interface 17. The region of interest is a target region of the endoscopic image for calculating a region index value. The region index value is a statistical value of a biological index value calculated based on image signals in the region of interest. The biological index value is a value indicating the state of the observation target as the photographic subject. Specifically, the biological index value is an oxygen saturation, a hemoglobin index, or a combination index. The biological index value and the region index value will be described below.

A method for setting a region of interest will be described hereinafter with reference to a specific example of an endoscopic image in which a region of interest is to be set. Specific example (1) is a case where the endoscopic image in which the region of interest is set is an oxygen saturation image. Specific example (2) is a case where the endoscopic image in which the region of interest is set is a white-light-equivalent image. Specific example (3) is a case where the endoscopic image in which the region of interest is set is a white-light image.

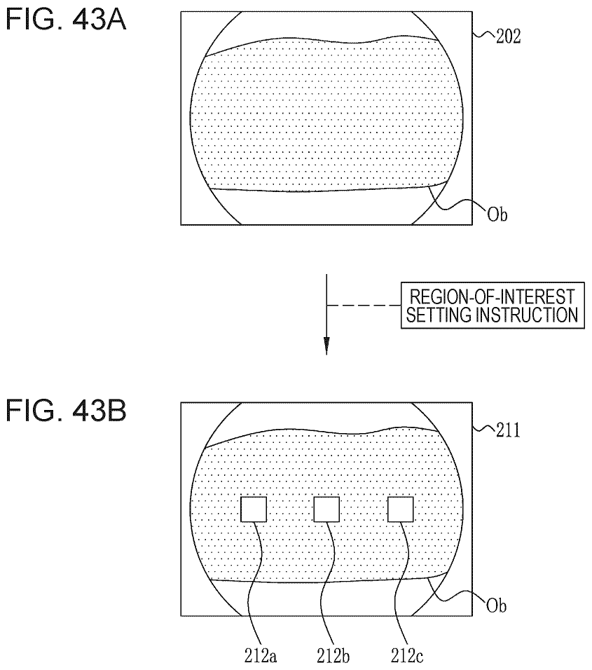

The specific example (1) will be described with reference to the drawings. For example, in the oxygen saturation mode, an oxygen saturation image 202 as illustrated in FIG. 43A is displayed on the display. In this case, when the user operates the region-of-interest setting switch 12d, a region-of-interest setting instruction is input to the extension processor device 16 via the central control unit 50 of the processor device 14. Upon receipt of the region-of-interest setting instruction, the region-of-interest setting unit 210 sets, for example, regions of interest 212a, 212b, and 212c on the endoscopic image (the oxygen saturation image 202), as illustrated in FIG. 43B. A plurality of regions of interest are set at different positions on the endoscopic image. In the example of the oxygen saturation image 202 illustrated in FIG. 43A, a large intestine observed from the serosa side using the endoscope 12, which is a laparoscope, is illustrated as a photographic subject Ob. In the subsequent drawings, the reference numeral of the photographic subject Ob is not illustrated to avoid complexity of the drawings. In the drawings, furthermore, the regions of interest or pieces of region position information described below are drawn to be arranged in a straight line for simplicity. However, it is preferable that the regions of interest to be set are not arranged in a straight line.

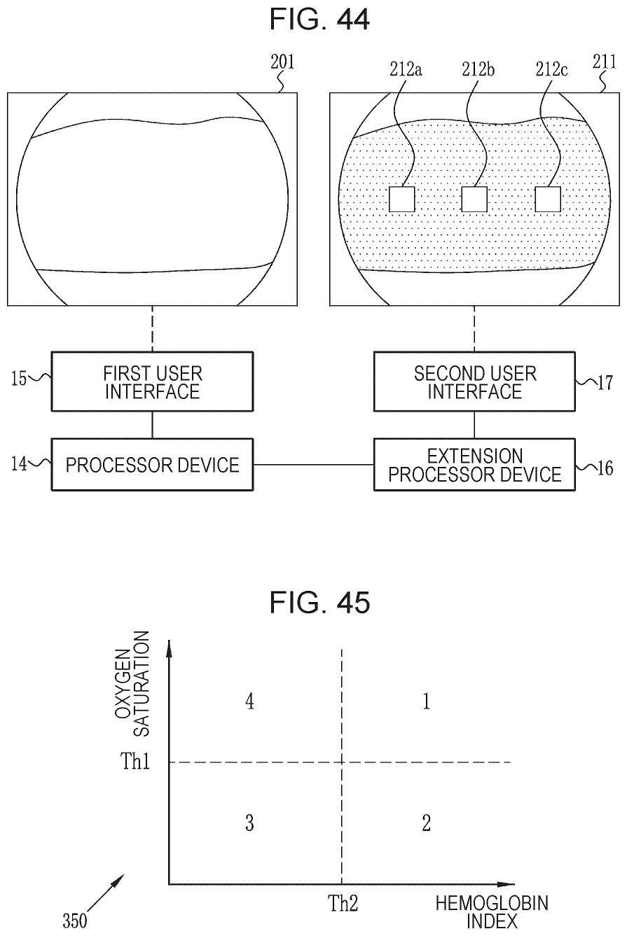

In response to a region-of-interest setting instruction being input to the extension processor device 16, the endoscopic image as illustrated in FIG. 43B in which the regions of interest 212a, 212b, and 212c are set (hereinafter referred to as a region-of-interest image 211) is displayed on the display as the second user interface 17, as illustrated in FIG. 44, under the display control of the extension display control unit 200. In FIG. 44, the region-of-interest image 211 obtained by setting regions of interest an oxygen saturation image is displayed on the display serving as the second user interface 17, and the white-light-equivalent image 201 is displayed on the display serving as the first user interface 15.

The region-of-interest setting unit 210 displays a region of interest at a preset position on an endoscopic image. It is preferable that the number and positions of regions of interest on the endoscopic image are set in advance. For example, in the example of the region-of-interest image illustrated in FIGS. 43B and 44, it is set in advance that “three” regions of interest are set. In addition, “set positions” of the three regions of interest 212a, 212b, and 212c are set in advance. The number of regions of interest can be set as any value that is a natural number of 2 or more. In addition, the positions of the regions of interest can be set as desired. The reason that the plurality of regions of interest are set at different positions is to calculate region index values in the regions of interest located at spatially separate positions. In other words, the spatially separate positions mean different positions in the biological tissue. The regions of interest are set at different positions on the biological tissue. As a result, the spatial change of the index values, that is, the index values in the regions of interest set at different positions in the biological tissue and the change of these index values, can be identified.

When the regions of interest are set on the endoscopic image, the positions of the regions of interest are stored in the region position information storage unit 240 as a plurality of pieces of region position information. That is, the positions of the plurality of regions of interest in the region-of-interest image are the pieces of region position information. The positions of the regions of interest are coordinate information of the regions of interest in the endoscopic image. The region position information storage unit 240 may be provided in the extension processor device 16 or may be a storage external to the extension processor device 16.

The operation of the region-of-interest setting switch 12d may be an operation of pressing the region-of-interest setting switch 12d included in the endoscope 12, a footswitch serving as a user interface, or the like, or may be a selection operation such as clicking or tapping of a region-of-interest setting switch serving as a graphical user interface (GUI) displayed on the display.

In a case where the region-of-interest image 211 as illustrated in FIGS. 43B and 44 in which the regions of interest 212a, 212b, and 212c are displayed is displayed on the display, in response to the user operating the region-of-interest setting switch 12d again, biological index values are calculated based on the image signals in the regions of interest 212a, 212b, and 212c. The calculated biological index values for the regions of interest are transmitted to the region index value calculation unit 250.

In the region-of-interest image 211 as illustrated in FIGS. 43B and 44, the oxygen saturation may be calculated substantially in real time based on the B1 image signal, the B2 image signal, the G2 image signal, the R2 image signal, and the like in the regions of interest, and the hemoglobin index or the combination index may be calculated as a biological index value.

For example, the hemoglobin index indicating the blood concentration of the photographic subject may be calculated based on the B1 image signal, the B2 image signal, the G2 image signal, the R2 image signal, and the like having blood concentration dependence. Alternatively, the combination index, which is a biological index value obtained by combining the oxygen saturation and the hemoglobin index, may be calculated.

The combination index is calculated using a combination index calculation table 350 as illustrated in FIG. 45. In the combination index calculation table 350, a threshold value is provided for each of the oxygen saturation and the hemoglobin index. Thus, the combination index, which is the value “1”, “2”, “3”, or “4”, can be determined in accordance with the levels of the oxygen saturation and the hemoglobin index.

In the combination index calculation table 350 illustrated in FIG. 45, when the oxygen saturation (vertical axis) is greater than or equal to an oxygen saturation threshold value Th1 and the hemoglobin index (horizontal axis) is greater than or equal to a hemoglobin index threshold value Th2, the combination index is “1”. The combination index is “2” when the oxygen saturation is less than the oxygen saturation threshold value Th1 and the hemoglobin index is greater than or equal to the hemoglobin index threshold value Th2. The combination index is “3” when the oxygen saturation is less than the oxygen saturation threshold value Th1 and the hemoglobin index is less than the hemoglobin index threshold value Th2. The combination index is “4” when the oxygen saturation is greater than or equal to the oxygen saturation threshold value Th1 and the hemoglobin index is less than the hemoglobin index threshold value Th2.

A pixel or a region having the combination index “1”, “2”, or “3” is of low oxygen saturation or congestion and has a risk of causing suture failure. In contrast, a pixel or a region having the combination index “4” is a region of high oxygen saturation and low hemoglobin index and without congestion, with a low risk of causing suture failure.

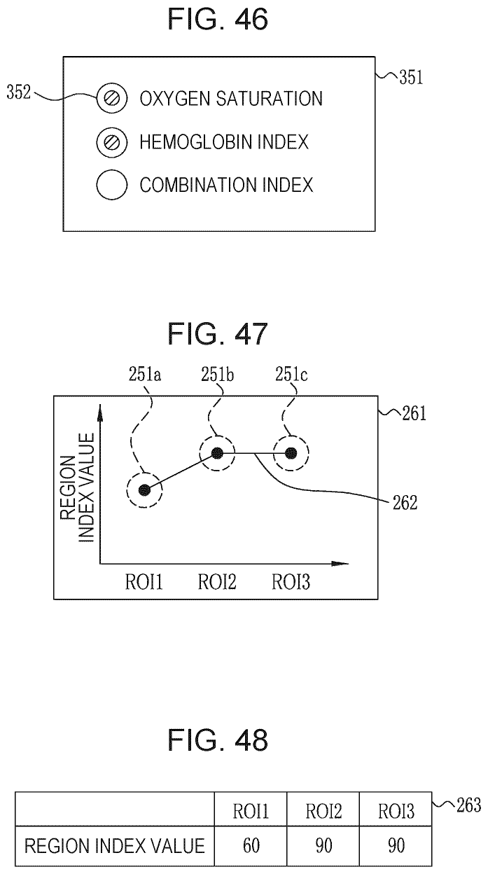

It is preferable that which biological index value to calculate can be changed by a user setting. For example, a biological index value selection screen 351 as illustrated in FIG. 46 may be displayed on the display, and a radio button 352, which is a GUI, may be operated to select a biological index value to be calculated. In the example illustrated in FIG. 46, the oxygen saturation and the hemoglobin index are selected as biological index values to be calculated.

When the oxygen saturation is to be calculated as a biological index value, the oxygen saturation is calculated by the oxygen saturation calculation unit 133. When the hemoglobin index or the combination index is to be calculated, the extension processor device 16 may be provided with a biological index value calculation unit (not illustrated), and the biological index value calculation unit may calculate these biological index values.

The region index value calculation unit 250 calculates a region index value as a statistical value of biological index values in a region of interest, based on the biological index values in the region of interest. The statistical value is a mean, a median, a mode, a maximum, a minimum, or the like. The region index value is calculated for each region of interest. In the example illustrated in FIG. 43B, respective region index values are calculated for the region of interest 212a, the region of interest 212b, and the region of interest 212c. The calculated region index values are transmitted to the index value display table generation unit 260. The region index values may be transmitted to the region index value storage unit 280 described below.

The index value display table generation unit 260 collects the plurality of region index values to generate an index value display table to be displayed in a display image. For example, the index value display table generation unit 260 generates an index value display table 261 as illustrated in FIG. 47 in which the plurality of region index values are represented in a graph format. The example of the index value display table 261 illustrated in FIG. 47 presents an example of an index value display table 261 with the vertical axis representing the region index value and the horizontal axis representing the region of interest. In the index value display table 261 illustrated in FIG. 47, the region of interest 212a is indicated by “ROI1”, the region of interest 212b is indicated by “ROI2”, and the region of interest 212c is indicated by “ROI3”, and a line sparkline 262 indicating corresponding region index values 251a, 251b, and 251c is displayed (see FIG. 43B). The “ROI” is an acronym for Region of Interest and means an “area of interest”.

In a case where an index value display table is generated in which the region index values are represented in a graph format, a sparkline may be displayed as a vertical bar graph. In the example of the index value display table 261 illustrated in FIG. 47, the region index value 212a of the region of interest 251a (ROI1) is calculated as “60”, the region index value 251b of the region of interest 212b (ROI2) is calculated as “90”, and the region index value 251c of the region of interest 212c (ROI3) is calculated as “90”. The calculated region index values 251a, 251b, and 251c may be displayed together with a sparkline 262. In the specific example illustrated in FIG. 47, it is assumed that the biological index value is calculated as the oxygen saturation.

The index value display table generation unit 260 may generate an index value display table 263 as illustrated in FIG. 48 in which the plurality of region index values are represented in a table format. FIG. 48 illustrates the index value display table 263 in which the region index values are represented in a table format when the region index value of the region of interest 212a is calculated as “60”, the region index value of the region of interest 212b is calculated as “90”, and the region index value of the region of interest 212c is calculated as “90”.

Whether the index value display table generation unit 260 generates an index value display table in a graph format or a table format, and whether the index value display table generation unit 260 generates a line sparkline or a vertical bar sparkline in an index value display table in a graph format may be set in advance or may be set by the user. In a case where these settings are to be performed by the user, a setting screen for an index value display table (not illustrated) may be displayed on the display, and the user may operate the GUI to perform the settings. The index value display table generated by the index value display table generation unit 260 is transmitted to the display image generation unit 270.

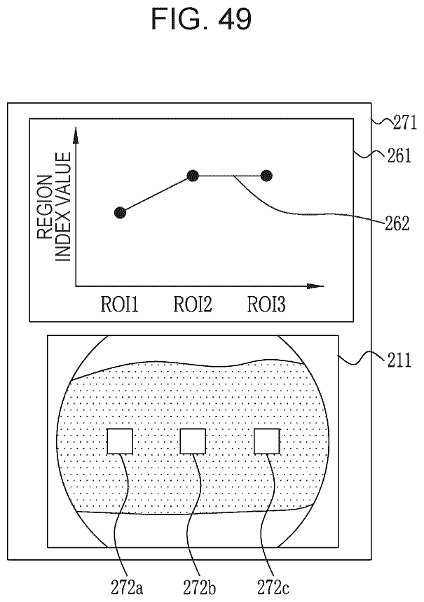

The display image generation unit 270 generates a display image for displaying the index value display table, the endoscopic image, and the region position information. For example, in generating a display image that displays an index value display table as illustrated in FIG. 47, the display image generation unit 270 generates a display image 271 as illustrated in FIG. 49. In the example of the display image 271 illustrated in FIG. 49, an endoscopic image (the region-of-interest image 211 obtained by setting the regions of interest in the oxygen saturation image 202, see FIG. 43B) and the index value display table 261 are displayed. In the display image 271, pieces of region position information 272a, 272b, and 272c, which are pieces of information on the positions of the target regions of interest for calculating the region index values, are further displayed in a superimposed manner on the endoscopic image.

The display image generation unit 270 reads region position information indicating the position of a region of interest in the endoscopic image and stored in the region position information storage unit 240, and displays the position of the target region of interest for calculating the region index value, as the region position information, in a superimposed manner on the endoscopic image to be displayed on the display image. In the example of the display image 271 illustrated in FIG. 49, the region position information 272a corresponds to the region of interest 212a, the region position information 272b corresponds to the region of interest 212b, and the region position information 272c corresponds to the region of interest 212c (see FIGS. 43B and 44).

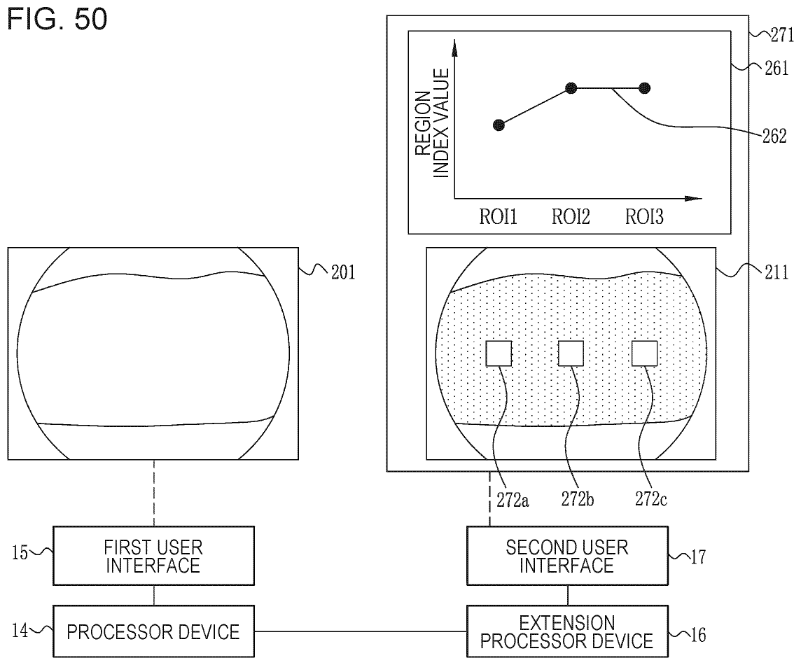

The generated display image is transmitted to the extension display control unit 200. The extension display control unit 200 performs signal processing for displaying the display image on the display to display the display image on the display serving as the second user interface 17. In a case where a region of interest is set in the oxygen saturation image 202, for example, as illustrated in FIG. 50, the white-light-equivalent image 201 is displayed on the display serving as the first user interface 15, and the display image 271 is displayed on the display serving as the second user interface 17.

As described above, the display of an index value display table that displays region index values related to regions of interest in a plurality of locations in an oxygen saturation image can notify the user of the spatial change of biological index values in the oxygen saturation image being observed by the user. Furthermore, as in the specific example (1), in the oxygen saturation mode, a white-light-equivalent image and a display image are displayed side by side. Thus, an image close to white light and region index values in a plurality of locations on an oxygen saturation image can be displayed to the user in an easy-to-compare manner. As a result, it is possible to support the user in determining a portion that is a possible appropriate incision site based on the biological index values or a portion inappropriate for incision based on the biological index values.

The specific example (2) will be described hereinafter. Unlike the specific example (1) in which regions of interest are set in an oxygen saturation image, in the specific example (2), in the oxygen saturation mode, a plurality of regions of interest are set on a white-light-equivalent image. In addition, when a display image is to be finally displayed, the display image generated by the display image generation unit 270 is transmitted to the display control unit 80 of the processor device 14, and is displayed on the display serving as the first user interface 15.

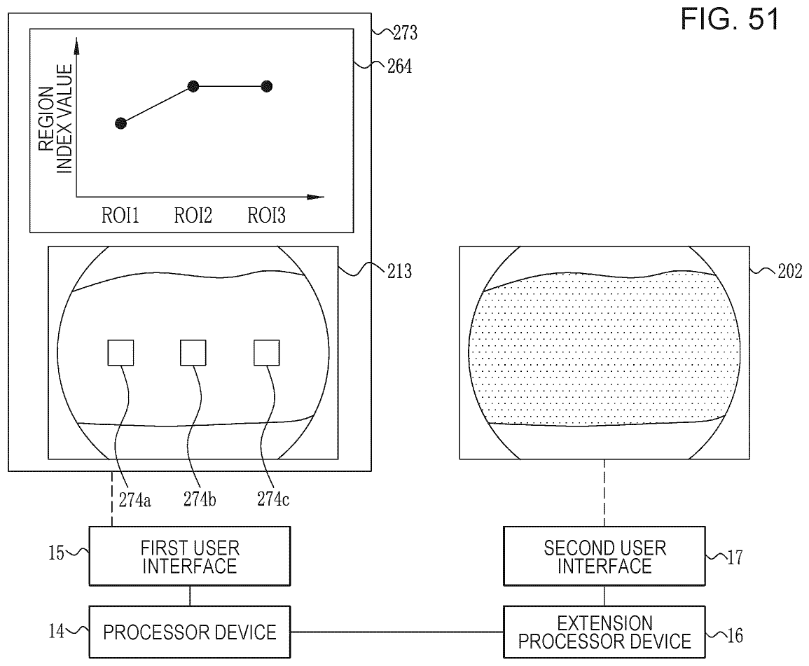

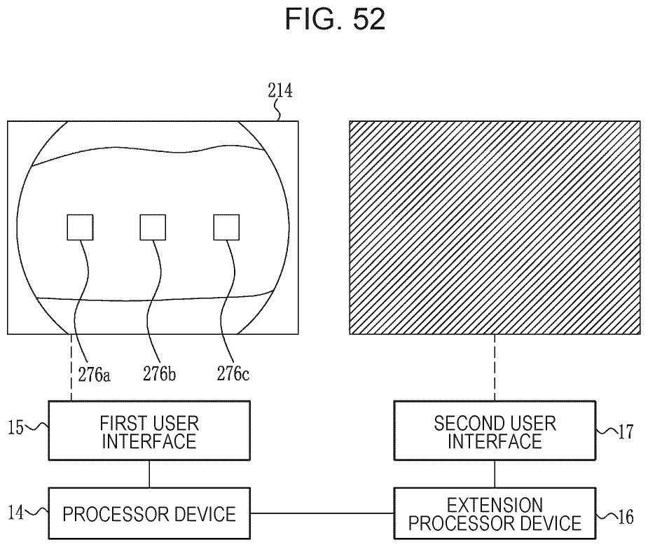

In the specific example (2), as illustrated in FIG. 51, a display image 273 is displayed on the display serving as the first user interface 15, and the oxygen saturation image 202 is displayed on the display serving as the second user interface 17. In the example of the display image 273 illustrated in FIG. 51, pieces of region position information 274a, 274b, and 274c indicating the positions of the set regions of interest are displayed in a superimposed manner on a white-light-equivalent image 213. An index value display table 264 that collectively displays the region index values for the regions of interest set in the white-light-equivalent image 213 is also displayed.

A specific process flow in the specific example (2) will be described. First, in the oxygen saturation mode, when a region-of-interest setting instruction is input to the extension processor device 16 by the user operating the region-of-interest setting switch 12d, a plurality of regions of interest are set on a white-light-equivalent image.

The process flow in the specific example (2) from the setting of the regions of interest on the white-light-equivalent image to the generation of a display image is similar to that in the specific example (1), and thus a detailed description thereof will be omitted. A brief description will be given hereinafter. The positions of the plurality of regions of interest set on the white-light-equivalent image are stored in the region position information storage unit 240 as pieces of region position information. When a region-of-interest image that displays the regions of interest on the white-light-equivalent image is displayed on the display, in response to the region-of-interest setting switch 12d being operated again, biological index values are calculated based on the image signals in the regions of interest. Subsequently, the region index value calculation unit 250 calculates region index values based on the biological index values for the regions of interest. Subsequently, the index value display table generation unit 260 uses the calculated respective region index values for the regions of interest to generate the index value display table 264 in which the plurality of region index values are collected. Finally, the display image generation unit 270 generates a display image for displaying the index value display table 264 and the white-light-equivalent image 213 on which the pieces of region position information 274a, 274b, and 274c are displayed in a superimposed manner.

As in the specific example (2) described above, in the oxygen saturation mode, a display image and an oxygen saturation image are displayed side by side. Thus, the oxygen saturation image and region index values in a plurality of locations on a white-light-equivalent image can be displayed to the user in an easy-to-compare manner.

The specific example (3) will be described hereinafter. In the specific example (3), unlike the specific example (1) and the specific example (2), in the normal mode, a plurality of regions of interest are set in a white-light image. In addition, a mode switching operation is performed using the mode switching switch 12c instead of the region-of-interest setting switch 12d to input a region-of-interest setting instruction.

A specific process flow in the specific example (3) will be described. First, in the normal mode, the user operates the mode switching switch 12c to set a plurality of regions of interest in a white-light image. In this case, in response to the operation of the mode switching switch 12c as a trigger, a region-of-interest setting instruction is input to the extension processor device 16. The region-of-interest setting unit 210 transmits a command signal to the display control unit 80 of the processor device 14 to cause the display serving as the first user interface 15 to display a region-of-interest image in which the endoscopic image is the white-light image.

In this case, as illustrated in FIG. 52, a region-of-interest image 214 in which the endoscopic image is the white-light image is displayed on the display serving as the first user interface 15, and nothing is displayed on the display serving as the second user interface 17 (no display is indicated by hatching). In the example illustrated in FIG. 52, nothing is displayed on the display serving as the second user interface 17. However, the white-light-equivalent image 201 or the oxygen saturation image 202 may be displayed on the display serving as the second user interface 17. In this case, furthermore, regions of interest are set on the white-light image substantially at the same time as the switching of the mode.

The process flow in the specific example (3) from the setting of the regions of interest on the white-light image to the generation of a display image is similar to that in the specific example (1), and thus a detailed description thereof will be omitted. A brief description will be given hereinafter. The positions of the plurality of regions of interest set on the white-light image are stored in the region position information storage unit 240 as pieces of region position information. When the region-of-interest image 211 displaying the regions of interest on the white-light image is displayed on the display, in response to the region-of-interest setting switch 12d being operated, biological index values are calculated based on the image signals in the regions of interest. Subsequently, the region index value calculation unit 250 calculates region index values based on the biological index values for the regions of interest. Subsequently, the index value display table generation unit 260 uses the calculated respective region index values for the regions of interest to generate an index value display table in which the plurality of region index values are collected. Finally, the display image generation unit 270 generates a display image for displaying the index value display table and the white-light image on which the pieces of region position information are displayed in a superimposed manner.

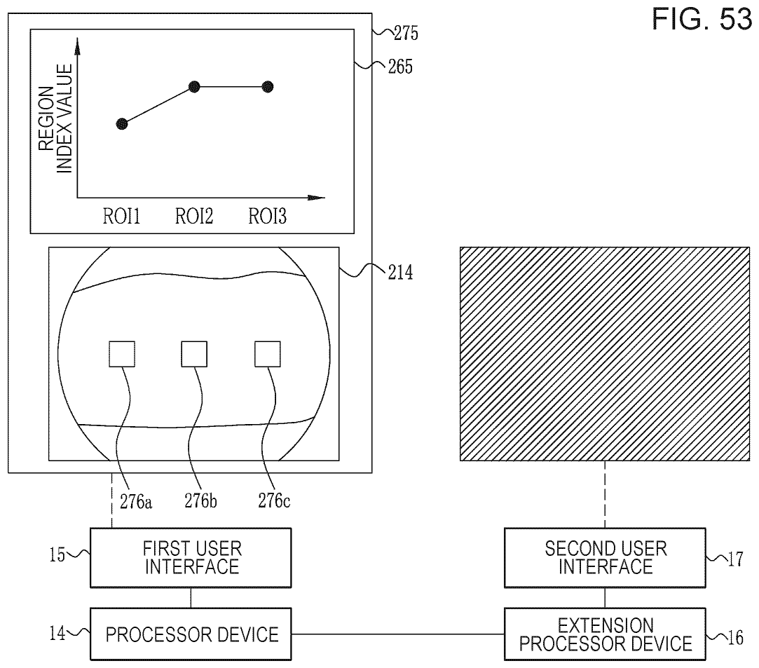

In the specific example (3), as illustrated in FIG. 53, a display image 275 is displayed on the display serving as the first user interface 15, and nothing is displayed on the display serving as the second user interface 17. In the example of the display image 275 illustrated in FIG. 53, pieces of region position information 276a, 276b, and 276c indicating the positions of the set regions of interest are displayed in a superimposed manner on the region-of-interest image 214 in which the endoscopic image is the white-light image. An index value display table 265 that collectively displays the region index values for the regions of interest set in the region-of-interest image 214 is also displayed. In the example illustrated in FIG. 53, nothing is displayed on the display serving as the second user interface 17. However, the white-light-equivalent image 201 or the oxygen saturation image 202 may be displayed on the display serving as the second user interface 17.

As indicated in the specific examples (1), (2), and (3) described above, an image that displays regions of interest, that is, an endoscopic image serving as the background of a region-of-interest image, may be an oxygen saturation image (the specific example (1)), a white-light-equivalent image (the specific example (2)), or a white-light image (the specific example (3)). Alternatively, the endoscopic image may be a special-light image that is captured using reflected light obtained by irradiating the photographic subject with special light other than the first illumination light, the second illumination light, and the third illumination light.

In a case where regions of interest are set in a white-light image or a special-light image and a display image in which an index value display table is displayed is to be generated, an endoscopic image that displays pieces of region position information (i.e., the white-light image or the special-light image serving as the background of the region-of-interest image) may be an image generated based on image signals acquired in real time or may be a still image generated based on image signals acquired immediately before the switching of the mode.

When the white-light image or the special-light image serving as the background of the region-of-interest image is an image generated based on the image signals acquired in real time, white light or special light is added as the illumination light to be emitted in the oxygen saturation mode in addition to the first illumination light and the second illumination light. Accordingly, the light emission pattern is changed such that an illumination period during which the white light or the special light is emitted is provided in the light emission pattern (see FIG. 15) in the oxygen saturation mode.

The specific examples (1), (2), and (3) present an example in which the biological index values and the region index values are calculated by the user operating the region-of-interest setting switch 12d while the region-of-interest image is displayed on the display. However, in response to a region-of-interest setting instruction being input, a display image may be displayed without the region-of-interest image being displayed on the display. For example, in the oxygen saturation mode, when, as illustrated in FIG. 7, the white-light-equivalent image 201 and the oxygen saturation image 202 are being displayed, in response to the operation of the region-of-interest setting switch 12d, the display image may be displayed in the display mode as illustrated in FIG. 50 or 51 (without using the display mode as illustrated in FIG. 44).

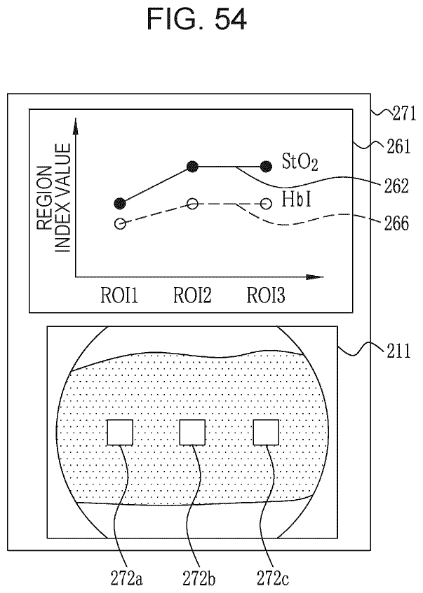

Further, when a plurality of types of biological index values are to be calculated for a plurality of regions of interest, region index values related to the plurality of types of biological index values may be calculated to generate an index value display table. For example, in a case where the oxygen saturation and the hemoglobin index are calculated as the biological index values, as illustrated in FIG. 54, an index value display table may be generated that displays a line sparkline 262 (indicated by a solid line) indicating the biological index value as the oxygen saturation (StO₂) and a line sparkline 266 (indicated by a broken line) indicating the biological index value as the hemoglobin index (HbI).

As described above, the display of an index value display table that displays region index values related to regions of interest in a plurality of locations can notify the user of the spatial change of the real number values of the biological index values in the currently observed endoscopic image. Such an index value display table that collectively displays the real number values can present more reliable information to the user in an easy-to-compare manner as compared with a case where the change of the biological index values is displayed only with a color tone.

In surgery for cancer or the like, when an organ such as a large intestine is partially resected, if the organ is sutured while a portion with poor blood flow is left in a biological tissue, the sutured portion is insufficiently healed, and the possibility of suture failure increases. As in the configuration described above, the display of an index value display table that enables easy and accurate distinction between a portion having a high biological index value and a portion having a low biological index value is useful for preventing suture failure.

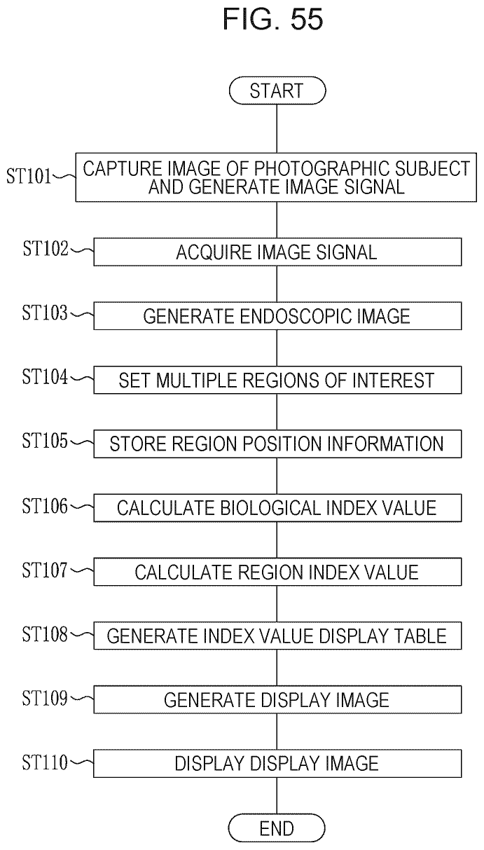

A series of process flow steps for performing display control of a display image on which an endoscopic image and an index value display table are displayed according to the first embodiment will be described with reference to a flowchart illustrated in FIG. 55. First, the endoscope 12 captures reflected light from a photographic subject to generate an image signal (ST101). Next, the image signal acquisition unit 60 of the processor device 14 acquires the image signal generated by the endoscope 12 (ST102). Next, the endoscopic image generation unit 70 of the processor device 14 and/or the oxygen saturation image generation unit 130 of the extension processor device 16 generates an endoscopic image based on the image signal (ST103). Next, the region-of-interest setting unit 210 sets a plurality of regions of interest in the endoscopic image (ST104). Next, the positions of the plurality of regions of interest in the endoscopic image are stored in the region position information storage unit 240 as a plurality of pieces of region position information (ST105).

Next, a biological index value is calculated based on the image signal in each region of interest (ST106). Next, the region index value calculation unit 250 calculates a region index value for each region of interest, based on the biological index value in the corresponding region of interest (ST107). Next, the index value display table generation unit 260 generates an index value display table that collectively displays the plurality of region index values (ST108). Next, the display image generation unit 270 generates a display image for displaying the index value display table and the endoscopic image on which the plurality of pieces of region position information are displayed in a superimposed manner (ST109). Finally, the extension display control unit 200 performs control to display the display image (ST110). As a result, the display image is displayed on the display serving as the user interface.

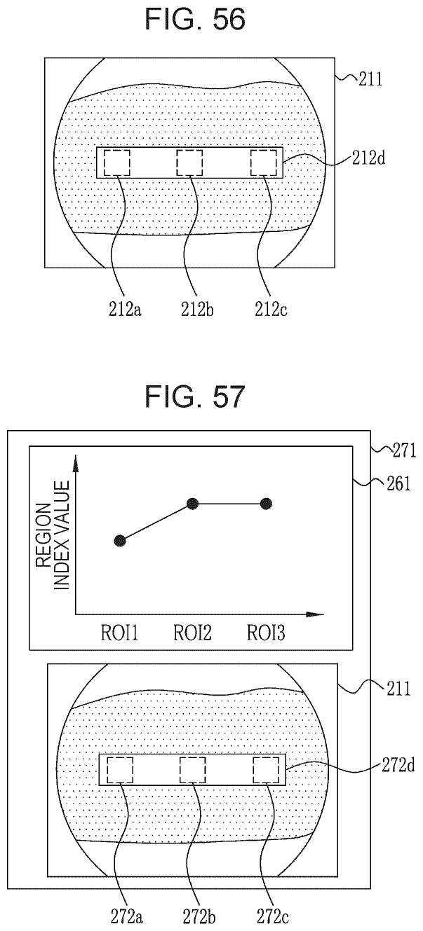

In the region-of-interest image displayed on the display, the plurality of regions of interest may be collectively displayed as one display region of interest. For example, instead of the region-of-interest image 211 as illustrated in FIG. 43B in which the plurality of regions of interest 212a, 212b, and 212c are displayed, a display region of interest 212d as illustrated in FIG. 56 may be displayed. The display region of interest 212d illustrated in FIG. 56 includes the plurality of regions of interest 212a, 212b, and 212c, as indicated by broken lines indicating that the plurality of regions of interest 212a, 212b, and 212c are not actually displayed.

Also in the case of displaying the region-of-interest image 211 as illustrated in FIG. 56, the region index values for the plurality of regions of interest 212a, 212b, and 212c are calculated. As a display image, for example, as illustrated in FIG. 57, the region-of-interest image 211 displays display-region position information 272d that includes the pieces of region position information 272a, 272b, and 272c, which are indicated by broken lines and are not actually displayed. The collective display of a plurality of regions of interest can reduce the apparent amount of information included in the display image and improve the visibility of the display image. In the example of the display image 271 illustrated in FIG. 57, the reference numeral of the line sparkline 262 and the leader line are omitted. In the subsequent drawings, the reference numeral of the line sparkline 262 and the leader line are also omitted for easy understanding of the drawings.

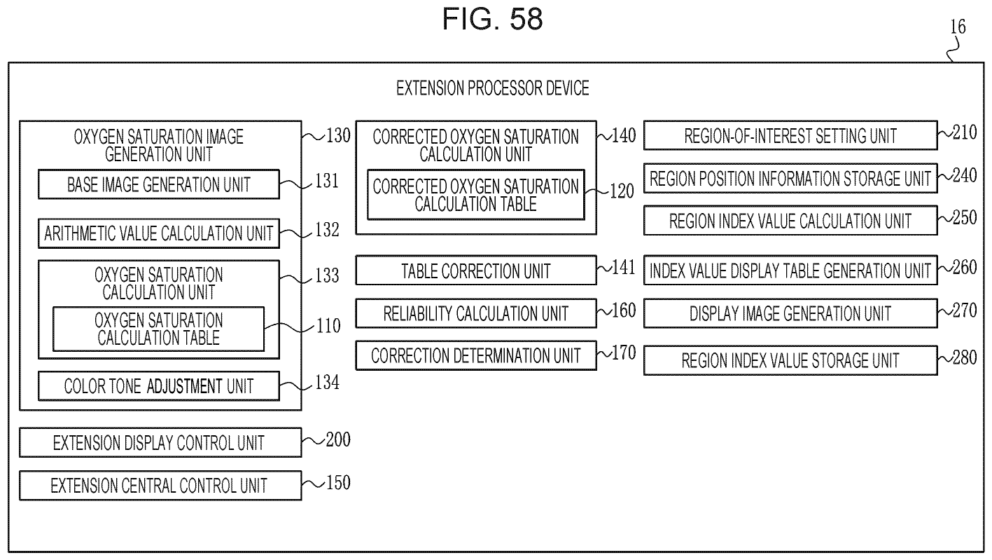

As illustrated in FIG. 58, the extension processor device 16 may further be provided with the region index value storage unit 280. The region index value storage unit 280 may associate region position information with a region index value calculated in a region of interest indicated by the region position information to store a specific region index value in which the region position information and the region index value are associated with each other, and the specific region index value may be held and displayed in an index value display table on a display image.

In a case where the specific region index value is held and displayed in the index value display table, the region index value is calculated only once after a biological index value is calculated. The region index value calculation unit 250 calculates a region index value for each region of interest, based on the biological index value in the corresponding region of interest (see ST106 and ST107 in FIG. 55). The region index value calculated at this time is stored in the region index value storage unit 280 as a specific region index value in association with the region position information. The region index value storage unit 280 may be included in the extension processor device 16 or may be a storage external to the extension processor device 16.

The index value display table generation unit 260 generates an index value display table that collectively displays specific region index values calculated in respective regions of interest. The display image generation unit 270 generates a display image that displays the index value display table in which the specific region index values are displayed. In this case, the specific region index values in the display image displayed on the user interface by the extension display control unit 200 are held and displayed as fixed values.

The display of a plurality of specific region index values as fixed values allows the user to visually compare the plurality of region index values while carefully observing the index value display table when the vitals of the subject are stable and the change of the biological index values is small.

Further, the region index values displayed in the index value display table on the display image may be updated. The region index value calculation unit 250 calculates a region index value for each region of interest, based on the biological index value in the corresponding region of interest (see ST106 and ST107 in FIG. 55). The index value display table generation unit 260 generates an index value display table that collectively displays the specific region index values calculated in the respective regions of interest (see ST108 in FIG. 55). Each time a region index value is calculated, the index value display table generation unit 260 generates an index value display table in which the newly calculated latest region index value is reflected, to update each of the plurality of region index values displayed in the index value display table.

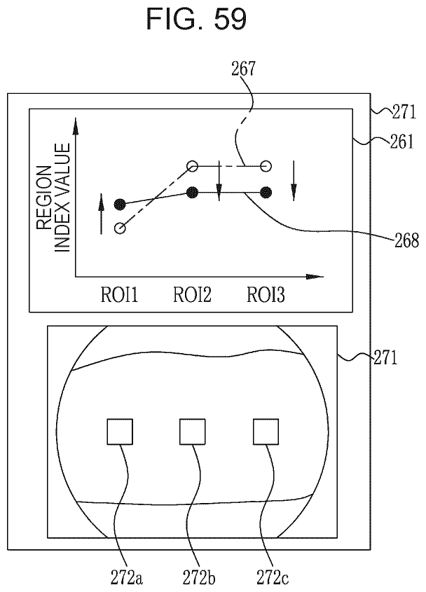

FIG. 59 illustrates a specific example of the display image 271 for updating the region index values displayed in the index value display table 261. When a region index value is newly calculated, the index value display table generation unit 260 updates a line sparkline 267 (indicated by a dash-dotted line in FIG. 59) generated at the chronologically previous time point to a line sparkline 268 (indicated by a solid line in FIG. 59) generated at the chronologically next time point.

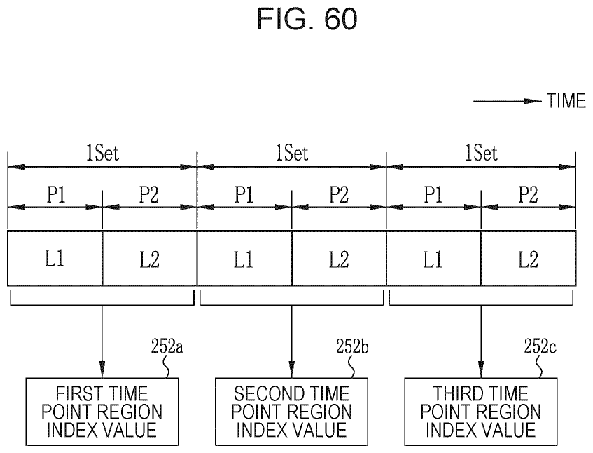

The update of a region index value will be described in detail. A specific example will be described in which a region index value is chronologically calculated in the region of interest indicated by the region position information 272a. For example, when illumination light is emitted in the oxygen-saturation-mode light emission pattern, as illustrated in FIG. 60, a first time point region index value 252a, a second time point region index value 252b, and a third time point region index value 252c are each calculated chronologically in each frame set (“1Set” in FIG. 60) including an illumination period in which the first illumination light L1 is emitted once and an illumination period in which the second illumination light L2 is emitted once. The index value display table generation unit 260 generates an index value display table such that the index value display table is updated each time a new region index value, such as the first time point region index value 252a, the second time point region index value 252b, or the third time point region index value 252c, is calculated in the region of interest indicated by the region position information 272a.

The update and display of the index value display table can present, to the user, region index values that are updated substantially in real time. The user can check the real number values of the biological index values in a plurality of locations substantially in real time in a scene with a large change in blood flow, such as during treatment or immediately after treatment.

In FIG. 60, a region index value is calculated for each frame set. The frame set for which a region index value is calculated can be set as desired. For example, a region index value can be set to be calculated for every other frame set such that a region index value is calculated for a certain frame set, no region index value is calculated for the next frame set, and a region index value is calculated for the next frame set. It is preferable that whether to hold or update the region index value to be displayed on the display image can be set by a user operation.

The region-of-interest setting unit 210 sets regions of interest at a plurality of preset different positions on the endoscopic image. Alternatively, a region of interest that has been set may be stored as a lock-on area, and the lock-on area may be displayed so as to follow the movement of the endoscope 12. Further, to update the region index value, a new region index value may be calculated in the region of interest stored as the lock-on area.

When the lock-on area is stored, the region of interest that has been set and the region position information of the region of interest are associated with each other and are thus stored as lock-on area position information indicating the position of the lock-on area. The lock-on area position information is coordinate information of the lock-on area on the endoscopic image. For example, the region-of-interest setting unit 210 may be provided with a region position association unit (not illustrated), and the region position association unit may associate the region of interest that has been set and the region position information of the region of interest with each other.

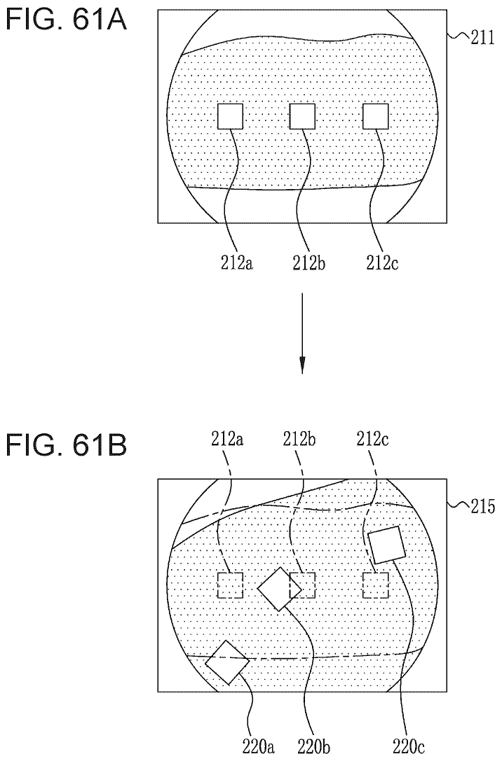

In a scene in which the range that the angle of view of the endoscope 12 covers is changed, such as when the endoscope 12 is moved from the current observation position or when the observation magnification of the endoscope 12 is changed, the range within which an image is to be captured may be significantly shifted from a position including a region of interest that has initially been set by the region-of-interest setting unit 210. For example, in the region-of-interest image 211 as illustrated in FIG. 61A, the region index values are calculated based on the image signals acquired in the regions of interest 212a, 212b, and 212c. It is assumed that the endoscope 12 is moved and further the observation magnification of the endoscope 12 is increased to change the endoscopic image to be displayed on the display from the region-of-interest image 211 as illustrated in FIG. 61A to a region-of-interest image 215 as illustrated in FIG. 61B.

In this case, the pieces of region position information of the regions of interest 212a, 212b, and 212c illustrated in FIG. 61A are stored as the lock-on area position information. In the region-of-interest image 215 as illustrated in FIG. 61B, the regions of interest move to follow lock-on areas 220a, 220b, and 220c (indicated by solid lines) from the regions 212a, 212b, and 212c (indicated by dash-dotted lines) illustrated in FIG. 61B. In this case, the region index value calculation unit 250 calculates the region index values on the basis of the biological index values in the lock-on areas 220a, 220b, and 220c.

It is indicated that the region of interest 212a illustrated in FIG. 61A moves to the lock-on area 220a illustrated in FIG. 61B, the region of interest 212b illustrated in FIG. 61A moves to the lock-on area 220b illustrated in FIG. 61B, and the region of interest 212c illustrated in FIG. 61A moves to the lock-on area 220c illustrated in FIG. 61B. The plurality of regions of interest 212a, 212b, and 212c are stored in the region position information storage unit 240 as lock-on area position information indicating the positions of the plurality of lock-on areas 220a, 220b, and 220c, respectively.

The amount of movement from the regions of interest 212a, 212b, and 212c illustrated in FIG. 61A to the lock-on areas 220a, 220b, and 220c illustrated in FIG. 61B is calculated based on the amount of movement, the amount of rotation, the rate of change of the observation magnification, and the like of the endoscope 12. In a case where the lock-on area position information is not stored, the region index values are calculated based on, also in FIG. 61B, the biological index values in the regions of interest 212a, 212b, and 212c. Accordingly, in a case where the endoscope 12 is to be moved largely, a region of interest that has been set is stored as a lock-on area. Thus, the region index value can be calculated in accordance with the movement of the endoscope 12.

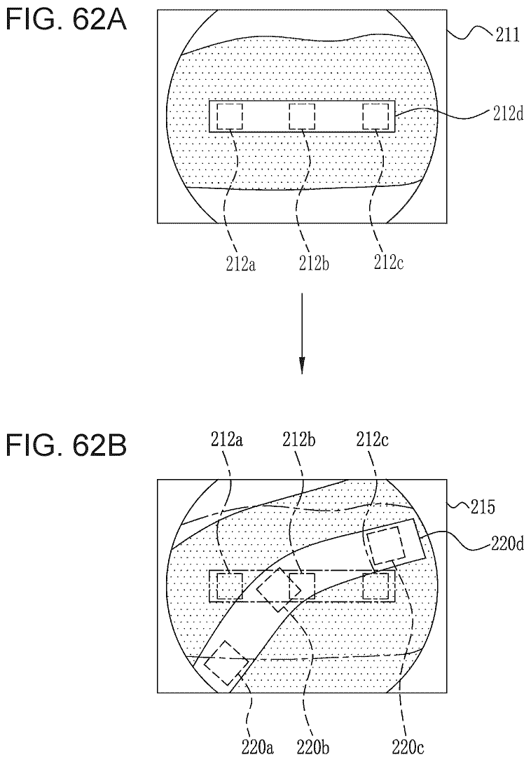

In the display image, the endoscopic image to be displayed on the display image is changed from the region-of-interest image 211 illustrated in FIG. 61A to the region-of-interest image 215 as illustrated in FIG. 61B in which the lock-on areas 220a, 220b, and 220c are displayed, in accordance with the operation of the endoscope 12. In a case where, as illustrated in FIG. 62A, the endoscopic image to be displayed on the display image displays the display region of interest 212d including the plurality of regions of interest 212a, 212b, and 212c, a display lock-on area 220d including the plurality of lock-on areas 220a, 220b, and 220c as illustrated in FIG. 62B is displayed in accordance with the operation of the endoscope 12.

In the observation of an endoscopic image in real time during examination, surgery, or the like, in some cases, the endoscope 12 may be moved from a position at which the observation target is currently being observed, and the observation target may be desired to be observed at a different position. Such cases include, for example, a case where the user searches for a position suitable for incision on the observation target. In a case where the observation magnification of the endoscope 12 is to be changed, the endoscopic image to be displayed on the display is switched from a long-range image to a close-range image or from a close-range image to a long-range image. The long-range image is an endoscopic image of the target observed at low magnification, which is suitable for wide-range observation. The close-range image is an endoscopic image of the target observed at high magnification, which is suitable for observation of a fine structure. As described above, the movement of the endoscope 12 or the change of the observation magnification of the endoscope 12 may cause the position of an initially set region of interest to be shifted from the position at which the region index value is actually to be calculated. Accordingly, an initially set region of interest is set as a lock-on area. Thus, even when the endoscope 12 is moved, the region index value for the initially set region of interest can be calculated.

In a case where a region of interest that has been set is stored as a lock-on area, it is preferable that a lock-on area setting switch (not illustrated) is operated to input a lock-on area storage instruction and lock-on area position information is stored.

The region index value (lock-on area index value) calculated based on the image signal in the lock-on area may be associated with the lock-on area position information, and accordingly, specific lock-on area index value in which the lock-on area position information and the lock-on area index value may be associated with each other is stored. The specific lock-on area index value is stored in the region index value storage unit 280. Further, the specific lock-on area index value is stored for each of a plurality of lock-on areas.

The specific lock-on area index value may be stored only once, or may be updated each time the lock-on area index value is calculated. Alternatively, each time the region index value for the lock-on area is calculated, the region index value may be associated with time-series data indicating a time series, and each specific lock-on area index value may be stored as information that can identify when the corresponding region index value is calculated.

The specific lock-on area index value is stored. Thus, the lock-on area index value (in this case, the specific lock-on area index value) to be displayed in the index value display table on the display image can be displayed such that the lock-on area index value calculated once is held. Further, each time the lock-on area index value is calculated, the lock-on area index value (in this case, the specific lock-on area index value) displayed in the index value display table on the display image can be updated.

In the observation of an endoscopic image in real time during examination, surgery, or the like, in some cases, the lock-on area is not included in the currently observed endoscopic image due to the movement of the endoscope 12 or an operation such as changing the observation magnification. As described above, when the lock-on area is at a position out of the field of view position, which is a position not included in the currently observed endoscopic image, it is preferable that the lock-on area index value in the lock-on area located at the position out of the field of view is continuously displayed in the index value display table on the display image.

In this case, the index value display table generation unit 260 sets the specific lock-on area index value stored immediately before the lock-on area is located at the position out of the field of view as an out-of-field-of-view lock-on area index value, and generates an index value display table that displays the out-of-field-of-view lock-on area index value.

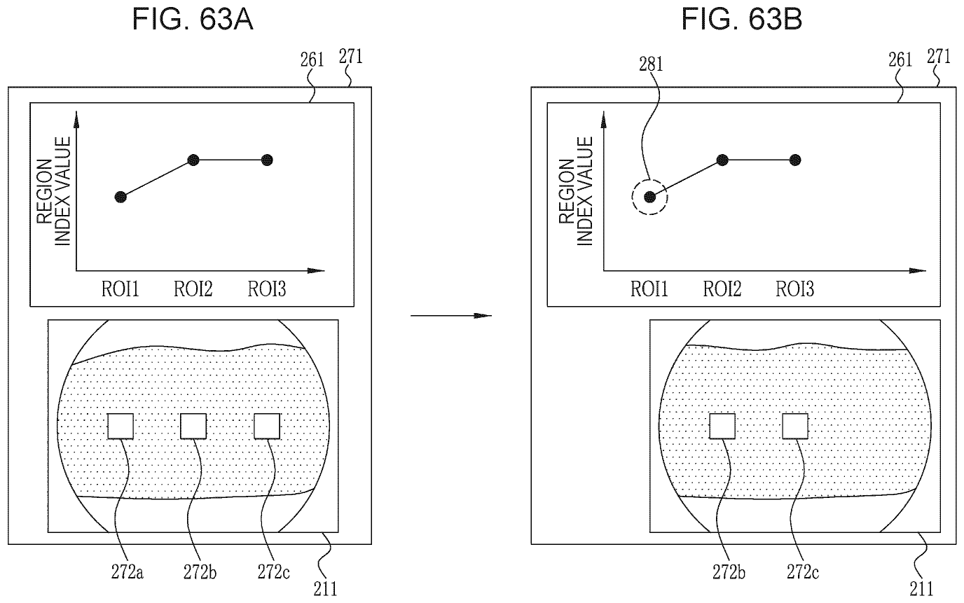

A specific example of the index value display table that displays the out-of-field-of-view lock-on area index value will be described with reference to FIGS. 63A and 63B. FIG. 63A illustrates the display image 271 before the endoscope 12 is moved. In FIG. 63A, the index value display table 261 is displayed in which region index values calculated in three lock-on areas depicted as the pieces of region position information 272a, 272b, and 272c are displayed. FIG. 63B illustrates the display image 271 after the endoscope 12 is moved.

In FIG. 63B, due to the movement of the endoscope 12, the position of the lock-on area depicted as the region position information 272a is at position out of the field of view. In FIG. 63B, accordingly, the two lock-on areas depicted as the pieces of region position information 272b and 272c are displayed. In this case, the region index value of the lock-on area depicted as the region position information 272a is stored as an out-of-field-of-view lock-on area index value. In this case, furthermore, the index value display table generation unit 260 generates the index value display table 261 in which an out-of-field-of-view lock-on area index value 281 is displayed. The extension display control unit 200 performs control to display the display image generated by the display image generation unit 270 for displaying the index value display table 261 in which the out-of-field-of-view lock-on area index value 281 is displayed, thereby displaying the display image as illustrated in FIG. 63B.

To display the out-of-field-of-view lock-on area index value, as illustrated in FIG. 63B, it is preferable to display a portion of the index value display table 261 in which the out-of-field-of-view lock-on area index value is displayed such that the portion of the index value display table 261 is shifted from the region-of-interest image 211 displayed on the display image, whereby the user can easily understand that the out-of-field-of-view lock-on area index value is displayed in the index value display table 261. This does not apply to a case where the display size of the index value display table 261 is changed, which will be described below.

As described above, the out-of-field-of-view lock-on area index value is displayed on the display image. Thus, the region of interest at the position out of the field of view can be presented to the user. The region index value outside the currently observed endoscopic image is displayed. Thus, information in a wide range of the photographic subject can be presented to the user. As a result, the user can search for a region suitable for incision while referring to information outside the currently observed endoscopic image.

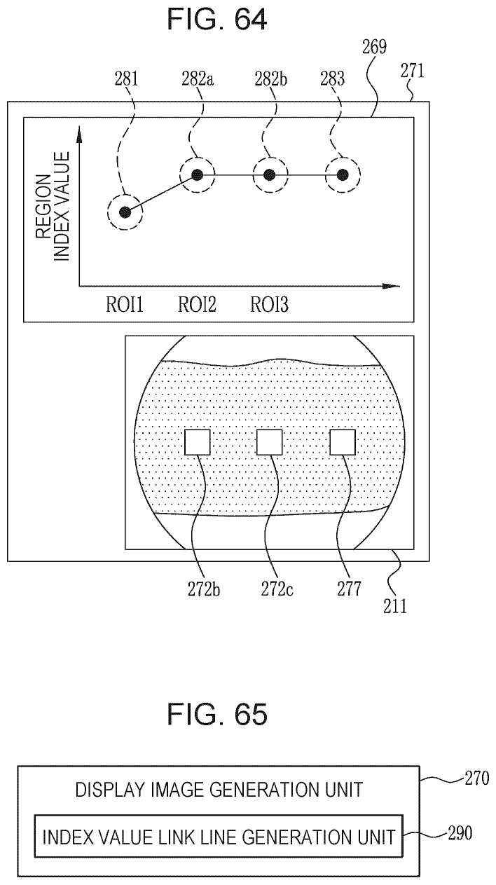

When the out-of-field-of-view lock-on area index value as illustrated in FIG. 63B is being displayed on the display image, it is preferable that a region of interest can be further added on the endoscopic image. An example of addition of a further region of interest will be described hereinafter with reference to FIG. 64.

In response to a region-of-interest setting instruction being input by a user operation when the out-of-field-of-view lock-on area index value as illustrated in FIG. 63B is being displayed on the display image, the region-of-interest setting unit 210 sets an additional region of interest 277 as illustrated in FIG. 64. The additional region of interest 277 in the example of the display image illustrated in FIG. 64 is, to be precise, region position information of the additional region of interest 277 to be displayed in a superimposed manner on the display image. The additional region of interest 277 may be a lock-on area.

When the additional region of interest 277 is set in response to the input of the region-of-interest setting instruction, the region position information of the additional region of interest 277 is stored in the region position information storage unit 240. The region index value calculation unit 250 calculates an additional region index value as a statistical value of biological index values calculated based on image signals in the additional region of interest 277. The additional region index value is a region index value that is a statistical value of biological index values calculated based on image signals in the additional region of interest 277.

The index value display table generation unit 260 generates an extension index value display table 269. The extension index value display table 269 is an index value display table that collectively displays the out-of-field-of-view lock-on area index value 281, region index values 282a and 282b of the two lock-on areas depicted as the pieces of region position information 272b and 272c, and an additional region index value 283. The extension display control unit 200 performs control to display the display image generated by the display image generation unit 270 for displaying the extension index value display table 269, thereby displaying a display image 271 as illustrated in FIG. 64. The extension index value display table is an index value display table that displays an additional region index value and is a form of the “index value display table”. In the extension index value display table, an additional region index value that has been calculated may be held and displayed, or may be updated and displayed each time an additional region index value is calculated.

In the example illustrated in FIG. 64, one additional region of interest 277 is newly set. Alternatively, a plurality of additional regions of interest may be set, a region index value may be calculated for each additional region of interest, and the plurality of region index values may be additionally displayed in the index value display table.

As described above, an additional region of interest is set, and an additional region index value is displayed. Thus, information in a wide range of the photographic subject can be presented to the user. In addition, the additional region index value is displayed in addition to the out-of-field-of-view lock-on area index value calculated chronologically before the additional region of interest is set. Thus, information in a wider range of the photographic subject can be presented to the user.

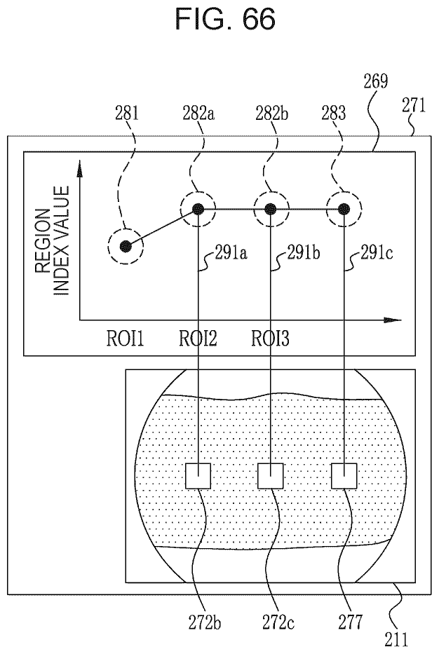

When the display image 271 as illustrated in FIG. 64 is displayed, it is preferable to display an index value link line connecting the pieces of region position information displayed in the display image and the region index values corresponding to the pieces of region position information. In this case, as illustrated in FIG. 65, the display image generation unit 270 is provided with an index value link line generation unit 290. An example of display of the index value link line will be described hereinafter with reference to FIG. 66.

In the example of a display image illustrated in FIG. 66, index value link lines 291a, 291b, and 291c are displayed in the display image 271 illustrated in the example in FIG. 64. The index value link line 291a is depicted to connect the region index value 282a and the region position information 272b. The index value link line generation unit 290 reads the association between the region index value 282a (lock-on area index value) and the region position information 272b (lock-on area position information), which is stored in the region index value storage unit 280, to generate the index value link line 291a connecting them, and the extension display control unit 200 performs control to perform display control.

The index value link line 291b is an index value link line connecting the region index value 282b and the region position information 272c. The index value link line 291c is an index value link line connecting the additional region index value 283 and the additional region of interest 277 depicted as the region position information. Like the index value link line 291a, the index value link line 291b and the index value link line 291c are generated by the index value link line generation unit 290, based on the association stored in the region index value storage unit 280.

As described above, an index value link line connecting a region index value displayed in the index value display table and region position information displayed in a superimposed manner on the endoscopic image is displayed. Thus, the correspondence relationship between the region index value and the region position information can be displayed in a manner easily understandable to the user. In particular, in a case where a larger number of region index values are to be displayed in the index value display table, the visibility of information for assisting in identifying a region suitable for incision can be improved.

Further, as in the example of the display image illustrated in FIG. 66, no index value link line is depicted for the region index value 281 that is the out-of-field-of-view lock-on area index value. The reason is to allow the user to easily understand that the lock-on area corresponding to the region index value 281 is at a position out of the field of view.

The extension display control unit 200 may change the display size of the index value display table displayed on the display image. The “change of the display size” includes a change to increase or decrease the display size with the aspect ratio of the index value display table maintained, and a change to increase or decrease the display size without the aspect ratio of the index value display table maintained. The change to increase or decrease the display size without the aspect ratio of the index value display table maintained includes a change to increase or decrease a distance between adjacent region index values displayed in the index value display table.

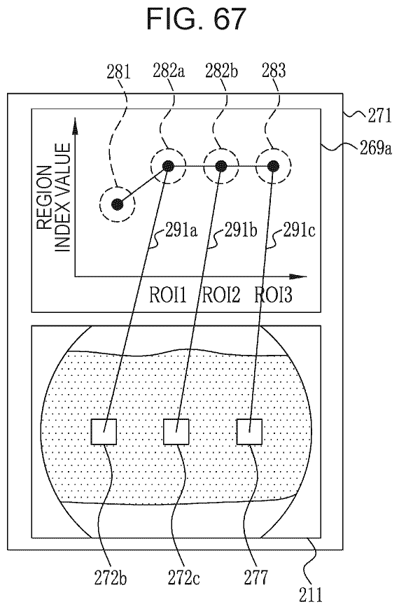

A specific example of the change of the display size of the index value display table such that adjacent region index values are displayed with the reduced distance therebetween will be described with reference to FIG. 67. FIG. 67 illustrates an extension index value display table 269a obtained by reducing the size of the extension index value display table 269 displayed in the display image 271 illustrated in FIG. 66 so as to reduce a distance between adjacent region index values. As described above, the change of the display size of the index value display table can improve the visibility of the index value display table. In addition, the display range of the index value display table can be reduced, for example, when the inch size of the display is small or when other information is to be displayed on the display image. In addition, the change of the display size of the index value display table is suitable for a case where the index value display table is to be displayed in a large size on the display. The control for changing the display size of the index value display table in the display image may be performed through an operation input by the user or may be performed automatically.

In FIG. 66, an example in which an index value link line is displayed in a display image that displays an out-of-field-of-view lock-on area index value and an additional region of interest is illustrated. The index value link line may be displayed in a display image that does not display an out-of-field-of-view lock-on area index value and an additional region of interest. A specific example will be described hereinafter.

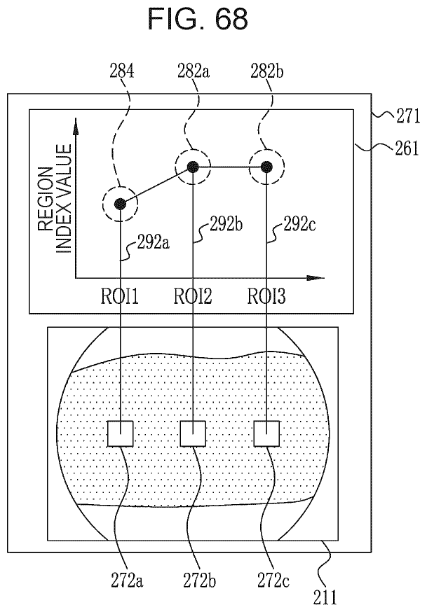

For example, to display an index value link line in the display image 271 illustrated in the example in FIG. 49, a display image 271 as illustrated in FIG. 68 is displayed on the display. In the example of the display image illustrated in FIG. 68, index value link lines 292a, 292b, and 292c are displayed in the display image 271 illustrated in the example in FIG. 49.

The index value link line 292a is an index value link line connecting a region index value 284 and the region position information 272a. The index value link line 292b is an index value link line connecting the region index value 282a and the region position information 272b. The index value link line 292c is an index value link line connecting the region index value 282b and the region position information 272c.

The index value link line 292a, the index value link line 292b, and the index value link line 292c are generated by the index value link line generation unit 290, based on the association stored in the region index value storage unit 280. In the example of the display image illustrated in FIG. 68, the region index value 284 and the region position information 272a are stored in the region index value storage unit 280 in association with each other, the region index value 282a and the region position information 272b are stored in the region index value storage unit 280 in association with each other, and the region index value 282b and the region position information 272c are stored in the region index value storage unit 280 in association with each other. The region position information 272a, the region position information 272b, and the region position information 272c are lock-on area position information, and the region index value 284, the region index value 282a, and the region index value 282b are lock-on area index values.

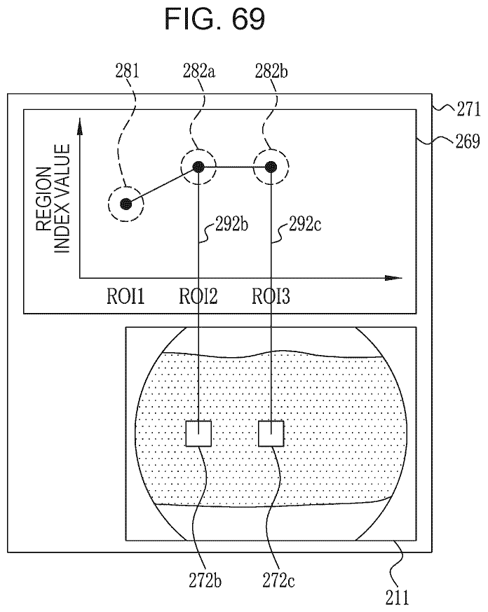

When, in the display image 271 as illustrated in FIG. 68, the region position information 272a (lock-on area position information) becomes at a position out of the field of view in response to the movement of the endoscope 12, a display image 271 as illustrated in FIG. 69 for displaying the extension index value display table 269 that displays the out-of-field-of-view lock-on area index value 281 is displayed. In this case, since the region position information 272a is at the position out of the field of view, the index value link line 292a displayed in FIG. 68 is not displayed in the display image 271 illustrated in FIG. 69.

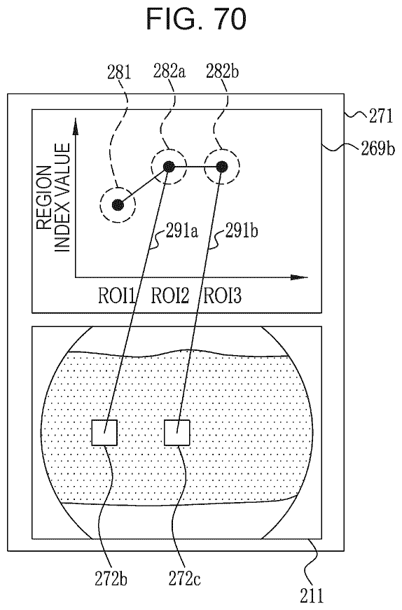

When, in the display image 271 as illustrated in FIG. 69, the display size of the index value display table is changed to reduce the size of the extension index value display table 269a by reducing a distance between adjacent region index values, an extension index value display table 269b as illustrated in FIG. 70, which is obtained by reducing the size of the extension index value display table 269a, is displayed in the display image 271.

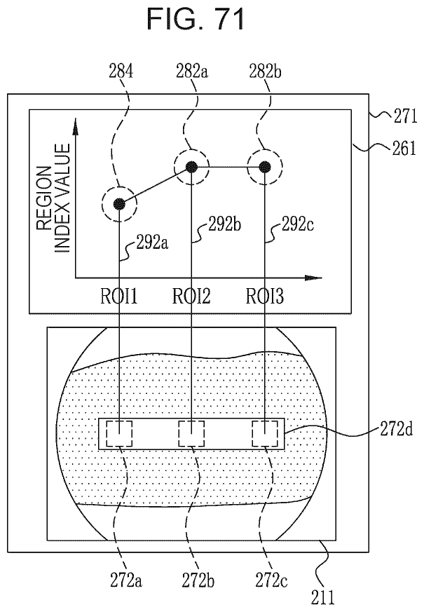

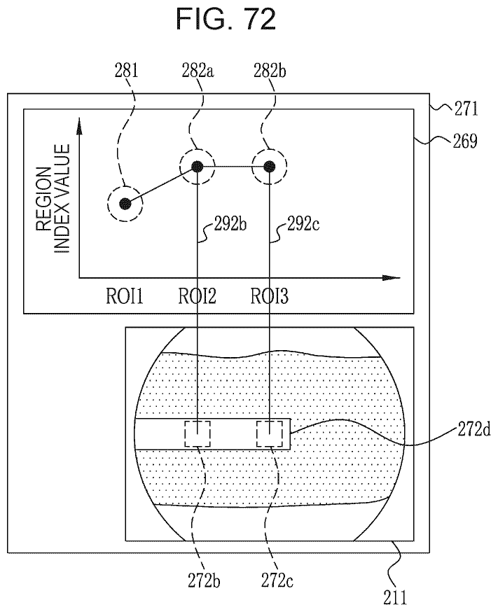

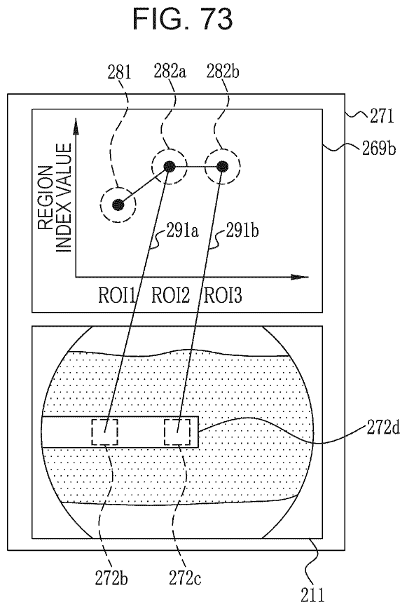

In the display image 271 illustrated in the examples in FIGS. 68, 69, and 70, display-region position information 272d indicated by a solid line as illustrated in FIGS. 71, 72, and 73 and including one or more of the pieces of region position information 272a, 272b, and 272c indicated by broken lines may be displayed. As illustrated in FIGS. 72 and 73, it is preferable to change the display mode of the display-region position information 272d when the lock-on area position information 272a follows the movement of the endoscope 12 and becomes at a position out of the field of view.

Further, an additional region of interest may also be additionally set in a display image that does not display an out-of-field-of-view lock-on area index value. Alternatively, the index value display table on the display image does not display an out-of-field-of-view lock-on area index value, but may display only a region index value stored in association with region position information displayed in the display image. A specific example will be described hereinafter.

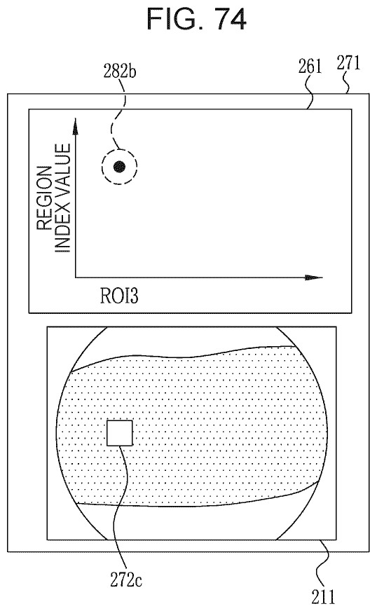

For example, when, in the display image 271 illustrated in the example in FIG. 49, the pieces of region position information 272a and 272b (lock-on area position information) become at a position out of the field of view in response to the movement of the endoscope 12 and, as illustrated in FIG. 74, only the region position information 272c (lock-on area position information) is displayed in the display image, only the region index value 282b associated with the region position information 272c may be displayed in the index value display table 261 on the display image 271.

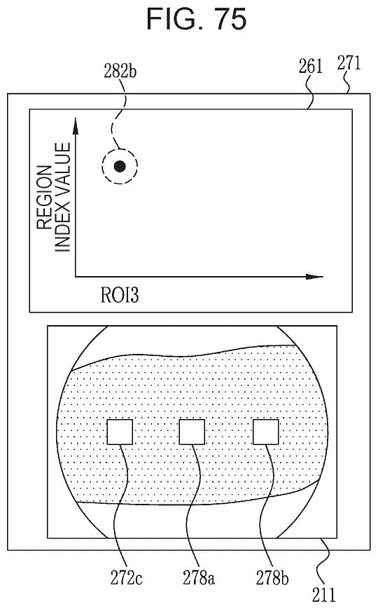

When the display image 271 as illustrated in FIG. 74 is displayed, in response to a region-of-interest setting instruction being input by a user operation, the region-of-interest setting unit 210 sets additional regions of interest 278a and 278b as illustrated in FIG. 75. In the example illustrated in FIG. 75, it is set in advance to set two additional regions of interest. The number of additional regions of interest to be set may be set as desired, and may be one or three or more. The additional regions of interest 278a and 278b in the example of the display image illustrated in FIG. 75 are, to be precise, pieces of region position information of the additional regions of interest 278a and 278b to be displayed in a superimposed manner on the display image.

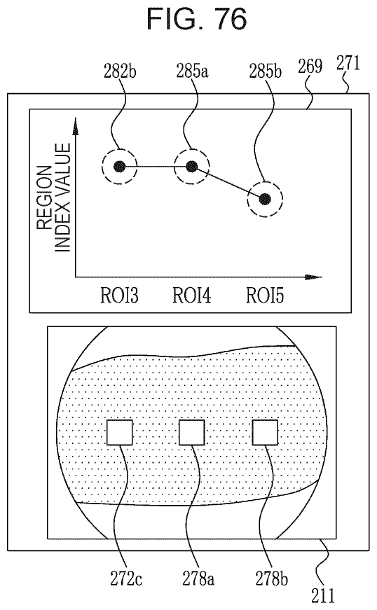

When the additional regions of interest 278a and 278b are set, the pieces of region position information of the additional regions of interest 278a and 278b are stored in the region position information storage unit 240. The region index value calculation unit 250 calculates additional region index values 285a and 285b (see FIG. 76) as statistical values of biological index values calculated based on image signals in the additional regions of interest 278a and 278b. The additional region index value 285a is a region index value calculated based on image signals in the additional region of interest 278a. The additional region index value 285b is a region index value calculated based on image signals in the additional region of interest 278b.

The index value display table generation unit 260 generates the extension index value display table 269, which is an index value display table that collectively displays the region index value 282b of the region position information 272c and the additional region index values 285a and 285b of the two lock-on areas displayed as the additional region of interest 278a and the additional region of interest 278b. The extension display control unit 200 performs control to display the display image generated by the display image generation unit 270 for displaying the extension index value display table 269, thereby displaying a display image 271 as illustrated in FIG. 76. The display image 271 as illustrated in FIG. 76 may also display an index value link line.

In FIG. 76, an example is illustrated in which the extension index value display table 269 that collectively displays the region index value 282b and the additional region index values 285a and 285b is displayed. Alternatively, an index value display table that displays the region index value 282b and an index value display table that displays the additional region index values 285a and 285b may be displayed separately as a plurality of different index value display tables.

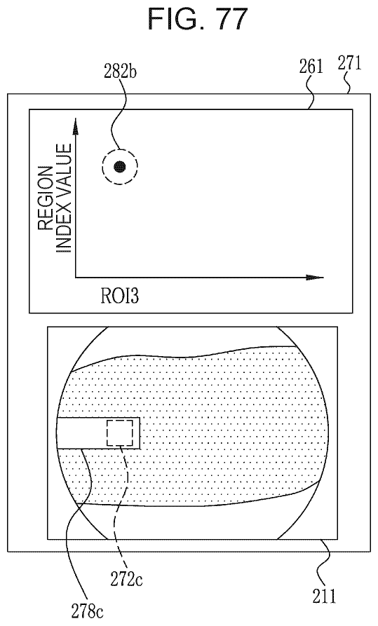

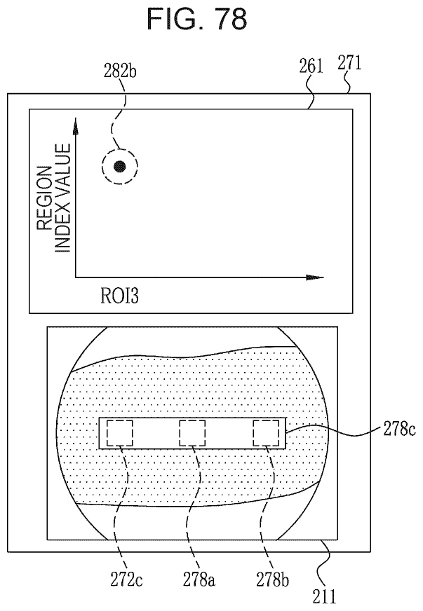

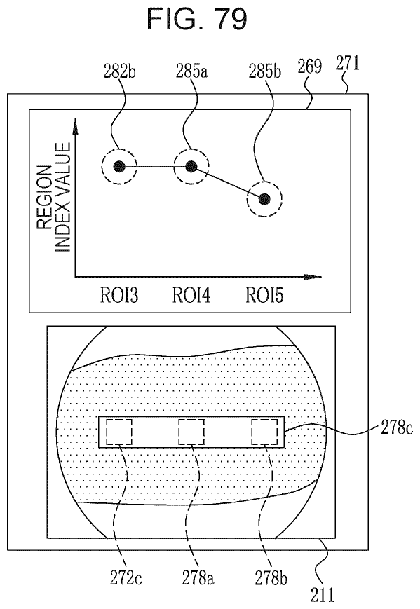

In the display image 271 illustrated in the examples in FIGS. 74, 75, and 76, display-region position information 278c indicated by a solid line as illustrated in FIGS. 77, 78, and 79 and including one or more of the pieces of region position information 272c, 278a, and 278b indicated by broken lines may be displayed. As illustrated in FIGS. 78 and 79, it is preferable to change the display mode of the display-region position information 278c when an additional region of interest is set.

As described above, the display of an additional region index value allows information in a wide range of the photographic subject to be presented to the user. In this way, region index values for a plurality of regions of interest are displayed, and a region of interest can be added. As a result, the display of spatial information of biological index values in a biological tissue can support the user in identifying a region suitable for incision.

### Second Embodiment

In a second embodiment, a broadband light source 400 that emits broadband light, such as a white LED, a xenon lamp, or a halogen light source, instead of the LEDs 20a to 20e of the respective colors presented in the first embodiment, is used in place of the light source unit 20, and the broadband light source 400 and a rotary filter 410 are combined to use the light emitted from the light source device 13 as illumination light for illuminating the photographic subject. Hereinafter, portions of the endoscope system 10 different from those according to the first embodiment will be described, and a description of common portions will be omitted.

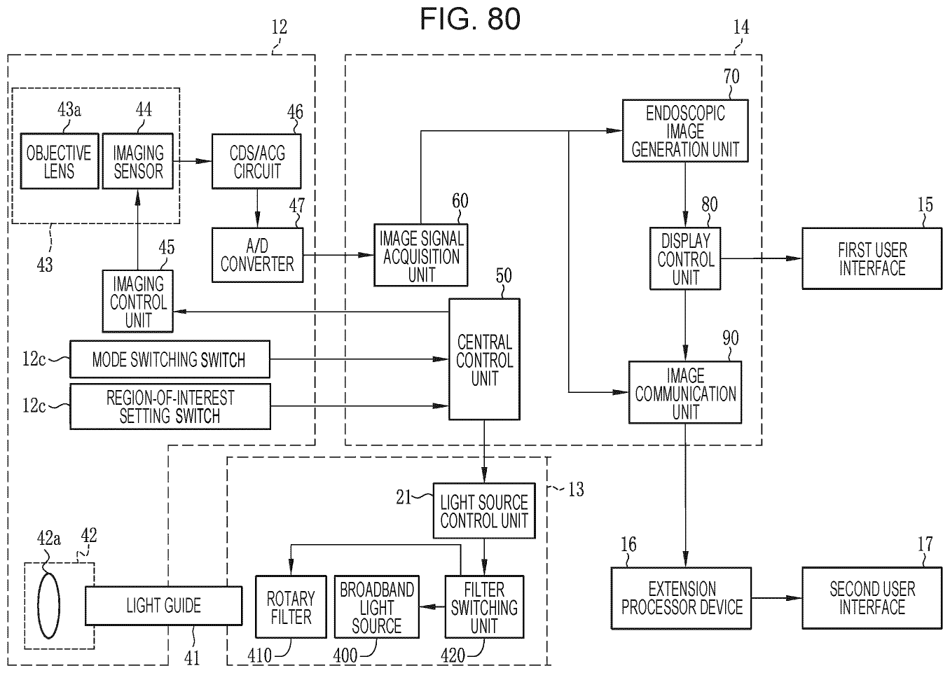

In the second embodiment, as illustrated in FIG. 80, the light source device 13 of the endoscope system 10 is provided with the broadband light source 400, the rotary filter 410, and a filter switching unit 420. The filter switching unit 420 is controlled by the light source control unit 21. The other components are similar to those of the endoscope system 10 according to the first embodiment. In the second embodiment, the imaging sensor 44 may be a monochrome imaging sensor.

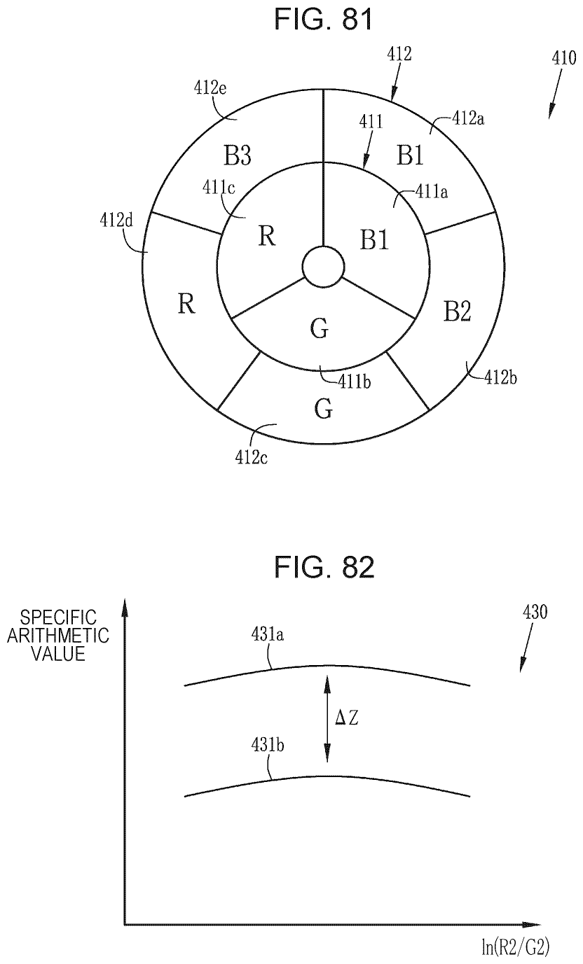

The broadband light source 400 emits broadband light having a wavelength range ranging from blue to red. The broadband light is, for example, white light. As illustrated in FIG. 81, the rotary filter 410 includes an inner filter 411 provided on the inner side and an outer filter 412 provided on the outer side. The filter switching unit 420 moves the rotary filter 410 in the radial direction. In the normal mode, the filter switching unit 420 inserts the inner filter 411 of the rotary filter 410 into an optical path of white light. In the oxygen saturation mode or the correction mode, the filter switching unit 420 inserts the outer filter 412 of the rotary filter 410 into an optical path of white light.

As illustrated in FIG. 81, the inner filter 411 is provided with, in the circumferential direction thereof, a B1 filter 411a that transmits the wavelength ranges of the violet light V and the second blue light BS of the white light, a G filter 411b that transmits the wavelength range of the green light G of the white light, and an R filter 411c that transmits the wavelength range of the red light R of the white light. In the normal mode, accordingly, illumination light having the wavelength ranges of the violet light V and the second blue light BS, illumination light having the wavelength range of the green light G, and illumination light having the wavelength range of the red light R are emitted from the light source device 13 in accordance with the rotation of the rotary filter 410.

As illustrated in FIG. 81, the outer filter 412 is provided with, in the circumferential direction thereof, a B1 filter 412a that transmits the first blue light BL of the white light having the wavelength range B1, a B2 filter 412b that transmits light of the wavelength range of the second blue light BS of the white light, a G filter 412c that transmits the green light G of the white light having the wavelength range G2, an R filter 412d that transmits the red light R of the white light having the wavelength range R2, and a B3 filter 412e that transmits blue-green light BG of the white light as light of the wavelength range B3 (see FIG. 21 and FIGS. 22A and 22B). In the oxygen saturation mode or the correction mode, accordingly, illumination light having the wavelength ranges of the first blue light BL, the second blue light BS, the green light G, the red light R, and the blue-green light BG is emitted from the light source device 13 in accordance with the rotation of the rotary filter 410.

In the endoscope system 10, in the normal mode, reflected light obtained by illuminating the photographic subject with the illumination light having the wavelength ranges of the violet light V and the second blue light BS is captured using the monochrome imaging sensor to output the Bc image signal. Further, reflected light obtained by illuminating the photographic subject with the illumination light having the wavelength range of the green light G is captured using the monochrome imaging sensor to output the Gc image signal. Further, reflected light obtained by illuminating the photographic subject with the illumination light having the wavelength range of the red light R is captured using the monochrome imaging sensor to output the Rc image signal. Next, a white-light image is generated by a method similar to that in the first embodiment, based on the Bc image signal, the Gc image signal, and the Rc image signal.

In the oxygen saturation mode or the correction mode, in contrast, reflected light obtained by illuminating the photographic subject with illumination light having the wavelength range of the first blue light BL is captured using the monochrome imaging sensor to output the B1 image signal. Further, reflected light obtained by illuminating the photographic subject with illumination light having the wavelength range of the second blue light BS is captured using the monochrome imaging sensor to output the B2 image signal. Further, reflected light obtained by illuminating the photographic subject with the illumination light having the wavelength range of the green light G is captured using the monochrome imaging sensor to output the G2 image signal. Further, reflected light obtained by illuminating the photographic subject with the illumination light having the wavelength range of the red light R is captured using the monochrome imaging sensor to output the R2 image signal. Further, reflected light obtained by illuminating the photographic subject with illumination light having the wavelength range of the blue-green light BG is captured using the monochrome imaging sensor to output the B3 image signal. Next, the extension processor device 16 generates an oxygen saturation image by a method similar to that in the first embodiment, based on the B1 image signal, the B2 image signal, the G2 image signal, the R2 image signal, and the B3 image signal transmitted from the processor device 14. In addition, correction processing is performed. However, in the second embodiment, a signal ratio ln(B3/G2) obtained by normalizing the B3 image signal by the G2 image signal is used instead of the signal ratio ln(B3/G3).

As the correction processing related to the calculation of the oxygen saturation in the correction mode, table correction processing for referring to the corrected oxygen saturation calculation table 120 to select an oxygen saturation calculation table corresponding to a specific pigment concentration and setting the oxygen saturation calculation table as the selected oxygen saturation calculation table may be performed. Alternatively, calculation value correction processing for adding or subtracting a correction value obtained from a specific arithmetic value to or from the oxygen saturation calculated by referring to the oxygen saturation calculation table 110 as illustrated in FIG. 24 may be performed.

In the calculation value correction processing, a correction value used for the correction of the oxygen saturation is calculated by referring to a two-dimensional coordinate system 430 illustrated in FIG. 82. The vertical axis of the two-dimensional coordinate system 430 represents a specific arithmetic value obtained on the basis of the B1 image signal, the G2 image signal, the R2 image signal, and the B3 image signal, and the horizontal axis thereof represents ln(R2/G2). The specific arithmetic value is determined by Expression (A) below.

B1/G2×cos φ−B3/G2×sin φ Expression (A)

The two-dimensional coordinate system 430 presents a reference line 431a indicating the distribution of predetermined reference baseline information and an actual measurement line 431b indicating the distribution of actual measurement baseline information obtained by actual imaging of the observation target. A difference value AZ between the reference line 431a and the actual measurement line 431b is calculated as a correction value. The reference baseline information is obtained in the absence of the specific pigment and is determined as information independent of the oxygen saturation. Specifically, a value obtained by adjusting q so that Expression (A) described above is kept constant even when the oxygen saturation changes is set as the reference baseline information.

The oxygen saturation may be calculated by referring to the three-dimensional coordinate system 121 illustrated in FIG. 34. In the second embodiment, the Z-axis of the three-dimensional coordinate system 121 illustrated in FIG. 34 represents the signal ratio ln(B3/G2), and the signal ratio ln(B3/G2) is used as the Z-component value.

### Third Embodiment

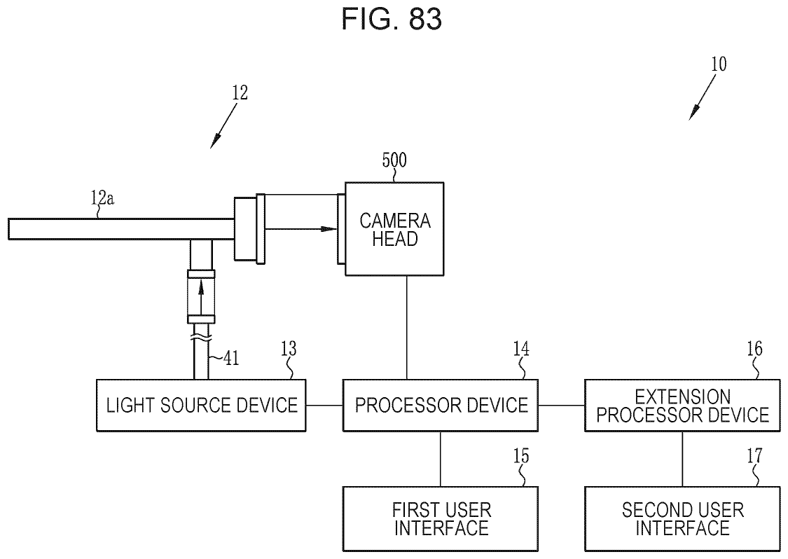

In a third embodiment, the endoscope 12 is a rigid endoscope as illustrated in FIG. 83 in which the proximal end portion of the insertion section 12a includes a camera head 500. The camera head 500 includes the imaging optical system 43. In the first embodiment and the second embodiment, the imaging optical system 43 having the objective lens 43a and the imaging sensor 44 is provided in the tip portion of the endoscope 12. In the third embodiment, however, the imaging sensor of the imaging optical system 43 is included in the camera head 500 instead of the tip portion of the endoscope 12. The camera head 500 captures reflected light guided from the tip portion of the endoscope 12. An image signal captured by the camera head 500 is transmitted to the processor device 14. In FIG. 83, the mode switching switch 12c and the region-of-interest setting switch 12d are omitted. Hereinafter, portions of the endoscope system 10 different from those according to the first embodiment and the second embodiment will be described, and a description of common portions will be omitted.

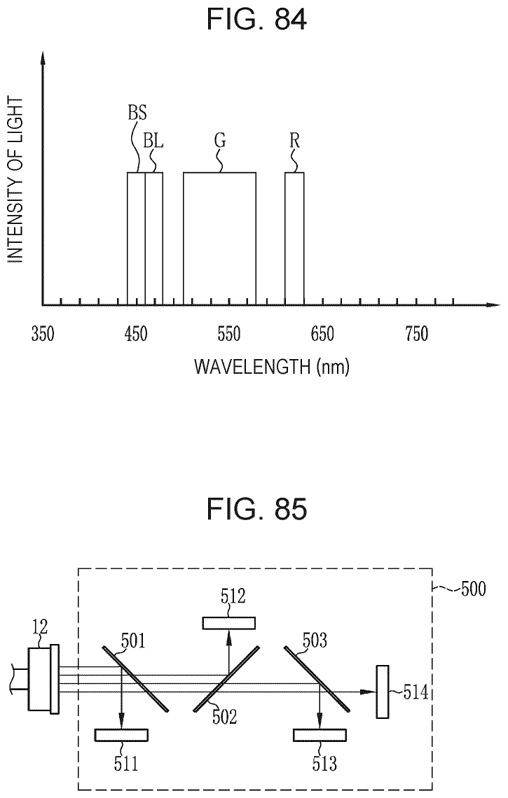

In the normal mode, the light source device 13 emits white light including the violet light V, the second blue light BS, the green light G, and the red light R. In the oxygen saturation mode and the correction mode, the light source device 13 emits illumination light as illustrated in FIG. 84, which is mixed light including the first blue light BL, the second blue light BS, the green light G, and the red light R.

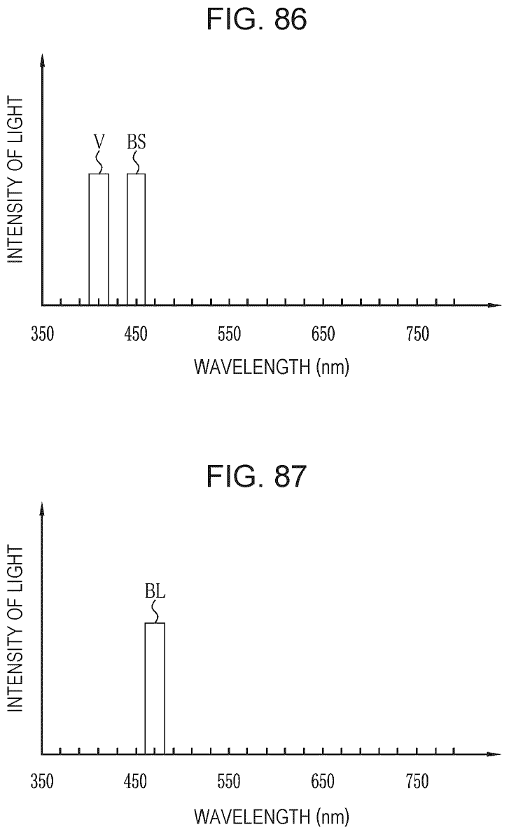

As illustrated in FIG. 85, the camera head 500 includes dichroic mirrors 501, 502, and 503, and imaging sensors 511, 512, 513, and 514, which are monochrome imaging sensors. The dichroic mirror 501 reflects light in the wavelength ranges of the violet light V and the second blue light BS from the reflected light from the photographic subject, and transmits light in the wavelength ranges of the first blue light BL, the green light G, and the red light R from the reflected light from the photographic subject. The light reflected by the dichroic mirror 501 and incident on the imaging sensor 511 has the wavelength range of the violet light V or the second blue light BS, as illustrated in FIG. 86. The imaging sensor 511 outputs the Bc image signal in the normal mode, and outputs the B2 image signal in the oxygen saturation mode or the correction mode.

The dichroic mirror 502 reflects light in the wavelength range of the first blue light BL from the light transmitted through the dichroic mirror 501, and transmits light in the wavelength ranges of the green light G and the red light R from the light transmitted through the dichroic mirror 501. The light reflected by the dichroic mirror 502 and incident on the imaging sensor 512 has the wavelength range of the first blue light BL, as illustrated in FIG. 87. The imaging sensor 512 stops outputting an image signal in the normal mode, and outputs the B1 image signal in the oxygen saturation mode or the correction mode.

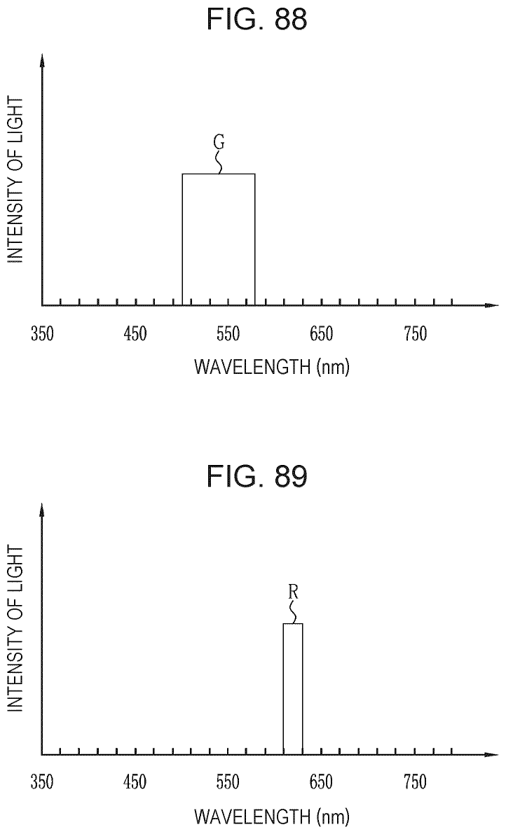

The dichroic mirror 503 reflects light in the wavelength range of the green light G from the light transmitted through the dichroic mirror 502, and transmits light in the wavelength range of the red light R from the light transmitted through the dichroic mirror 502. The light reflected by the dichroic mirror 503 and incident on the imaging sensor 513 has the wavelength range of the green light G, as illustrated in FIG. 88. The imaging sensor 513 outputs the Gc image signal in the normal mode, and outputs the G2 image signal in the oxygen saturation mode or the correction mode.

The light transmitted through the dichroic mirror 503 and incident on the imaging sensor 514 has the wavelength range of the red light R, as illustrated in FIG. 89. The imaging sensor 514 outputs the Rc image signal in the normal mode, and outputs the R2 image signal in the oxygen saturation mode or the correction mode.

That is, in the third embodiment, the Bc image signal, the Gc image signal, and the Rc image signal are output from the camera head in the normal mode, and the B1 image signal, the B2 image signal, the G2 image signal, and the R2 image signal are output from the camera head in the oxygen saturation mode or the correction mode. The Bc image signal, the Gc image signal, and the Rc image signal output from the camera head are acquired by the image signal acquisition unit 60 of the processor device 14, and are transmitted to the endoscopic image generation unit 70 to generate a white-light image. The B1 image signal, the B2 image signal, the G2 image signal, and the R2 image signal output from the camera head are acquired by the image signal acquisition unit 60 of the processor device 14. The B2 image signal, the G2 image signal, and the R2 image signal are transmitted to the endoscopic image generation unit 70 to generate a white-light-equivalent image. The B1 image signal, the B2 image signal, the G2 image signal, and the R2 image signal are transmitted to the extension processor device 16 via the image communication unit 90 to generate an oxygen saturation image.

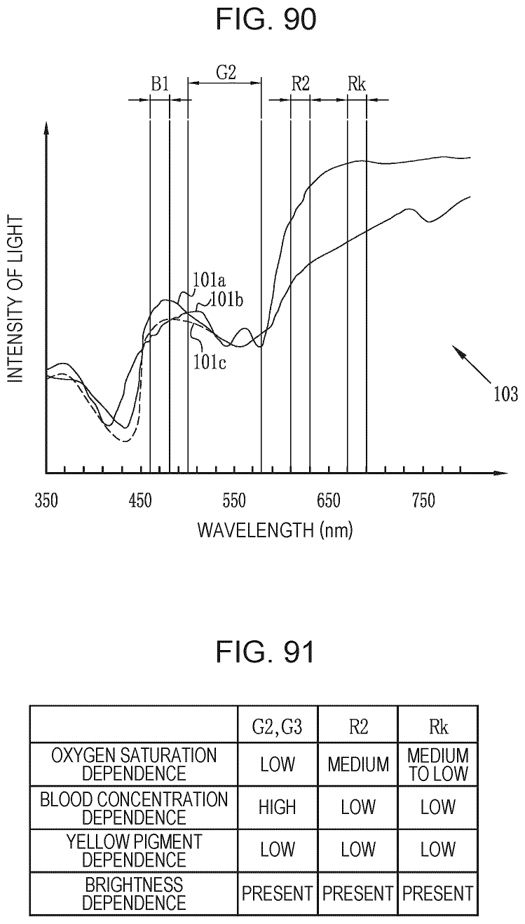

In the first and second embodiments, the B1 image signal including the information on the wavelength range B1 is used to calculate the oxygen saturation. However, another image signal may be used instead of the B1 image signal. For example, as illustrated in FIG. 90 (see FIG. 22A), an Rk image signal including information on a wavelength range Rk in which the difference in reflection spectrum between deoxyhemoglobin (Hb) and oxyhemoglobin (HbO₂) is large may be used. As illustrated in FIG. 90, the wavelength range Rk is a wavelength range in the range of 680 nm+10 nm. As illustrated in FIG. 91, for the Rk image signal (indicated by “Rk” in FIG. 91), the oxygen saturation dependence is “medium to low”, the blood concentration dependence is “low”, the yellow pigment dependence is “low”, and the brightness dependence is “present”. To output the Rk image signal, the camera head 500 includes an imaging sensor capable of detecting the wavelength range Rk and a dichroic mirror that reflects light in the wavelength range of the red light R transmitted through the dichroic mirror 503 and transmits light having the wavelength range Rk.

### Fourth Embodiment

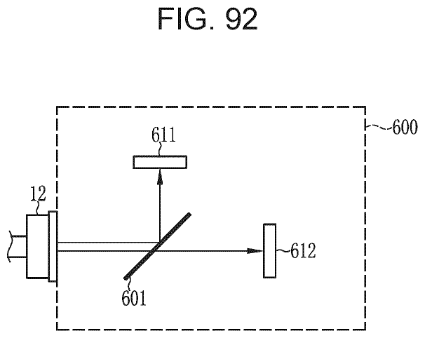

In a fourth embodiment, as in the third embodiment, the endoscope 12 is a rigid endoscope in which the proximal end portion of the insertion section 12a includes a camera head. Hereinafter, portions different from the first embodiment, the second embodiment, and the third embodiment will be described, and a description of common portions will be omitted. In the fourth embodiment, a camera head 600 as illustrated in FIG. 92 is included instead of the camera head 500 according to the third embodiment.

As illustrated in FIG. 92, the camera head 600 includes a dichroic mirror 601 and imaging sensors 611 and 612. The dichroic mirror 601 reflects light in the wavelength ranges of the violet light V, the second blue light BS, the green light G, and the red light R from the reflected light from the photographic subject, and transmits light in the wavelength range of the first blue light BL from the reflected light from the photographic subject.

The imaging sensor 611, which receives the light reflected by the dichroic mirror 601, is a color imaging sensor in which a B color filter BF is provided in a B pixel, a G color filter GF is provided in a G pixel, and an R color filter RF is provided in an R pixel. The imaging sensor 612, which receives the light transmitted through the dichroic mirror 601, is a monochrome imaging sensor.

In the normal mode, white light is emitted from the light source device 13 (see FIG. 10), and the imaging sensor 611 as a color imaging sensor receives reflected light from the photographic subject, which is reflected by the dichroic mirror 601. As a result, the Bc image signal, the Gc image signal, and the Rc image signal are output from the imaging sensor 611. In the normal mode, the imaging sensor 612 as a monochrome imaging sensor stops outputting an image signal.

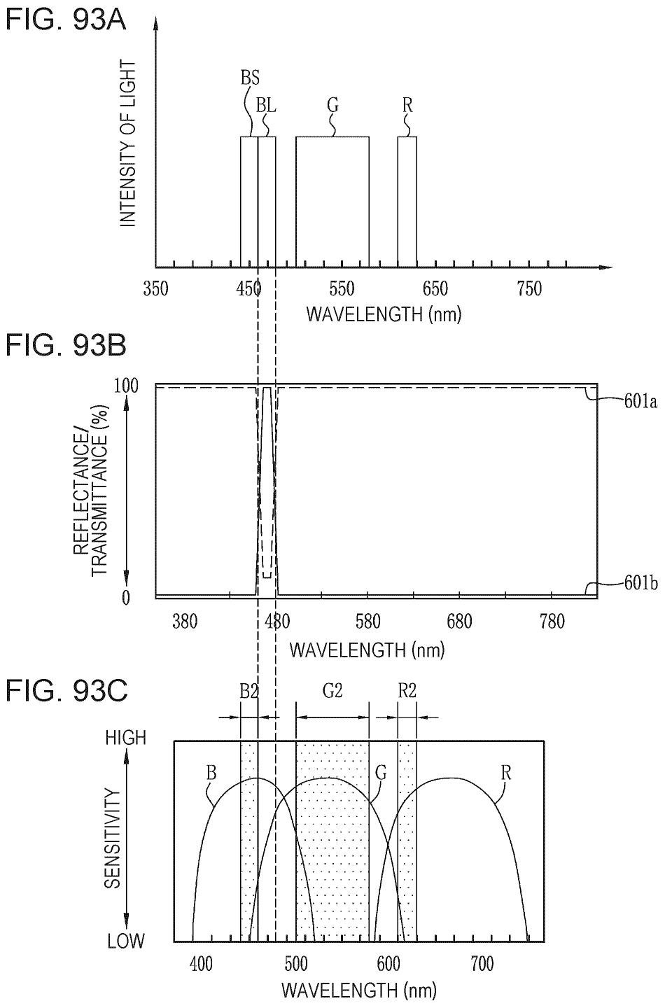

In the oxygen saturation mode, observation illumination light (hereinafter referred to as fourth illumination light) as illustrated in FIG. 93A including the second blue light BS, the first blue light BL, the green light G, and the red light R is emitted from the light source device 13. The dichroic mirror 601 reflects and transmits the reflected light from the photographic subject illuminated with the fourth illumination light to spectrally separate the reflected light. FIG. 93B illustrates the relationship between the reflectance (broken line 601a) and transmittance (solid line 601b) of light incident on the dichroic mirror 601 and the wavelengths of the light.

Of the reflected light from the photographic subject illuminated with the fourth illumination light, the light reflected by the dichroic mirror 601 is received by the imaging sensor 611 as a color imaging sensor. The sensitivities of a B pixel B, a G pixel G, and an R pixel R of the imaging sensor 611 and the wavelengths of light have a relationship as illustrated in FIG. 93C. Accordingly, the B pixel B of the imaging sensor 611 senses light in the wavelength range B2 of the second blue light BS to output the B2 image signal. The G pixel G of the imaging sensor 611 senses light in the wavelength range G2 of the green light G to output the G2 image signal. The R pixel R of the imaging sensor 611 senses light in the wavelength range R2 of the red light R to output the R2 image signal.

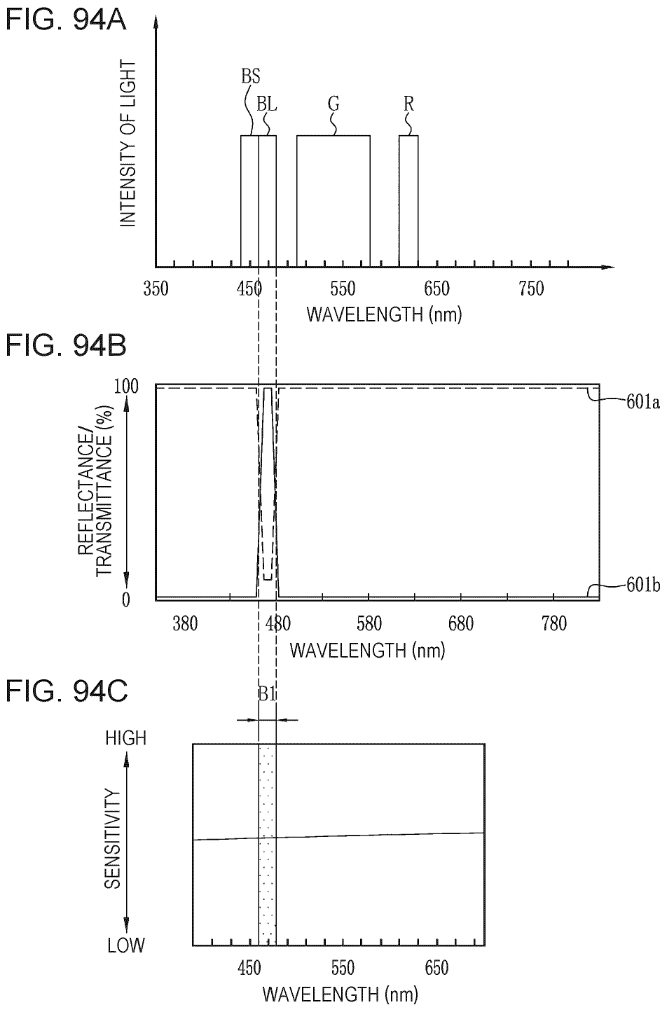

In contrast, of the reflected light from the photographic subject illuminated with the fourth illumination light, the light transmitted through the dichroic mirror 601 is received by the imaging sensor 612 as a monochrome imaging sensor. The sensitivity of the imaging sensor 612 and the wavelengths of light have a relationship illustrated in FIG. 94C. Accordingly, the imaging sensor 612 senses the light in the wavelength range BL of the first blue light BL transmitted through the dichroic mirror 601 to output the B1 image signal. Like FIG. 93A, FIG. 94A illustrates the wavelength ranges of light included in the fourth illumination light. Like FIG. 93B, FIG. 94B illustrates the relationship between the reflectance (broken line 601a) and transmittance (solid line 601b) of light incident on the dichroic mirror 601 and the wavelengths of the light.

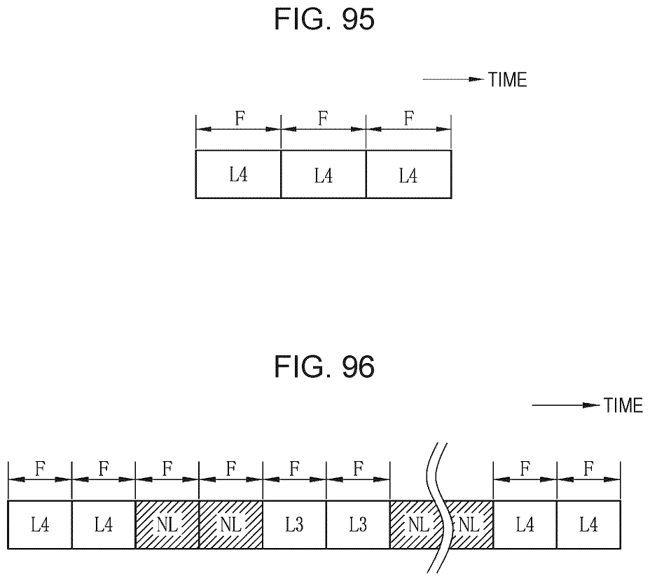

In the oxygen saturation mode according to the fourth embodiment, as illustrated in FIG. 95, a light emission pattern in which fourth illumination light L4 is emitted once for each frame F is repeated. In the oxygen saturation mode according to the fourth embodiment, accordingly, for each frame, the B2 image signal, the G2 image signal, and the R2 image signal are output from the imaging sensor 611 as a color imaging sensor, and the B1 image signal is output from the imaging sensor 612 as a monochrome imaging sensor. In the oxygen saturation mode, the B1 image signal, the B2 image signal, the G2 image signal, and the R2 image signal output from the imaging sensor 611 or the imaging sensor 612 are transmitted to the processor device 14. The method for calculating the biological index value including the oxygen saturation in the oxygen saturation mode is similar to that in the first embodiment.

In the correction mode according to the fourth embodiment, as illustrated in FIG. 96, when a light emission switching instruction is input by a user operation during the emission of the fourth illumination light L4, a light emission pattern is taken in which the third illumination light L3 is emitted for two frames F after a non-light emission state NL for two frames F and the fourth illumination light L4 is emitted for two frames F after the non-light emission state NL for a plurality of frames. The frames corresponding to the non-light emission state NL are a period of time for switching between the fourth illumination light L4 and the third illumination light L3, and no illumination light is emitted in this period of time. The light emission switching instruction may be input by an operation of an illumination light switching switch (not illustrated) provided in the endoscope 12 or the user interface, or may be input by a toggle operation of the mode switching switch 12c.

In the correction mode, in the frame in which the fourth illumination light L4 is emitted, as in the oxygen saturation mode, the B2 image signal, G2 image signal, and the R2 image signal are output from the imaging sensor 611 and the B1 image signal is output from the imaging sensor 612.

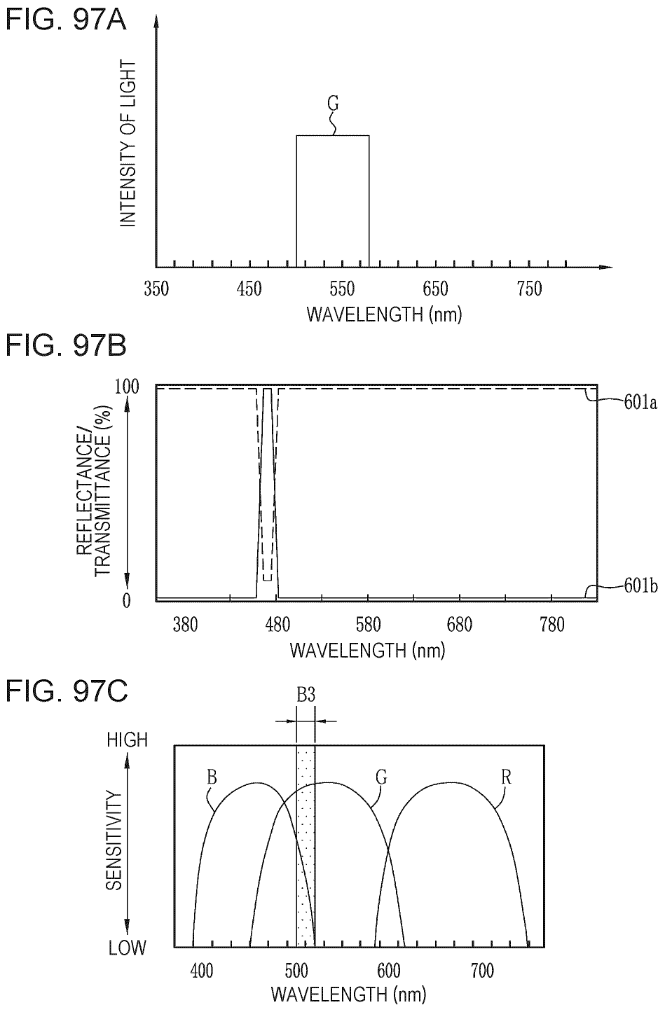

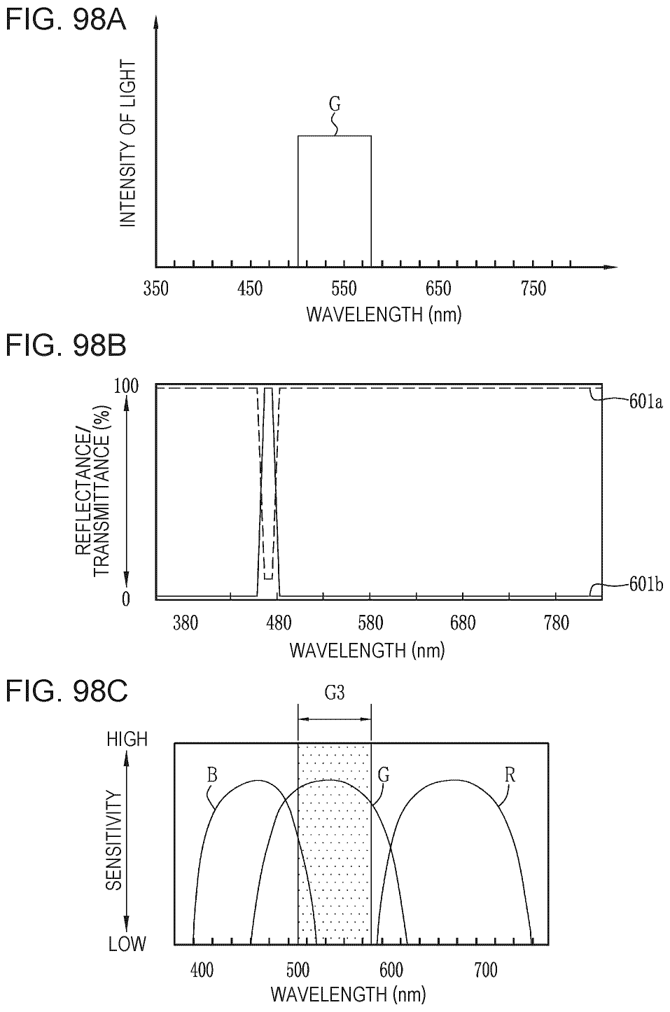

In the correction mode, in the frame in which the third illumination light L3 is emitted, the third illumination light (correction illumination light) as illustrated in FIG. 97A including the green light G is emitted from the light source device 13. Further, the reflected light from the photographic subject reflected by the dichroic mirror 601 (see FIG. 97B) is received by the imaging sensor 611 as a color imaging sensor. As a result, as illustrated in FIG. 97C, the B pixel B of the imaging sensor 611 senses light in the wavelength range B3 of the green light G to output the B3 image signal. In addition, as illustrated in FIG. 98C, the G pixel G of the imaging sensor 611 senses light in the wavelength range G3 of the green light G to output the G3 image signal. Further, the R pixel R of the imaging sensor 611 senses light in a wavelength range of the green light G to output the R3 image signal (not illustrated).

Like FIG. 97A, FIG. 98A illustrates a wavelength range of light included in the third illumination light. Like FIG. 93B, FIGS. 97B and 98B illustrate the relationship between the reflectance (broken line 601a) and transmittance (solid line 601b) of light incident on the dichroic mirror 601 and the wavelengths of the light. Like FIG. 93C, FIGS. 97C and 98C illustrate the relationship between the sensitivities of the B pixel B, the G pixel G, and the R pixel R of the imaging sensor 611 and the wavelengths of light.

In the oxygen saturation mode, the B1 image signal, the B2 image signal, the G2 image signal, and the R2 image signal output from the imaging sensor 611 or the imaging sensor 612 are transmitted to the processor device 14 and acquired by the image signal acquisition unit 60. In the fourth embodiment, unlike the first and second embodiments, the image signal acquisition unit 60 performs demosaicing on the Bc image signal, the Gc image signal, and the Rc image signal acquired in the normal mode, the B2 image signal, the G2 image signal, and the R2 image signal acquired in the oxygen saturation mode and the correction mode, and the B3 image signal, the G3 image signal, and the R3 image signal acquired in the correction mode. The calculation of the reliability according to the fourth embodiment will be described hereinafter.

In the first embodiment, the correction image 161 is displayed, and the reliability of the specific region 162 included in the correction image 161 is calculated for each pixel included in the specific region 162. In the fourth embodiment, unlike the first embodiment, the reliability is calculated for each correction region by using an image (white-light-equivalent image and first blue light image) obtained in a frame in which the fourth illumination light L4 is emitted and an image (third illumination light image) obtained in a frame in which the third illumination light L3 is emitted. The correction region corresponds to the specific region in the first embodiment. The term “correction region” is used as a term indicating “a set of a plurality of divided sub-regions” or “a sub-region itself (N-th correction region, where N is a natural number of 1 or more)”, which will be described below.

The white-light-equivalent image is an endoscopic image generated using the B2 image signal, the G2 image signal, and the R2 image signal output in a frame in which the fourth illumination light L4 is emitted. The first blue light image is an endoscopic image generated using the B1 image signal output in a frame in which the fourth illumination light L4 is emitted. The third illumination light image is an endoscopic image generated using the B3 image signal, the G3 image signal, and the R3 image signal output in a frame in which the third illumination light L3 is emitted. In the fourth embodiment, the white-light-equivalent image and the third illumination light image are endoscopic images that are generated by the image signal acquisition unit 60 performing demosaicing and in which all the pixels have pixel values. Since the first blue light image is output from a monochrome image sensor, all the pixels have pixel values at the time point when the image signal acquisition unit 60 acquires the B1 image signal.

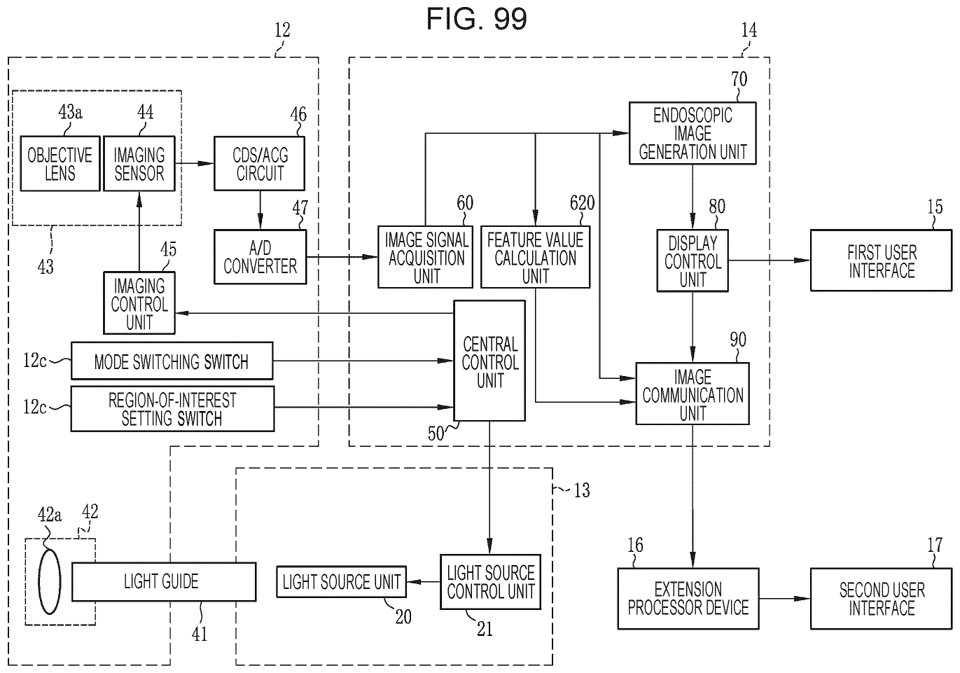

The white-light-equivalent image and the third illumination light image are transmitted to a feature value calculation unit 620 of the processor device 14 illustrated in FIG. 99. The feature value calculation unit 620 may be constituted by a processor different from that of the central control unit 50 in the processor device 14. For example, the feature value calculation unit 620 may be constituted by a field programmable gate array (FPGA).

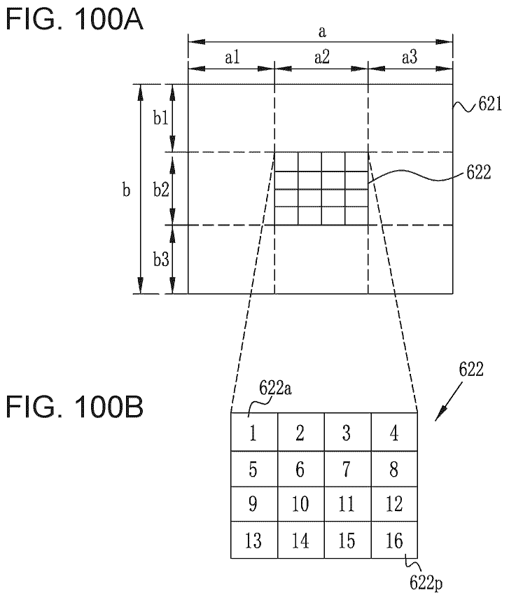

The feature value calculation unit 620 calculates a region feature value for each of a plurality of correction regions illustrated in FIGS. 100A and 100B in the white-light-equivalent image, the first blue light image, and the third illumination light image. The region feature value will be described below. As illustrated in FIGS. 100A and 100B, a correction region 622 is a sub-region obtained by dividing a white-light-equivalent image 621 into a plurality of regions. In the example illustrated in FIGS. 100A and 100B, a horizontal length a of the white-light-equivalent image 621 is equally divided into three sections a1, a2, and a3, a vertical length b of the white-light-equivalent image 621 is equally divided into three sections b1, b2, and b3, and a region where the column corresponding to the section a2 and the row corresponding to the section b2 overlap, which is the correction region 622, is further equally divided into 16 regions (see FIG. 100A). The positions of the regions of the correction region 622 and the number of regions divided from the correction region 622 are not limited to those described above. For example, the correction region 622 may be equally divided into 9 sections or equally divided into 25 sections.

FIG. 100B is an enlarged view of the correction region 622 illustrated in FIG. 100A. As illustrated in FIG. 100B, the correction region 622 is a region divided into 16 regions, namely, a first correction region 622a to a sixteenth correction region 622p. In FIG. 100B, the 16 regions, namely, the first correction region 622a to the sixteenth correction region 622p, divided from the correction region 622 are assigned numerals from 1 to 16.

The feature value calculation unit 620 determines whether the pixels of each correction region are effective pixels for each correction region of the N correction regions (in the example illustrated in FIGS. 100A and 100B, the first correction region to the sixteenth correction region). The determination of whether the pixels are effective pixels is performed by providing lower limit and upper limit channel threshold values for each channel (B channel, G channel, and R channel) of each pixel.

A B channel lower limit threshold value and a B channel upper limit threshold value are provided for the B channel. A G channel lower limit threshold value and a G channel upper limit threshold value are provided for the G channel. An R channel lower limit threshold value and an R channel upper limit threshold value are provided for the R channel.

When the pixel values for the channels of all the colors of each pixel in the correction regions of the white-light-equivalent image, the first blue light image, and the third illumination light image are each in a range greater than or equal to the channel lower limit threshold value and less than the channel upper limit threshold value for the corresponding color, the feature value calculation unit 620 determines that the pixel is an effective pixel.

Each pixel of the white-light-equivalent image and the third illumination light image is determined to be an effective pixel when the pixel has a B-channel pixel value in the range greater than or equal to the B-channel lower limit threshold value and less than the B-channel upper limit threshold value, a G-channel pixel value in the range greater than or equal to the G-channel lower limit threshold value and less than the G-channel upper limit threshold value, and an R-channel pixel value in the range greater than or equal to the R-channel lower limit threshold value and less than the R-channel upper limit threshold value.

Each pixel of the first blue light image is determined to be an effective pixel when the pixel has a pixel value in the range greater than or equal to the monochrome-image channel lower limit value and less than the monochrome-image channel upper limit value.

Next, the feature value calculation unit 620 calculates a region feature value for each correction region in the white-light-equivalent image, the third illumination light image, and the first blue light image. The region feature value is the number of effective pixels, the sum of the pixel values of the effective pixels, the sum of the squares of the pixel values of the effective pixels, the variance of the pixel values of the effective pixels, or the like.

That is, the feature value calculation unit 620 calculates the region feature value for each correction region of each channel of the white-light-equivalent image. The feature value calculation unit 620 further calculates the region feature value for each correction region of each channel of the third illumination light image. The feature value calculation unit 620 further calculates the region feature value for each correction region of the first blue light image. The region feature value of each correction region of each channel of the respective endoscopic images calculated by the feature value calculation unit 620 is transmitted to the reliability calculation unit 160 of the extension processor device 16.

In the fourth embodiment, the reliability calculation unit 160 calculates the reliability for determining the degree of influence of disturbances on the correction region. The reliability calculation unit 160 further calculates a second pigment value for determining the degree of movement of the endoscope 12. The degree of movement of the endoscope 12 is a degree for determining whether the endoscope 12 has been moved during switching of the illumination light (that is, in the non-light emission state NL) in the correction mode according to the fourth embodiment. When the endoscope 12 moves in the non-light emission state NL, the observation target appearing in the endoscopic image also moves, possibly resulting in inappropriate correction processing. Accordingly, as will be described below, the degree of movement of the endoscope 12 in the correction region is calculated to make a determination on the movement of the endoscope 12 according to the degree of movement of the endoscope 12. If the degree of movement of the endoscope 12 is large, the user can be notified not to move the endoscope 12. The calculation of the second pigment value will be described below. In the fourth embodiment, the correction determination unit 170 of the extension processor device 16 determines the degree of influence of disturbances using the reliability and/or determines the degree of movement of the endoscope 12 using the second pigment value.

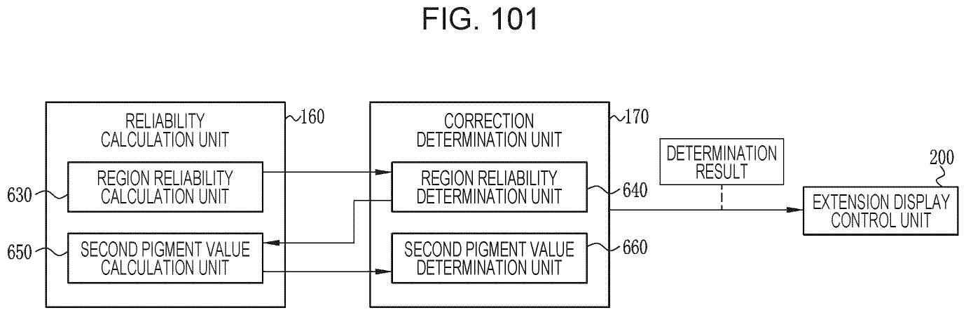

A method for determining the degree of influence of disturbances and a method for determining the degree of movement of the endoscope 12 according to the fourth embodiment will be described hereinafter. In the fourth embodiment, as illustrated in FIG. 101, the reliability calculation unit 160 has a region reliability calculation unit 630 and a second pigment value calculation unit 650. The correction determination unit 170 has a region reliability determination unit 640 and a second pigment value determination unit 660.

The region reliability calculation unit 630 of the reliability calculation unit 160 calculates the region reliability using the region feature value of each correction region of each channel of the white-light-equivalent image, the first blue light image, and the third illumination light image generated from the image signals output in each frame. The region reliability includes, for example, the mean value of the pixel values in the correction region, the standard deviation of the pixel values in the correction region, the effective pixel ratio in the correction region, the reliability regarding the brightness in the correction region, the reliability based on the degree of bleeding included in the correction region, and the reliability based on the degree of fat included in the correction region. The region reliability is a form of the “reliability” in the first embodiment.

The mean value of the pixel values in the correction region is calculated using the number of pixels in the correction region and the pixel values of the effective pixels in the correction region. The standard deviation of the pixel values in the correction region is calculated using the number of pixels in the correction region and the variance of the pixel values of the effective pixels. The effective pixel ratio in the correction region is calculated using the number of pixels in the correction region and the number of effective pixels. The reliability regarding the brightness in the correction region is calculated by applying the mean value of the G2 image signal in the correction region (i.e., a signal value obtained by converting the pixel values, in the correction region, of the G channel of the white-light-equivalent image) to a first reliability calculation table 763 as illustrated in FIG. 102 obtained by setting the signal value of the G2 image signal on the horizontal axis of the first reliability calculation table 163 (see FIG. 36). The signal value of the G2 image signal may be a mean value of brightness values obtained by performing a conversion process using the G2 image signal.

The reliability based on the degree of bleeding included in the correction region is calculated by calculating a region mean signal ratio ln(R2/G2) and a region mean signal ratio ln(B2/G2) using the mean value of the B2 image signal, the mean value of the G2 image signal, and the mean value of the R2 image signal in each correction region of the white-light-equivalent image (i.e., the means of the pixel values, in each correction region, of the respective color channels of the white-light-equivalent image) and applying these signal ratios to the second reliability calculation table 164 (see FIG. 37).

The reliability based on the degree of fat included in the correction region is calculated by calculating a region mean signal ratio ln(R2/G2) and a region mean signal ratio ln(B1/G2) using the mean value of the G2 image signal and the mean value of the R2 image signal in each correction region of the white-light-equivalent image (i.e., the means of the pixel values, in each correction region, of the G channel and the R channel of the white-light-equivalent image) and the mean value of the B1 image signal in each correction region of the first blue light image and applying these signal ratios to a third reliability calculation table 765 as illustrated in FIG. 103 obtained by setting the signal ratio ln(B1/G2) on the vertical axis of the third reliability calculation table 165 (see FIG. 38). The region reliability calculated by the region reliability calculation unit 630 is transmitted to the region reliability determination unit 640 of the correction determination unit 170 (see FIG. 101).

A method of determining the degree of influence of disturbances and performing notification according to the fourth embodiment will be described hereinafter. The region reliability determination unit 640 outputs a determination result as to whether each correction region in the white-light-equivalent image, the first blue light image, and the third illumination light image is a high-reliability correction region or a low-reliability correction region, using a region reliability determination threshold value set in advance.

The region reliability determination threshold value may be set in accordance with the type of region reliability. For example, a first region reliability determination threshold value is set for the “mean value of the pixel values in the correction region”, and if the “mean value of the pixel values in the correction region” is greater than or equal to the first region reliability determination threshold value, the correction region is determined to be a “high-reliability correction region”. On the other hand, if the “mean value of the pixel values in the correction region” is less than the first region reliability determination threshold value, the correction region is determined to be a “low-reliability correction region”.

Likewise, a second region reliability determination threshold value is set for the “standard deviation of the pixel values in the correction region”, a third region reliability determination threshold value is set for the “effective pixel ratio in the correction region”, a fourth region reliability determination threshold value is set for the “reliability regarding the brightness in the correction region”, a fifth region reliability determination threshold value is set for the “reliability based on the degree of fat included in the correction region”, and the determination results are output.

Further, the region reliability determination unit 640 determines the reliability for each of the white-light-equivalent image, the first blue light image, and the third illumination light image using the determination result indicating whether each correction region is a high-reliability correction region or a low-reliability correction region. In this case, an image determination result is output in accordance with the number of correction regions determined to be “low-reliability correction regions” among all the correction regions in the white-light-equivalent image, the first blue light image, and the third illumination light image. The image determination result is output by, for example, setting a first image determination result output threshold value in advance.

For example, the first image determination result output threshold value is set to “10”, and the number of correction regions in the white-light-equivalent image is 16. In this case, if the number of low-reliability correction regions in the white-light-equivalent image is greater than or equal to 10, an image determination result indicating that the reliability of the entire correction regions is high, that is, the influence of disturbances is small and the correction processing can be appropriately performed, is output. On the other hand, if the number of low-reliability correction regions in the white-light-equivalent image is less than 10, an image determination result indicating that the reliability of the entire correction regions is low, that is, the correction processing cannot be appropriately performed due to the influence of some disturbance, is output.

The calculation of the region reliability and the output of the image determination result may be performed on all of the white-light-equivalent image, the first blue light image, and the third illumination light image, or may be performed on some of the white-light-equivalent image, the first blue light image, and the third illumination light image. The reason is to speed up the calculation process.

The image determination result output from the region reliability determination unit 640 is transmitted to the extension display control unit 200. The extension display control unit 200 may change the display mode on the display in accordance with the image determination result. For example, when an image determination result indicating that “the reliability of the entire correction regions is high” is output, the extension display control unit 200 causes the display to display a message indicating that the correction processing can be appropriately performed (see FIG. 41). On the other hand, when an image determination result indicating that “the reliability of the entire correction regions is low” is output, a message such as “Please operate the endoscope for correction processing” is displayed on the display as a warning display (see FIG. 42). These messages may be superimposed on the white-light-equivalent image displayed on the display.

The region reliability determination unit 640 may calculate the image determination average reliability using each correction region in the white-light-equivalent image, the first blue light image, and the third illumination light image. The image determination average reliability is calculated by, for example, dividing the sum of the reliabilities of all the correction regions in the white-light-equivalent image by the number of correction regions. In this case, the region reliability determination unit 640 sets a second image determination result output threshold value in advance for the image determination average reliability, and if the image determination average reliability is greater than or equal to the second image determination result output threshold value, the region reliability determination unit 640 outputs an image determination result indicating that “the reliability of the entire correction regions is high”. On the other hand, if the image determination average reliability is less than the second image determination result output threshold value, the region reliability determination unit 640 outputs an image determination result indicating that “the reliability of the entire correction regions is low”. Also in this case, the extension display control unit 200 may change the display mode on the display in accordance with the image determination result.

A method of determining the degree of movement of the endoscope 12 and performing notification according to the fourth embodiment will be described hereinafter. In this case, the determination result as to whether each correction region in the white-light-equivalent image, the first blue light image, and the third illumination light image is a high-reliability correction region or a low-reliability correction region, which is output from the region reliability determination unit 640, is transmitted to the second pigment value calculation unit 650 (see FIG. 101).

The second pigment value calculation unit 650 may perform an exclusion process for excluding a correction region determined to be a “low-reliability correction region” among the correction regions in the white-light-equivalent image, the first blue light image, and the third illumination light image from the calculation of the second pigment value.

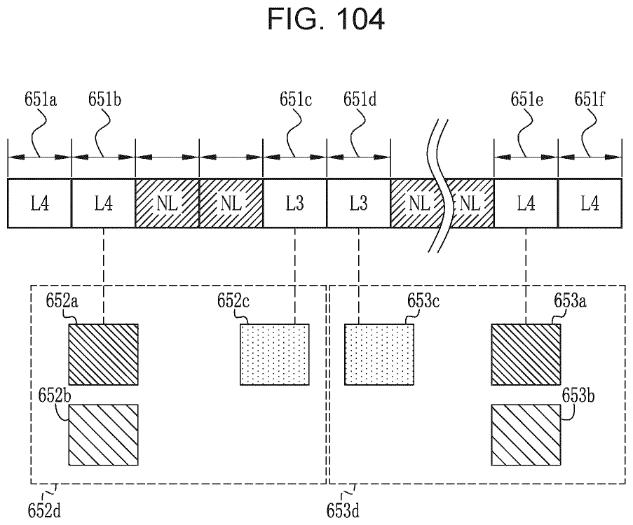

The second pigment value calculation unit 650 may perform the exclusion process on images that are some of the white-light-equivalent image, the first blue light image, and the third illumination light image. Specifically, such images are, for example, as illustrated in FIG. 104, a white-light-equivalent image 652a and a first blue light image 652b generated based on image signals output in, among frames 651a, 651b, 651c, 651d, 651e, and 651f in which the fourth illumination light L4 or the third illumination light L3 is emitted in the light emission pattern in the correction mode according to the fourth embodiment (see FIG. 96), the frame 651b, a third illumination light image 652c generated based on image signals output in the frame 651c, a third illumination light image 653c generated based on image signals output in the frame 651d, and a white-light-equivalent image 653a and a first blue light image 653b generated based on image signals output in the frame 651e.

The white-light-equivalent image 652a, the first blue light image 652b, and the third illumination light image 652c are referred to as a first image set 652d. The white-light-equivalent image 653a, the first blue light image 653b, and the third illumination light image 653c are referred to as a second image set 653d. The second pigment value calculation unit 650 may perform the exclusion process on the images included in the first image set 652d and the images included in the second image set 653d. A correction region determined as a “high-reliability correction region” on which the exclusion process is not to be performed is hereinafter referred to as an effective region. In contrast, a correction region determined as a “low-reliability correction region” on which the exclusion process is to be performed is referred to as an exclusion region.

The second pigment value calculation unit 650 performs the exclusion process such that the positions of the effective regions and the positions of the exclusion regions included in the first image set 652d correspond across the white-light-equivalent image 652a, the first blue light image 652b, and the third illumination light image 652c.

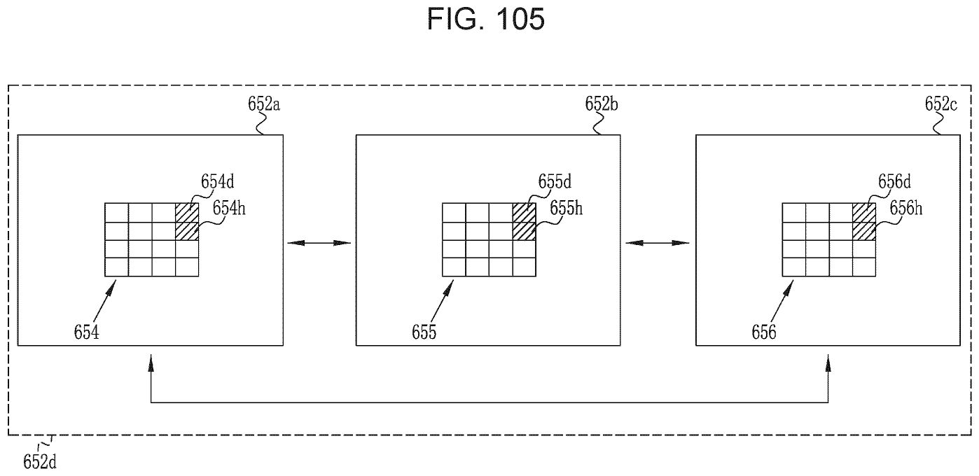

Specifically, for example, as illustrated in FIG. 105, the exclusion regions in the white-light-equivalent image 652a are set to correction regions 654d and 654h, and the correction regions other than the correction regions 654d and 654h in an entire correction region 654 are set as effective regions. Likewise, the exclusion regions in the first blue light image 652b are set to correction regions 655d and 655h, and the correction regions other than the correction regions 655d and 655h in an entire correction region 655 are set as effective regions. The exclusion regions in the third illumination light image 652c are set to correction regions 656d and 656h, and the correction regions other than the correction regions 656d and 656h in an entire correction region 656 are set as effective regions. Through the exclusion process described above, the positions of the effective regions can be made to correspond across the images included in the first image set 652d.

A method of the exclusion process will be described. The second pigment value calculation unit 650 performs the exclusion process on each image set by using an exclusion process threshold value set in advance. The exclusion process threshold value is set as a plurality of values such that the region reliability of each correction region can be evaluated and calculated at five levels from “1” to “5”. The exclusion process threshold value may be set in accordance with the type of region reliability.

First, the second pigment value calculation unit 650 calculates the five levels of region determination reliability for the correction regions in the white-light-equivalent image, the first blue light image, and the third illumination light image included in the image set. Next, the second pigment value calculation unit 650 selects the correction region having the minimum level of region determination reliability from among the corresponding correction regions in the white-light-equivalent image, the first blue light image, and the third illumination light image included in the image set.

Next, the second pigment value calculation unit 650 applies a region reliability determination threshold value for determining a “high-reliability correction region” or a “low-reliability correction region” to the correction region having the minimum level of region determination reliability, and sets a correction region determined to be a “low-reliability correction region” as an exclusion region. In this case, all the correction regions in the white-light-equivalent image, the first blue light image, and the third illumination light image corresponding to the correction region determined to be a “low-reliability correction region” are set as exclusion regions.

The second pigment value calculation unit 650 calculates a second pigment value from each of the first image set 652d and the second image set 653d. The calculation of the second pigment value will be specifically described hereinafter. When the second pigment value of the first image set 652d is to be calculated, the region mean signal ratio ln(R2/G2) as the X-component value, the region mean signal ratio ln(B1/G2) as the Y-component value, and the region mean signal ratio ln(B3/G3) as the Z-component value are calculated for each effective region, based on the signal values in the effective regions at corresponding positions in the white-light-equivalent image 652a, the first blue light image 652b, and the third illumination light image 652c.

The region mean signal ratio ln(R2/G2) is calculated using the mean value of the R2 image signal in each effective region of the white-light-equivalent image 652a and the mean value of the G2 image signal in each effective region of the white-light-equivalent image 652a (i.e., the mean of the pixel values in each effective region of the R channel and the mean of the pixel values in each effective region of the G channel of the white-light-equivalent image).

The region mean signal ratio ln(B1/G2) is calculated using the mean value of the B1 image signal in each effective region of the first blue light image 652b (i.e., the mean of the pixel values in each effective region of the first blue light image) and the mean value of the G2 image signal in each effective region of the white-light-equivalent image 652a.

The region mean signal ratio ln(B3/G3) is calculated using the mean value of the B3 image signal in each effective region of the third illumination light image 652c and the mean value of the G3 image signal in each effective region of the third illumination light image 652c (i.e., the mean of the pixel values in each effective region of the B channel and the mean of the pixel values in each effective region of the G channel of the third illumination light image).

The second pigment value calculation unit 650 calculates the region mean signal ratio ln(R2/G2), the region mean signal ratio ln(B1/G2), and the region mean signal ratio ln(B3/G3) calculated for the respective corresponding effective regions of the first image set 652d by referring to the corrected oxygen saturation calculation table 120 (see FIG. 29A). In the corrected oxygen saturation calculation table 120, the curved surfaces CV0 to CV4 are distributed in the three-dimensional coordinate system in which the signal ratio ln(R2/G2) is on the X-axis, the signal ratio ln(B1/G2) is on the Y-axis, and the signal ratio ln(B3/G3) is on the Z-axis, in accordance with the concentration of the yellow pigment.

The second pigment value calculation unit 650 refers to the corrected oxygen saturation calculation table 120, which is a three-dimensional coordinate system, and calculates the second pigment value using a curved surface on which the coordinates (X4, Y4, Z4)=(region mean signal ratio ln(R2/G2), region mean signal ratio ln(B1/G2), region mean signal ratio ln(B3/G3)) overlap or are closest among the curved surfaces CV0 to CV4. It is indicated that the second pigment values of the curved surfaces CV0 to CV4 are “0” to “4”, respectively. For example, when the coordinates (X4, Y4, Z4) overlap on the curved surface CV2, the second pigment value is calculated as “2”. The second pigment value calculation unit 650 calculates the second pigment value for each effective region of the first image set 652d. The second pigment value of the second image set 653d is also calculated for each effective region in a similar manner.

The second pigment value calculated for each effective region of the first image set 652d and the second pigment value calculated for each effective region of the second image set 653d are transmitted to the second pigment value determination unit 660 of the correction determination unit 170. It is preferable that the X-component value, the Y-component value, and the Z-component value calculated for each effective region of the first image set 652d and the X-component value, the Y-component value, and the Z-component value calculated for each effective region of the second image set 653d are transmitted to the second pigment value determination unit 660.

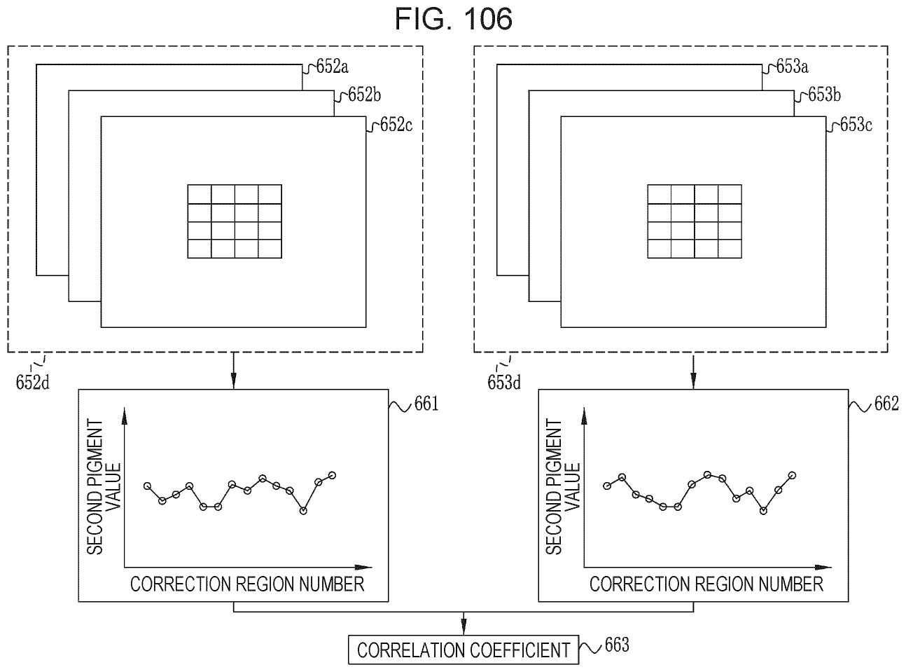

As described with reference to FIG. 106, the second pigment value determination unit 660 determines a correlation coefficient 663 between a second pigment value 661 calculated for each effective region of the first image set 652d and a second pigment value 662 calculated for each effective region of the second image set 653d. In FIG. 106, for description, regarding the second pigment value 661 calculated for each effective region and the second pigment value 662 calculated for each effective region, the vertical axis represents the second pigment values, and the horizontal axis represents the numbers of the correction regions assigned to the effective regions (i.e., the number “N” of the N-th correction region).

If the correlation coefficient is smaller than a movement determination threshold value set in advance, the second pigment value determination unit 660 determines that “the degree of movement of the endoscope is large”. On the other hand, if the correlation coefficient is larger than the movement determination threshold value, the second pigment value determination unit 660 determines that “the degree of movement of the endoscope is small”. In this case, the second pigment value determination unit 660 outputs, as a movement determination result, the determination result indicating that “the degree of movement of the endoscope is large” or “the degree of movement of the endoscope is small”, and transmits the movement determination result to the extension display control unit 200.

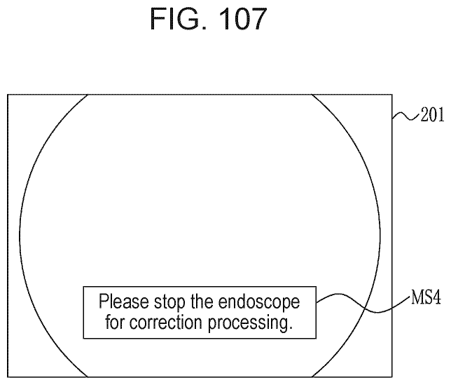

The extension display control unit 200 may change the display mode on the display in accordance with the movement determination result. For example, if the movement determination result indicating that “the degree of movement of the endoscope is small” is output, the extension display control unit 200 causes the display to display a message indicating that the correction processing can be appropriately performed (see FIG. 41). On the other hand, if the movement determination result indicating that “the degree of movement of the endoscope is large” is output, a message MS4 such as “Please stop the endoscope for correction processing” as illustrated in FIG. 107 is displayed on the display as a warning display. These messages may be superimposed on the white-light-equivalent image 201.

In a case where a light emission switching instruction is input by manual input to switch the illumination light, the endoscope 12 may largely move during the switching. In this case, the correction processing may be difficult to appropriately perform in accordance with the influence of the concentration of the specific pigment. Accordingly, the degree of movement of the endoscope 12 is determined, and the user is notified when the degree of movement is large, thus making it possible to prompt the user not to move the endoscope 12. As a result, when the degree of movement of the endoscope 12 is small, the correction processing can be appropriately performed.

As described above, the image determination result obtained by determining the degree of influence of disturbances and/or the movement determination result obtained by determining the degree of movement of the endoscope 12 is notified to the user. Thus, the user can be prompted to perform an operation for appropriately performing the correction processing. In the correction processing, to perform the table correction processing according to the first pigment value, it is preferable to determine first pigment values by a robust estimation method based on the second pigment values for each correction region calculated using the first image set and the second image set and to select an oxygen saturation calculation table that is the areas AR0 to AR4 corresponding to the first pigment values. When the movement determination result indicating that “the degree of movement of the endoscope is small” is output and the image determination result indicating that “the reliability of the entire correction regions is high” is output, a first pigment value may be determined using the region mean signal ratio in a correction region determined as a “high-reliability correction region” to perform the correction processing.

In the embodiments described above, the hardware structures of processing units that perform various processes, such as the image signal acquisition unit 60, the endoscopic image generation unit 70, the display control unit 80, the image communication unit 90, the oxygen saturation image generation unit 130, the corrected oxygen saturation calculation unit 140, the table correction unit 141, the extension central control unit 150, the reliability calculation unit 160, the correction determination unit 170, the extension display control unit 200, the region-of-interest setting unit 210, the region index value calculation unit 250, the index value display table generation unit 260, the display image generation unit 270, and the index value link line generation unit 290, are various processors as follows. The various processors include a central processing unit (CPU) as a general-purpose processor that executes software (program) to function as various processing units, a programmable logic device (PLD) as a processor whose circuit configuration can be changed after manufacturing, such as a field programmable gate array (FPGA), a dedicated electric circuit as a processor having a circuit configuration designed exclusively for executing various processes, and so on.

A single processing unit may be configured as one of the various processors or as a combination of two or more processors of the same type or different types (for example, a plurality of FPGAs or a combination of a CPU and an FPGA). Alternatively, a plurality of processing units may be configured as a single processor. Examples of configuring a plurality of processing units as a single processor include, first, a form in which, as typified by a computer such as a client or a server, the single processor is configured as a combination of one or more CPUs and software and the processor functions as the plurality of processing units. The examples include, second, a form in which, as typified by a system on chip (SoC) or the like, a processor is used in which the functions of the entire system including the plurality of processing units are implemented as one integrated circuit (IC) chip. As described above, the various processing units are configured by using one or more of the various processors described above as a hardware structure.

More specifically, the hardware structure of these various processors is an electric circuit (circuitry) in which circuit elements such as semiconductor elements are combined. The hardware structure of a storage unit is a storage device such as a hard disc drive (HDD) or a solid state drive (SSD).

### REFERENCE SIGNS LIST

10 endoscope system
12 endoscope
12a insertion section
12b operation section
12c mode switching switch
12d region-of-interest setting switch
13 light source device
14 processor device
15 first user interface
16 extension processor device
17 second user interface
20 light source unit
20a V-LED
20b BS-LED
20c BL-LED
20d G-LED
20e R-LED
21 light source control unit
41 light guide
42 illumination optical system
42a illumination lens
43 imaging optical system
43a objective lens
44, 511, 512, 513, 514, 611, 612 imaging sensor
45 imaging control unit
46 CDS/AGC circuit
47 A/D converter
50 central control unit
60 image signal acquisition unit
70 endoscopic image generation unit
80 display control unit
81 white-light image
82 notification image
90 image communication unit
100, 103 hemoglobin reflection spectrum
101a, 101b, 101c, 102a, 102b curve
104 absorption spectrum of yellow pigment
110 oxygen saturation calculation table
120 corrected oxygen saturation calculation table
121 three-dimensional coordinate system
122, 430 two-dimensional coordinate system
130 oxygen saturation image generation unit
131 base image generation unit
132 arithmetic value calculation unit
133 oxygen saturation calculation unit
134 color tone adjustment unit
140 corrected oxygen saturation calculation unit
141 table correction unit
150 extension central control unit
160 reliability calculation unit
161 correction image
162 specific region
163, 763 first reliability calculation table
164 second reliability calculation table
165, 765 third reliability calculation table
170 correction determination unit
171a low-reliability region
171b high-reliability region
172 frame
200 extension display control unit
201, 213, 621, 652a, 653a white-light-equivalent image
202 oxygen saturation image
210 region-of-interest setting unit
211, 214, 215 region-of-interest image
212a, 212b, 212c region of interest
212d display region of interest
220a, 220b, 220c lock-on area
220d display lock-on area
240 region position information storage unit
250 region index value calculation unit
251a, 251b, 251c, 282a, 282b region index value
252a first time point region index value
252b second time point region index value
252c third time point region index value
260 index value display table generation unit
261, 263, 264, 265 index value display table
262, 266, 267, 268 line sparkline
269, 269a, 269b extension index value display table
270 display image generation unit
271, 273, 275 display image
272a, 272b, 272c, 274a, 274b, 274c, 276a, 276b, 276c region position information
272d, 278c display-region position information
277, 278a, 278b additional region of interest
280 region index value storage unit
281 out-of-field-of-view lock-on area index value
283, 285a, 285b additional region index value
290 index value link line generation unit index value link line
291a, 291b, 291c, 292a, 292b, 292c
350 combination index calculation table
351 biological index value selection screen
352 radio button
400 broadband light source
410 rotary filter
411 inner filter
411a, 412a B1 filter
411b, 412c G FILTER
411c, 412d R filter
412 outer filter
412b B2 filter
412e B3 filter
420 filter switching unit
431a reference line
431b actual measurement line
500, 600 camera head
501, 502, 503, 601 dichroic mirror
620 feature value calculation unit
622, 654, 654d, 654h, 655, 655d, 655h, 656, 656d, 656h correction region
622a first correction region
622p sixteenth correction region
630 region reliability calculation unit
640 region reliability determination unit
650 second pigment value calculation unit
651a, 651b, 651c, 651d, 651e, 651f frame
652b, 653b first blue light image
652c, 653c third illumination light image
652d first image set
653d second image set
660 second pigment value determination unit
661, 662 second pigment value
663 correlation coefficient
Ot operating table
P subject
AC abdominal cavity
Tr trocar
To treatment tool
V violet light
BS second blue light
BL first blue light
DFX, DFY definition line
G green light
R red light
Lc white light
L1 first illumination light
L2 second illumination light
L3 third illumination light
Pc white-light illumination period
P1 first illumination period
P2 second illumination period
P3 third illumination period
BF B color filter
GF G color filter
RF R color filter
EL, ELH, ELL contour line
MS0, MS1, MS2, MS3, MS4 message
CV0, CV1, CV2, CV3, CV4 curved surface
AR0, AR1, AR2, AR3, AR4 area

What is claimed is:

1. An endoscope system comprising:
an endoscope that captures an image of a photographic subject to generate an image signal; and
a processor,
the processor being configured to:
acquire the image signal;
generate an endoscopic image based on the image signal;
set a plurality of regions of interest at different positions in the endoscopic image;
store each of the positions of the plurality of regions of interest in the endoscopic image as region position information;
calculate a biological index value indicating a state of the photographic subject, based on the image signal in the regions of interest;
calculate, for each of the regions of interest, a region index value that is a statistical value of the biological index value, based on the biological index value in each of the regions of interest;
generate an index value display table that collectively displays a plurality of the region index values;
generate a display image that displays the endoscopic image, the index value display table, and a plurality of pieces of the region position information; and
perform control to display the display image.

2. The endoscope system according to claim 1, wherein the biological index value is an oxygen saturation and/or a hemoglobin index.

3. The endoscope system according to claim 2, wherein the index value display table displays the plurality of the region index values in a graph format.

4. The endoscope system according to claim 3, wherein the processor is configured to:
associate the region position information and the region index value with each other to store the region index value as a specific region index value; and
hold the specific region index value and display the specific region index value in the index value display table.

5. The endoscope system according to claim 3, wherein the processor is configured to:
calculate the biological index value based on the image signal that is latest in the regions of interest;
calculate, for each of the regions of interest, the region index value based on the biological index value that is latest; and
update the region index value displayed in the index value display table.

6. The endoscope system according to claim 4, wherein the processor is configured to:
associate the region position information and each of the regions of interest with each other to store the position of the region of interest in the endoscopic image as a lock-on area; and
calculate the biological index value based on the image signal in the lock-on area.

7. The endoscope system according to claim 6, wherein the processor is configured to:
associate the region index value calculated based on the image signal in the lock-on area with the lock-on area to store the region index value as a specific lock-on area index value; and
perform control to display the specific lock-on area index value on the display image.

8. The endoscope system according to claim 7, wherein when the lock-on area is at a position out of a field of view, the position out of the field of view being a position not included in the endoscopic image,
the processor is configured to
set, as an out-of-field-of-view lock-on area index value, the specific lock-on area index value stored immediately before the lock-on area is located at the position out of the field of view, and generate the index value display table that displays the out-of-field-of-view lock-on area index value.

9. The endoscope system according to claim 8, wherein the processor is configured to:
set at least one lock-on area in the endoscopic image as an additional region of interest, each of the at least one lock-on area being the lock-on area;
calculate the biological index value based on the image signal in the additional region of interest;
calculate an additional region index value as the region index value, the additional region index value being a statistical value of the biological index value in the additional region of interest;
generate an extension index value display table that collectively displays the additional region index value and the out-of-field-of-view lock-on area index value; and
perform control to display the extension index value display table on the display image.

10. The endoscope system according to claim 9, wherein the processor is configured to:
perform control to display a plurality of pieces of the region position information in a superimposed manner on the endoscopic image; and
perform control to display an index value link line on the display image, the index value link line connecting the region position information displayed in a superimposed manner on the endoscopic image and the additional region index value other than the out-of-field-of-view lock-on area index value, the additional region index value other than the out-of-field-of-view lock-on area index value being displayed in the extension index value display table and corresponding to the region position information.

11. The endoscope system according to claim 10, wherein the processor is configured to
perform control to change a display size of the extension index value display table displayed on the display image.

12. The endoscope system according to claim 7, wherein the processor is configured to:
perform control to display a plurality of pieces of the region position information in a superimposed manner on the endoscopic image; and
perform control to display an index value link line on the display image, the index value link line connecting the region position information displayed in a superimposed manner on the endoscopic image and the region index value displayed in the index value display table and corresponding to the region position information.

13. The endoscope system according to claim 7, wherein the processor is configured to:
set at least one lock-on area in the endoscopic image as an additional region of interest, each of the at least one lock-on area being the lock-on area;
calculate the biological index value based on the image signal in the additional region of interest;
calculate an additional region index value as the region index value, the additional region index value being a statistical value of the biological index value in the additional region of interest;
generate the index value display table that displays the additional region index value; and
perform control to display, on the display image, the index value display table that displays the additional region index value.

14. The endoscope system according to claim 1, further comprising a region-of-interest setting switch, wherein
the processor is configured to:
set the plurality of regions of interest in accordance with a pressing of the region-of-interest setting switch; and
calculate the region index value in the set regions of interest in accordance with a further pressing of the region-of-interest setting switch.

15. A method for operating an endoscope system, comprising the steps of:
acquiring an image signal generated by an endoscope capturing an image of a photographic subject;
generating an endoscopic image based on the image signal;
setting a plurality of regions of interest at different positions in the endoscopic image;
storing each of the positions of the plurality of regions of interest in the endoscopic image as region position information;
calculating a biological index value indicating a state of the photographic subject, based on the image signal in the regions of interest;
calculating, for each of the regions of interest, a region index value that is a statistical value of the biological index value, based on the biological index value in each of the regions of interest;
generating an index value display table that collectively displays a plurality of the region index values;
generating a display image that displays the endoscopic image, the index value display table, and a plurality of pieces of the region position information; and
performing control to display the display image.
